(19) United States
(12) Patent Application Publication
(10) Pub. No.: US 2025/0229084 A1
(43) Pub. Date: Jul. 17, 2025

(54) Tumor Electric Field Treatment System

(71) Applicant: JIANGSU HEALTHY LIFE INNOVATION MEDICAL TECHNOLOGY CO., LTD, Wuxi, Jiangsu (CN)

(72) Inventors: Qichao SHEN, Jiangsu (CN); Jianjun YING, Jiangsu (CN); Jing YU, Jiangsu (CN); Jun ZHANG, Jiangsu (CN); Sheng CHEN, Jiangsu (CN); Hu SUN, Jiangsu (CN); Jiajie HUI, Jiangsu (CN); Yidong SUN, Jiangsu (CN)

(21) Appl. No.: 18/696,568

(22) PCT Filed: Nov. 28, 2022
(86) PCT No.: PCT/CN2022/134677

§ 371 Date: Mar. 17, 2025

(30) Foreign Application Priority Data
Sep. 28, 2021 (CN) 202111143956.3
Dec. 22, 2021 (CN) 202111578521.1
Dec. 22, 2021 (CN) 202111578531.5
Dec. 22, 2021 (CN) 202111578597.4
Dec. 22, 2021 (CN) 202111580039.1
Dec. 22, 2021 (CN) 202111580040.4
Dec. 22, 2021 (CN) 202111580105.5
Dec. 22, 2021 (CN) 202111580121.4
Dec. 22, 2021 (CN) 202111580130.3
Dec. 22, 2021 (CN) 202111580142.6
Dec. 22, 2021 (CN) 202111580196.2
Dec. 22, 2021 (CN) 202123242599.4
Dec. 31, 2021 (CN) 202111667243.7

(51) Int. Cl. A61N 1/36; A61N 1/02; A61N 1/04

(52) U.S. Cl. CPC A61N 1/36034; A61N 1/025; A61N 1/0476; A61N 1/36002

(57) ABSTRACT

The present application provides a tumor electric field treatment system. The tumor electric field treatment system comprises an electric field treatment device and two pairs of the insulated electrodes electrically connected to the electric field treatment device. The AC signal controller is configured to generate control signals with respective time periods and at least two cyclically switched output states. The AC signal generator is configured to generates an alternating electric signal, and according to the output state of the control signals generated by the AC signal controller, cyclically and alternately apply the generated alternating electric signal to the insulated electrodes in pairs, so as to alternately generate alternating electric fields between the two pairs of insulated electrodes. The alternating electric signal applied to the insulated electrodes in pair have respective continuous on-time periods, and each continuous-on time period comprises an initial switching-on time period, an intermediate on-time period and a final switching-off time period. During the switching-on time period, the AC voltage value of the alternating electric signal applied to the insulated electrodes in pair rises from 0 to a specific voltage at a constant speed. During the switching-off time period, the AC voltage value of the alternating electric signal applied to the insulated electrodes in pair decreases from a specific voltage to 0 at a constant speed.

This application claims the benefit of priorities to the following patent applications: Chinese Patent Application No. CN202111578597.4 with an application date of Dec. 22, 2021; Chinese Patent Application No. CN202111667243.7 filed on Dec. 31, 2021; Chinese Patent Application No. CN202111580105.5 filed on Dec. 22, 2021; Chinese Patent Application No.
CN202111580121.4 filed on Dec. 22, 2021; Chinese Patent Application No.
CN202111580130.3 filed on Dec. 22, 2021; Chinese Patent Application No.
CN202111580142.6 filed on Dec. 22, 2021; Chinese Patent Application No.
CN202111578521.1 filed on Dec. 22, 2021; Chinese Patent Application No.
CN202111580040.4 filed on Dec. 22, 2021; Chinese Patent Application No.
CN202111580039.1 filed on Dec. 22, 2021; Chinese Patent Application No.
CN202123242599.4 filed on Dec. 22, 2021; Chinese Patent Application No.
CN202111578531.5 filed on Dec. 22, 2021; Chinese Patent Application No.
CN202111580196.2 filed on Dec. 22, 2021, and Chinese Patent Application No.
CN202111143956.3 with an application date of Sep. 28, 2021, which are incorporated herein by reference in their entireties.

### TECHNICAL FIELD

The present disclosure relates to a tumor electric field therapy system, pertaining to the field of medical technology and equipment.

### BACKGROUND

A tumor electric field therapy system and an electric filed application method thereof are disclosed in a Chinese Invention Patent issued as CN104771830B. The tumor electric field therapy system includes an electric field treatment device generating an alternating electrical signal with alternating voltage and two pairs of insulated electrodes electrically connected to the electric field treatment device. Two pairs of the insulated electrodes arranged perpendicularly to each other and around malignant tumor sites in experimental animals or around proliferating cells in tissue culture periodically and alternately apply the alternating electrical signal generated by the electric field treatment device to the malignant tumor sites in the experimental animals or the proliferating cells in the tissue culture to treat the malignant tumors in the experimental animals or inhibit the proliferating cells in the tissue culture. The electric field treatment device includes an Alternating Current (AC) signal generator and an AC signal controller electrically connected to the AC signal generator. The AC signal controller generates a periodic control signal with two output states to control the AC signal generator to generate an alternating signals alternately applied between two pairs of insulated electrodes. Specifically, when the AC signal controller is in a first output state, the AC signal controller controls the AC signal generator to generate an alternating signal between a first pair of the insulated electrodes, but no alternating signal is generated between a second pair of the insulated electrodes; and when the AC signal controller is in a second output state, the AC signal controller controls the AC signal generator to generate an alternating signal between the second pair of the insulated electrodes, but no alternating signal is generated between the first pair of the insulated electrodes. That is, when the alternating electric signal between the first pair of the insulated electrodes is conducted, the alternating electric signal between the second pair of the insulated electrodes is interrupted; and when the alternating electric signal between the first pair of the insulated electrodes is interrupted, the alternating electric signal between the second pair of the insulated electrodes is conducted.

The aforementioned tumor electric field therapy system achieves a switching of the alternating electric signal generated by the AC signal generator and applied between two pairs of the insulated electrodes by controlling the switching of the AC signal controller between the first and second output states, thereby alternately applying the alternating electric fields in different directions to the malignant tumor sites of experimental animals or proliferating cells in tissue cultures to treat tumors or inhibit the proliferating cells. Although the tumor electric field therapy system uses the AC signal controller to alternately apply the alternating electric fields to the malignant tumor sites of the experimental animals or the proliferating cells in the tissue culture through two pairs of the insulated electrodes to achieve the purpose of treating tumors or inhibiting the proliferating cells, the alternating electric signals between two pairs of the insulated electrodes have voltage mutations when switched from zero to a certain value or from the certain value to zero. When the alternating electric signal has a large voltage variation within per unit time, the AC signal controller generates signal fluctuations and signal spikes to impact and damage electronic components located therein. Furthermore, the voltage mutations of the alternating electric signals may also cause scalp tingling and uncomfortable in a patient during using the tumor electric field therapy system.

Therefore, it is certainly necessary to provide an improved tumor electric field therapy system to inhibit the proliferation of tumor cells.

### SUMMARY

The present disclosure provides an improved tumor electric field therapy system to avoid impacting or damaging electronic elements due to spike signals generated when an alternating electric signal is switched.

The tumor electric filed therapy system can be implemented through the following technical solutions: A tumor electric field therapy system includes an electric field treatment device and at least two pairs of insulated electrodes electrically connected to the electric field treatment device. The electric field treatment device includes an AC signal generator and an AC signal controller electrically connected to the AC signal generator. The AC signal controller is configured to generate control signals with respective time periods and at least two cyclically switched output states. The AC signal generator is configured to generate an alternating electric signal with an AC voltage and a 150 kHz frequency for treating tumor and is also configured to cyclically and alternately apply the generated alternating electric signal to the insulated electrodes arranged in pairs for generating alternating electric fields with cyclically switched directions when the AC signal controller is cyclically switched between the at least two output states. The AC signal generator is further configured to make alternating electric signals each having respective continuous on-time periods being applied to different pairs of the insulated electrodes. Each continuous on-time period comprises an initial switching-on time period t3 with a preset value, an intermediate on-time period and a final switching-off time period t4 with a preset value. The alternating electric signal has a specific voltage during the intermediate on-time period when applied to the insulated electrodes arranged in pairs. During the switching-on time period t3, the AC voltage of the alternating electric signal applied to the insulated electrodes arranged in pairs rises from 0 to the specific voltage at a constant speed and an AC voltage change of the alternating electric signal per millisecond is within 5% of the specific voltage; and during the switching-off time period t4, the AC voltage of the alternating electric signal applied to the insulated electrodes arranged in pairs decreases from the specific voltage to 0 at a constant speed and an AC voltage change of the alternating electric signal per millisecond is also within 5% of the specific voltage.

According to another aspect of the embodiment of the present disclosure, the electric field treatment device has preset system parameters that include an electric field frequency of the alternating electric signal and an output AC voltage amplitude of the alternating electric signal, and the specific voltage is less than or equal to a peak voltage of the alternating electrical signal output by the electric field treatment device, and the alternating electric signal generated by the AC signal generator is periodically applied to the insulated electrodes arranged in pairs under the control of the AC signal controller.

According to another aspect of the embodiment of the present disclosure, each control signal generated by the AC signal controller has a cyclic switching period with one second between the at least two output states, and each continuous on-time period of the alternating electric signal is one second.

According to another aspect of the embodiment of the present disclosure, during the switching-on time period t3, the AC voltage change ΔV of the alternating electric signal per millisecond is obtained through the following formula: ΔV=V/(t3/t), wherein V is the specific voltage, t is 1 millisecond, and t3 is the switching-on time period of the alternating electric signal and is greater than or equal to 20 milliseconds.

According to another aspect of the embodiment of the present disclosure, during the switching-off time period t4, the AC voltage change ΔV of the alternating electric signal per millisecond is obtained as ΔV=V/(t4/t), wherein V is the specific voltage, t is 1 millisecond, and t4 is the switching-off time period of the alternating electric signal and is greater than or equal to 20 milliseconds.

According to another aspect of the embodiment of the present disclosure, the switching-on time period t3 of the alternating electric signal is equal to the switching-off time period t4 of the alternating electric signal.

According to another aspect of the embodiment of the present disclosure, the switching-on time period t3 of the alternating electric signal is 50 milliseconds.

According to another aspect of the embodiment of the present disclosure, the switching-off time period t4 of the alternating electric signal is 50 milliseconds.

According to another aspect of the embodiment of the present disclosure, the electric field treatment device includes a Micro-Controller unit (MCU) control unit with a reference voltage, a DC power supply control unit communicated with the MCU control unit, an inverter boost control unit communicated with the MCU control unit, a filter control unit connected to the inverter boost control unit, an AC voltage control unit communicated with the filter control unit, a direction control unit communicated with the MCU control unit, a first direction switch electrically connected to the direction control unit and controlling connection and disconnection between the AC voltage control unit and one pair of the insulated electrodes, and a second direction switch electrically connected to the direction control unit and controlling connection and disconnection between the AC voltage control unit and another pair of the insulated electrodes.

According to another aspect of the embodiment of the present disclosure, the MCU control unit includes a storage module storing the system parameters of the electric field treatment device, an execution module communicating with the storage module, a digital-to-analog conversion module communicating with the execution module, and a control module controlling the storage module, the execution module and the digital-to-analog conversion module to perform corresponding operations.

According to another aspect of the embodiment of the present disclosure, the digital-to-analog conversion (DAC) module communicated with the DC power supply control unit has a DAC data register and is configured to output a corresponding DC signal to the DC power supply control unit based on a value stored in the DAC data register to start the DC power supply control unit, and the DC power supply control unit outputs a DC signal to the inverter boost control unit after being started.

According to another aspect of the embodiment of the present disclosure, the storage module is configured to store the system parameters of the electric field treatment device and the system parameters of the electric field treatment device further include a direction switching period of the alternating electric signal.

According to another aspect of the embodiment of the present disclosure, the AC voltage of the alternating electric signal rising from 0 to the specific voltage at a constant speed during the switching-on time period t3 is achieved by the MCU control unit controlling the DC power supply unit to output a DC signal with a voltage increased at a constant speed to the inverter boost control unit based on the direction switching period of the alternating electric signal which is obtained by the execution module.

According to another aspect of the embodiment of the present disclosure, the AC voltage of the alternating electric signal decreasing from the specific voltage to 0 at a constant speed during the switching-off time period t4 is achieved by the MCU control unit controlling the DC power supply unit to output a DC signal with a voltage decreased at a constant speed to the inverter boost control unit based on the direction switching period of the alternating electric signal which is obtained by the execution module.

According to another aspect of the embodiment of the present disclosure, the MCU control unit is further configured to calculate a voltage output increment or a voltage output decrement of the DAC module per millisecond based on the reference voltage, the AC voltage change of the AC voltage control unit per millisecond, the specific voltage and the DAC data register value corresponding to the specific voltage; and the voltage output increment or voltage output decrement of the DAC module per millisecond is determined as ΔV_{DAC}=(3.3*1000*ΔV*DAC)/(4096*V), wherein ΔV_{DAC} is the voltage output increment or decrement of the DAC module per millisecond and is measured in millivolt; 3.3 is the reference voltage of the MCU and is measured in volts; the value corresponding to the reference voltage of the MCU and stored in the DAC data register is 4096 and equal to 2¹²; ΔV is the AC voltage change of the AC voltage control unit per millisecond and is measured in volts; V is the specific voltage and is measured in volts; and DAC is a DAC data register value corresponding to the specific voltage and stored in the DAC data register.

According to another aspect of the embodiment of the present disclosure, the execution module of the MCU control unit is configured to read the electric field frequency and the output AC voltage amplitude of the alternating electric signal from the storage module and is also configured to, basing on the read electric field frequency and the output AC voltage amplitude of the alternating electric signal and the reference voltage of the MCU control unit, output a pulse signal having a frequency same with the read electric field frequency of the alternating electric signal and a voltage amplitude value equal to the reference voltage of the MCU to the inverter boost control unit.

According to another aspect of the embodiment of the present disclosure, the electric field frequency of the alternating electric signal stored in the electric field treatment device is 150 kHz and the peak of the output AC voltage amplitude of the alternating electric signal is 160V.

According to another aspect of the embodiment of the present disclosure, the pulse signal is a square wave with a frequency of 150 kHz, an AC voltage amplitude of 3.3V and a duty cycle of 50%.

According to another aspect of the embodiment of the present disclosure, the inverter boost control unit includes a boost module communicated with the execution module of the MCU control unit and an inverter module communicatively connected to the boost module, and the DC signal is output from the DC power supply control unit to the boost module.

According to another aspect of the embodiment of the present disclosure, the DC signal output from the DC power supply control unit to the boost module has a voltage of 20V, and the boost module outputs a square wave with a frequency of 150 kHz and an AC voltage amplitude of 80V to the inverter module after superposing and boosting the square wave output by the execution module of the MCU and the DC signal with a voltage of 20V from the DC power supply control unit.

According to another aspect of the embodiment of the present disclosure, the inverter module outputs a square wave with a frequency of 150 kHz and an AC voltage amplitude of 80V to the filter control unit after performing an inverter processing for the received square wave with the frequency of 150 kHz and the AC voltage amplitude of 80V from the boost module.

According to another aspect of the embodiment of the present disclosure, the filter control unit outputs a sine-wave signal with a frequency of 150 kHz and an AC voltage amplitude of 160V to the AC voltage control unit after performing a filtering processing for the received square-wave signal from the inverter module, and under the control of the direction control unit, the AC voltage control unit applies the sine-wave signal to the insulated electrodes arranged in pairs.

According to another aspect of the embodiment of the present disclosure, the execution module is configured to output a periodic direction switching drive signal to the direction control unit based on reading the direction switching period of the alternating electric signal predetermined in the electric field treatment device.

According to another aspect of the embodiment of the present disclosure, the direction control unit controls the connection and disconnection between the first direction switch, the second direction switch and the AC voltage control unit based on the periodic direction switching drive signal output by the control module of the MCU control unit.

According to another aspect of the embodiment of the present disclosure, the AC signal generator is mainly composed of the storage module of the MCU control unit, the execution module of the MCU control unit, the digital-to-analog conversion module of the MCU control unit, the control module of the MCU control unit, the DC power supply control unit, the inverter boost control unit, the filter control unit and the AC voltage control unit.

According to another aspect of the embodiment of the present disclosure, the AC signal controller is mainly constituted by the storage module of the MCU control unit, the execution module of the MCU control unit, the control module of the MCU control unit, the direction control unit, the first direction switch and the second direction switch electrically connected to the direction control unit.

According to another aspect of the embodiment of the present disclosure, the first direction switch is an X-direction switch, the second direction switch is a Y-direction switch, and based on the periodic direction switching drive signal output by the MCU control unit, the direction control unit controls the connection and disconnection of the X-direction switch and the Y-direction switch to periodically and alternately apply the alternating electric signal output by the AC voltage control unit to the corresponding insulated electrodes arranged in pairs.

According to another aspect of the embodiment of the present disclosure, the periodic direction switching drive signal output by the control module of the MCU control unit is generated by the MCU control unit based on the direction switching period of the alternating electric signal of the electric field treatment device.

According to another aspect of the embodiment of the present disclosure, the MCU control unit calculates a numerical output increment of the DAC data register of the digital-to-analog conversion module per millisecond based on a DC voltage change of the DC power supply control unit per millisecond.

According to another aspect of the embodiment of the present disclosure, the MCU control unit calculates a numerical output decrement of the DAC data register of the digital-to-analog conversion module per millisecond based on a DC voltage change of the DC power supply control unit per millisecond.

According to another aspect of the embodiment of the present disclosure, at least two pairs of the insulated electrodes are arranged in pairs and around a malignant tumor site or proliferating cells in tissue culture.

According to another aspect of the embodiment of the present disclosure, AC signal controller has various output states and the number of the output states is the same as the number of pairs of the insulated electrodes arranged in pairs.

According to another aspect of the embodiment of the present disclosure, the AC signal controller has various output states which have respective and non-overlapped time periods corresponding thereto, and the AC signal controller only has one output state in each time period.

According to another aspect of the embodiment of the present disclosure, the AC signal generator is configured to selectively apply the generated alternating electric signal to one pair of the insulated electrodes among at least two pairs of the insulated electrodes based on the output state of the AC signal controller.

According to another aspect of the embodiment of the present disclosure, the at least two pairs of the insulated electrodes comprise a first pair of the insulated electrodes and a second pair of the insulated electrodes all arranged on a surface of a patient's torso; the AC signal controller is configured to generate a periodic control signal having a first output state with a duration between 500 ms and 980 ms and a second output state with a duration between 500 ms and 980 ms; the AC signal generator generates a first AC signal with a frequency of 150 kHz applied to the first pair of the insulated electrodes when the control signal is in the first output state and generates a second AC signal with a frequency of 150 kHz applied to the second pair of the insulated electrodes when the control signal is in the second output state, and a switch between applying the first AC signal to the first pair of the insulated electrodes and applying the second AC signal to the second pair of the insulated electrodes is achieved by switching between the first output state and the second output state of the control signal.

According to another aspect of the embodiment of the present disclosure, the first output state has a duration with a first time period T1, the second output state has a duration with a second time period T2, and the first time period T1 is the same as the second time period T2.

According to another aspect of the embodiment of the present disclosure, the first time period T1 and the second time period T2 are both 50% of an operating period.

According to another aspect of the embodiment of the present disclosure, the first AC signal has an increasing amplitude during the switching-on time period t3 and a decreasing amplitude during the switching-off time period t4 within each of the first time periods T1; and the second AC signal has an increasing amplitude during the switching-on time period t3 and a decreasing amplitude during the switching-off time period t4 within each of the second time periods T2.

According to another aspect of the embodiment of the present disclosure, the switching-on time period t3 and the switching-off time period t4 each has a duration shorter than 10% of a duration of the first or second time period T1, T2.

According to another aspect of the embodiment of the present disclosure, the first AC signal is applied to the first pair of the insulated electrodes to generate a first electric field between the first pair of the insulated electrodes, and the second AC signal is applied to the second pair of the insulated electrodes to generate a second electric field between the second pair of the insulated electrodes.

According to another aspect of the embodiment of the present disclosure, the first electric field has a direction which is perpendicular to that of the second electric field.

According to another aspect of the embodiment of the present disclosure, the periodic control signal is a periodic square-wave signal.

According to another aspect of the embodiment of the present disclosure, both the first AC signal and the second AC signal have a field strength of at least 1 V/cm.

According to another aspect of the embodiment of the present disclosure, the insulated electrode is configured to apply the alternating electric signal to a tumor site in the torso of the patient during tumor electric field therapy and includes a plurality of electrode elements arranged in an array and having at least ten electrode elements, a plurality of connecting portions each connected with two adjacent electrode elements and a wire electrically connected to the plurality of electrode elements, and the electrode elements are distributed in the array with at least three rows and four columns, and each electrode element is connected with at least two adjacent electrode elements, and at least two adjacent electrode elements among the plurality of electrode elements are arranged in spaced rows or in spaced columns.

According to another aspect of the embodiment of the present disclosure, at least two adjacent electrode elements among the plurality of electrode elements are arranged in a disconnected manner to form a gap located therebetween.

According to another aspect of the embodiment of the present disclosure, the insulated electrode further includes a wiring portion electrically connected to the connecting portion or the electrode element, and the wiring portion is welded to the wire after passing through the gap.

According to another aspect of the embodiment of the present disclosure, all two adjacent electrode elements arranged in rows are arranged in spaced columns, and two adjacent electrode elements in at least the same column among the plurality of electrode elements arranged in columns are arranged in a spaced row.

According to another aspect of the embodiment of the present disclosure, all adjacent two electrode elements arranged in rows have the same distance, and all adjacent two electrode elements arranged in columns have different distances.

According to another aspect of the embodiment of the present disclosure, a plurality of the connecting portions located between a plurality of two adjacent electrode elements arranged in rows have the same length, and a plurality of the connecting portions located between a plurality of two adjacent electrode elements arranged in columns have different lengths.

According to another aspect of the embodiment of the present disclosure, the insulated electrode has 13 electrode elements distributed in a region defined by five rows and five columns.

According to another aspect of the embodiment of the present disclosure, at least two adjacent electrode elements among a plurality of electrode elements arranged in rows are arranged in a spaced column, and all the electrode elements arranged in columns are arranged in adjacent rows.

According to another aspect of the embodiment of the present disclosure, all two adjacent electrode elements arranged in rows have different distances, and all two adjacent electrode elements arranged in columns have the same distance.

According to another aspect of the embodiment of the present disclosure, a plurality of the connecting portions located between a plurality of the adjacent electrode elements arranged in rows have different lengths, and a plurality of the connecting portions located between a plurality of the adjacent electrode elements arranged in columns have the same length.

According to another aspect of the embodiment of the present disclosure, the insulated electrode has 13 electrode elements distributed in a region defined by three rows and five columns.

According to another aspect of the embodiment of the present disclosure, the connecting portion includes a first connecting portion connected with two adjacent electrode elements arranged in the same row and a second connecting portion connected with two adjacent electrode elements arranged in the same column.

According to another aspect of the embodiment of the present disclosure, the connecting portion further includes a third connecting portion connected with two adjacent electrode elements arranged diagonally in adjacent rows and adjacent columns.

According to another aspect of the embodiment of the present disclosure, the third connecting portion has a length larger than that of the first connecting portion.

According to another aspect of the embodiment of the present disclosure, the third connecting portion has a length larger than half of that of the first connecting portion.

According to another aspect of the embodiment of the present disclosure, the third connecting portion has a length larger than that of the second connecting portion.

According to another aspect of the embodiment of the present disclosure, the insulated electrode is configured to apply the alternating electric field to a tumor site in the patient's torso during tumor electric field therapy and includes a plurality of electrode elements distributed in a region defined by at least three rows and four columns, a plurality of connecting portions each located between two adjacent electrode elements and a wiring portion electrically connected with all the electrode elements, and each electrode element is connected to at least two adjacent electrode elements through the corresponding connecting portions, and the plurality of electrode elements are spaced with each other to form a plurality of open spaces located therebetween to allow moisture to escape from the patient's body surface, and the insulated electrode has at least 10 electrode elements and at least two adjacent electrode elements are arranged in a disconnected manner.

According to another aspect of the embodiment of the present disclosure, the plurality of electrode elements are distributed in a region of 109 mm×109 mm to 219 mm×163 mm.

According to another aspect of the embodiment of the present disclosure, in the plurality of the open spaces, a largest open space has an area of 1065 mm² and a smallest open space has an area of 470 mm².

According to another aspect of the embodiment of the present disclosure, two adjacent electrode elements arranged in the same row has a distance about 23 mm to 50 mm.

According to another aspect of the embodiment of the present disclosure, two adjacent electrode elements arranged in the same row has a distance of 23 mm.

According to another aspect of the embodiment of the present disclosure, each connecting portion connected with two electrode elements arranged in the same column and in adjacent rows has a length about 1 mm to 28 mm.

According to another aspect of the embodiment of the present disclosure, each connecting portion connected with two electrode elements arranged in the same column and in adjacent rows has a length about 15 mm.

According to another aspect of the embodiment of the present disclosure, each connecting portion has a width of 4.5 mm to 6 mm.

According to another aspect of the embodiment of the present disclosure, the wiring portion is located between two adjacent electrode elements arranged in the disconnected manner and has a width of 4 mm to 8 mm.

According to another aspect of the embodiment of the present disclosure, the wiring portion extends from an electrode element located at a periphery of the array toward a space formed between two electrode elements arranged in the disconnected manner.

According to another aspect of the embodiment of the present disclosure, the insulated electrode further includes a reinforcing portion disposed opposite to the wiring portion, the reinforcing portion and the wiring portion are respectively located at two opposite sides of the connecting portion which extends to form the wiring portion.

According to another aspect of the embodiment of the present disclosure, the insulated electrode is arranged at a corresponding position of a tumor site in a patient's torso and includes an electrical functional component for applying the alternating electric field to the tumor site of the patient, and the electrical functional component includes a plurality of electrode elements arranged in at least three rows and four columns, a plurality of connecting portions each electrically connected with two adjacent electrode elements and a wiring portion extending from one connecting portion, and a plurality of the connecting portions connected with two adjacent electrode elements arranged in rows or in columns have different lengths.

According to another aspect of the embodiment of the present disclosure, the connecting portions include a first connecting portion extending laterally to form the wiring portion and a plurality of second connecting portions each only connected with two adjacent electrode elements in the same row or in the same column, and each of the first connecting portion is connected with two adjacent electrode elements arranged in the same row but in spaced columns or in the same column but in spaced rows.

According to another aspect of the embodiment of the present disclosure, the second connecting portion connected with two adjacent electrode elements located in the same row and adjacent columns has a length about 1 mm to 3 mm.

According to another aspect of the embodiment of the present disclosure, the first connecting portion has a length about 22 mm to 27 mm.

According to another aspect of the embodiment of the present disclosure, the insulated electrode has 14 the electrode elements arranged in three rows and five columns.

According to another aspect of the embodiment of the present disclosure, a distance formed between two adjacent electrode elements located in the same row and adjacent columns is about 1 mm to 3 mm.

According to another aspect of the embodiment of the present disclosure, a distance formed between two adjacent electrode elements in the same column and adjacent rows is about 1 mm to 3 mm.

According to another aspect of the embodiment of the present disclosure, the insulated electrode includes an electrical functional component for applying the alternating electric field to a torso of a patient, and the electrical functional component includes at least 10 electrode elements arranged in at least three rows and four columns, a plurality of connecting portions connected with two adjacent electrode elements and a wiring portion connected to the connecting portion, and each electrode element is connected with at least two connecting portions, and numbers of the electrode elements in rows or columns are not exactly the same.

According to another aspect of the embodiment of the present disclosure, the insulated electrode has 20 electrode elements distributed in an array surrounded by four rows and six columns.

According to another aspect of the embodiment of the present disclosure, at least two adjacent electrode elements in the same row or in the same column are arranged in a disconnected manner.

According to another aspect of the embodiment of the present disclosure, the electrical functional component has a gap formed between two adjacent electrode elements arranged in a disconnected manner, and the wiring portion passes through the gap.

According to another aspect of the embodiment of the present disclosure, the wiring portion extends from the connecting portion toward the gap.

According to another aspect of the embodiment of the present disclosure, the wiring portion is arranged perpendicularly to the connecting portion, and the wiring portion is arranged substantially in a shape of“−”.

According to another aspect of the embodiment of the present disclosure, the wiring portion bridges two connecting portions which are respectively connected to two adjacent electrode elements arranged in the disconnected manner.

According to another aspect of the embodiment of the present disclosure, the wiring portion is arranged substantially in a shape of “T”.

According to another aspect of the embodiment of the present disclosure, a plurality of two adjacent electrode elements arranged in rows have the same distance formed therebetween, and a plurality of the connecting portions each connected with two adjacent electrode elements arranged in rows have the same length.

According to another aspect of the embodiment of the present disclosure, a plurality of two adjacent electrode elements arranged in columns have the same distance formed therebetween, and a plurality of the connecting portions each connected with two adjacent electrode elements arranged in columns have the same length.

According to another aspect of the embodiment of the present disclosure, at least two adjacent electrode elements arranged in rows are arranged in spaced columns, and distances between a plurality of two adjacent electrode elements arranged in rows are not exactly same.

According to another aspect of the embodiment of the present disclosure, at least two adjacent electrode elements arranged in columns are arranged in spaced rows, and distances between a plurality of two adjacent electrode elements arranged in columns are not exactly same.

According to another aspect of the embodiment of the present disclosure, two adjacent electrode elements located in rows are arranged in adjacent columns, and distances between a plurality of two adjacent electrode elements arranged in rows are the same.

According to another aspect of the embodiment of the present disclosure, two adjacent electrode elements located in columns are arranged in adjacent rows, and distances between a plurality of two adjacent electrode elements arranged in columns are the same.

According to another aspect of the embodiment of the present disclosure, distances formed between a plurality of two adjacent electrode elements arranged in rows are the same, and distances formed between a plurality of two adjacent electrode elements arranged in columns are the same.

According to another aspect of the embodiment of the present disclosure, the plurality of the electrode elements are distributed in the array defined by four rows and six columns, and two electrode elements are arranged in each of a first and last columns, and four electrode elements are arranged in each of middle four columns.

According to another aspect of the embodiment of the present disclosure, the plurality of electrode elements are arranged in an axisymmetric manner both along a row direction and a column direction.

According to another aspect of the embodiment of the present disclosure, the insulated electrode further includes a wire electrically connected to the electrical functional component, and the wire is welded to the wiring portion.

According to another aspect of the embodiment of the present disclosure, the insulated electrode further includes an adhesive-backed layer supporting the electrical functional component, and the adhesive-backed layer includes an aperture for the wire passing through.

According to another aspect of the embodiment of the present disclosure, the insulated electrode includes a plurality of electrode elements distributed in at least three rows and four columns, a plurality of connecting portions each connected with two adjacent electrode elements and a wiring portion connected to the connecting portion, and each electrode element is connected with at least two connecting portions, and the plurality of electrode elements are arranged at intervals to form a plurality of open spaces located therebetween, and the wiring portion passes through one open space.

According to another aspect of the embodiment of the present disclosure, two adjacent electrode elements arranged in the same row has a distance about 8 mm to 22 mm.

According to another aspect of the embodiment of the present disclosure, two adjacent electrode elements arranged in the same column has a distance about 6.5 mm to 25 mm.

According to another aspect of the embodiment of the present disclosure, a distance between two adjacent electrode elements arranged diagonally in adjacent rows and adjacent columns is 19 mm to 33.3 mm.

According to another aspect of the embodiment of the present disclosure, the plurality of electrode elements are distributed at intervals in a region of 166 mm×103.5 mm to 242 mm×166 mm.

According to another aspect of the embodiment of the present disclosure, each connecting portion has a width of 4.5 mm to 6 mm, or preferably, the width of the connecting portion is 4.5 mm.

According to another aspect of the embodiment of the present disclosure, at least two adjacent electrode elements in the same row or the same column among the plurality of electrode elements are arranged in a disconnected manner and a gap is formed between the two adjacent electrode elements arranged in the disconnected manner.

According to another aspect of the embodiment of the present disclosure, the wiring portion passes through the gap and includes a bridge section bridging between two connecting portions and a wiring section extended from the bridge section toward the gap.

According to another aspect of the embodiment of the present disclosure, the bridge section of the wiring portion has a width about 4.5 mm to 6 mm, and the wiring section of the wiring portion has a width about 4 mm to 8 mm.

According to another aspect of the embodiment of the present disclosure, the insulated electrode has 20 electrode elements distributed in an array surrounded by four rows and six columns, and among the plurality of the open spaces, a largest open space has an area of 4428 mm² and a smallest open space has an area of 452 mm².

According to another aspect of the embodiment of the present disclosure, the insulated electrode includes an electrical functional component for applying the alternating electric signal to a tumor site of a patient and a wire electrically connected to the electrical functional component, and the electrical functional component includes at least 10 electrode elements arranged at intervals, a plurality of connecting portions each connected with two adjacent electrode elements and a wiring portion electrically connected to the wire, and each electrode element is connected with at least two connecting portions.

According to another aspect of the embodiment of the present disclosure, each electrode element is connected to at least two electrode elements adjacent thereto.

According to another aspect of the embodiment of the present disclosure, the electrode elements are distributed in an array with at least three rows and four columns, and the insulated electrode has at least 10 but at most 30 electrode elements.

According to another aspect of the embodiment of the present disclosure, the electrode elements are distributed in an array with at least three rows and four columns, and the electrode elements in each row have the same number and are arranged in a column-alignment manner.

According to another aspect of the embodiment of the present disclosure, the electrode elements are arranged in the same row spacing manner.

According to another aspect of the embodiment of the present disclosure, the electrode elements are arranged in the same column spacing manner.

According to another aspect of the embodiment of the present disclosure, the connecting portions each connected with two adjacent electrode elements arranged in rows have the same length.

According to another aspect of the embodiment of the present disclosure, the connecting portions each connected with two adjacent electrode elements arranged in columns have the same length.

According to another aspect of the embodiment of the present disclosure, at least two adjacent electrode elements in the same row or the same column among the plurality of electrode elements are arranged in a disconnected manner and a gap allowing the wiring portion to pass through is formed between the two adjacent electrode elements arranged in the disconnected manner.

According to another aspect of the embodiment of the present disclosure, the wiring portion extends laterally from the connecting portion which is opposite to the gap.

According to another aspect of the embodiment of the present disclosure, the connecting portion that extends to form the wiring portion is perpendicular to the wiring portion.

According to another aspect of the embodiment of the present disclosure, the wiring portion bridges two connecting portions respectively connected to two electrode elements arranged in the disconnected manner.

According to another aspect of the embodiment of the present disclosure, the wiring portion is arranged substantially in a shape of “T”.

According to another aspect of the embodiment of the present disclosure, the insulated electrode has 20 electrode elements distributed in an array with four rows and five columns.

According to another aspect of the embodiment of the present disclosure, the electrode element includes a main body arranged at an end of the connecting portion, an insulating plate arranged on a side of the main body that is further away from human skin and a dielectric element arranged on a side of the main body that faces toward the human skin.

According to another aspect of the embodiment of the present disclosure, the electrode element further includes a temperature sensor selectively arranged on the main body, and the temperature sensor and the dielectric element are located on the same side of the main body.

According to another aspect of the embodiment of the present disclosure, the dielectric element has a through-hole corresponding to the temperature sensor.

According to another aspect of the embodiment of the present disclosure, the insulated electrode further includes an adhesive-backed layer that supports the electrical functional component, and the adhesive-backed layer has an aperture allowing the wire to pass through.

According to another aspect of the embodiment of the present disclosure, the wire has a heat shrink sleeve which wraps around a joint between the wire and the wiring portion.

According to another aspect of the embodiment of the present disclosure, the insulated electrode includes a flexible circuit board, a dielectric element, a plurality of temperature sensors and a wire electrically connected to the flexible circuit board, and the dielectric element and the temperature sensors are located on the same side of the flexible circuit board, and the number of temperature sensors is n, n is an integer greater than 1 but not greater than 8, and each temperature sensor has a ground terminal and a signal terminal, and the flexible circuit board has an insulating substrate and n+2 conductive traces embedded in the insulating substrate, and one conductive trace is electrically connected to the dielectric element, and one conductive trace is electrically connected to all the ground terminals of all the temperature sensors, and the rest conductive traces are respectively and electrically connected to corresponding signal terminal of corresponding temperature sensor, and the wire is electrically connected with all the conductive traces of the flexible circuit board.

According to another aspect of the embodiment of the present disclosure, the flexible circuit board has a plurality of golden fingers exposed from the insulating substrate thereof, and the plurality of golden fingers are respectively connected to corresponding portions of the wire.

According to another aspect of the embodiment of the present disclosure, each golden finger is electrically connected to one conductive trace of the flexible circuit board.

According to another aspect of the embodiment of the present disclosure, the insulated electrode has two temperature sensors, and the insulating plate has four conductive traces, and the flexible circuit has four golden fingers.

According to another aspect of the embodiment of the present disclosure, the flexible circuit has a conductive pad corresponding to the dielectric element and the conductive pad is welded with the dielectric element.

According to another aspect of the embodiment of the present disclosure, the conductive pad is exposed from the insulating substrate and is connected to one conductive trace that electrically connects the flexible circuit board to the dielectric element.

According to another aspect of the embodiment of the present disclosure, the conductive pad includes a plurality of conductive cores arranged at intervals, and the plurality of conductive cores are connected in series with the conductive trace which electrically connects the flexible circuit board to the dielectric element.

According to another aspect of the embodiment of the present disclosure, the flexible circuit board has n pairs of bonding pads, and each pair of the bonding pads are located between two corresponding conductive cores arranged at intervals.

According to another aspect of the embodiment of the present disclosure, each pair of the bonding pads are arranged at corresponding positions of the flexible circuit board corresponding to the temperature sensor, and each pair of the bonding pads are exposed from the insulating substrate of the flexible circuit board.

According to another aspect of the embodiment of the present disclosure, each pair of the bonding pads include a first bonding pad and a second bonding pad, and the first bonding pad is welded to the ground terminal of the corresponding temperature sensor, and the second bonding pad is welded to the signal terminal of the corresponding temperature sensor.

According to another aspect of the embodiment of the present disclosure, the first bonding pad is connected to the conductive trace which electrically connects the flexible circuit board to the ground terminal of the temperature sensor, and each second bonding pads is connected to one conductive trace that electrically connects the flexible circuit board to the signal terminal of the corresponding temperature sensor.

According to another aspect of the embodiment of the present disclosure, the wire has one end electrically connected to the flexible circuit board and the other end having a plug.

According to another aspect of the embodiment of the present disclosure, a heat shrink sleeve is provided at a joint between the wire and the flexible circuit board.

According to another aspect of the embodiment of the present disclosure, the dielectric element has a through-hole corresponding to the temperature sensor, and the temperature sensor is accommodated in the corresponding through-hole.

According to another aspect of the embodiment of the present disclosure, among the plurality of conductive traces, the conductive trace electrically connected with the dielectric element is a first conductive trace, and the conductive trace electrically connected with the ground terminals of the temperature sensors is a second conductive trace, and the rest n conductive traces electrically connected to the signal terminals of the corresponding temperature sensors respectively are all third conductive traces, and the flexible circuit board has a conductive pad connected with the first conductive trace and n pairs of bonding pads, and each pair of the bonding pads includes one bonding pad connected to the second conductive trace and the other one connected to the corresponding third conductive trace.

According to another aspect of the embodiment of the present disclosure, the conductive pad and the bonding pads are located on the same side of the flexible circuit board.

According to another aspect of embodiment of the present disclosure, both the conductive pad and the bonding pads are exposed from the insulating substrate of the flexible circuit board.

According to another aspect of the embodiment of the present disclosure, the flexible circuit board further includes n golden fingers welded with the wire and exposed from the insulating substrate thereof, and n is equal to n plus two, and n is an integer greater than 1 and not greater than 8.

According to another aspect of the embodiment of the present disclosure, the flexible circuit board has 4 golden fingers, two pairs of the bonding pads and two third conductive traces, and the insulated electrode has two temperature sensors.

According to another aspect of the embodiment of the present disclosure, the golden fingers, the conductive pad and the two pairs of bonding pads are all located on the same side of the flexible circuit board.

According to another aspect of the embodiment of the present disclosure, the insulated electrode also includes an adhesive-backed layer adhered to a corresponding portion of the flexible circuit board.

According to another aspect of the embodiment of the present disclosure, the insulated electrode also includes an insulating plate arranged on a side of the flexible circuit board that is away from the dielectric element, and the insulating plate corresponds to the dielectric element along a thickness direction, and the insulating plate is sandwiched between the flexible circuit board and the adhesive-backed layer.

According to another aspect of the embodiment of the present disclosure, the insulated electrode includes at least one electrode segment for applying the alternating electric signal and an electrical connector detachably connected to the electrode segment, and the electrode segment includes a single electrode element and a first wire electrically connected to the electrode element, and the electrode segment is detachably connected to the electrical connector through the first wire.

According to another aspect of the embodiment of the present disclosure, a plurality of electrode segments are connected to the electrical connector in parallel through the corresponding first wires thereof.

According to another aspect of the embodiment of the present disclosure, the first wire of the electrode segment has a first plug detachably plugged into the electrical connector, and the first plug and the electrode element are respectively located at opposite ends of the first wire.

According to another aspect of the embodiment of the present disclosure, the electrical connector has a plurality of sockets into which the first plug of the first wire of the corresponding electrode segment can be detachably plugged.

According to another aspect of the embodiment of the present disclosure, the electrical connector includes a second wire, the second wire and the plurality of sockets are respectively located at opposite ends of the electrical connector.

According to another aspect of the embodiment of the present disclosure, the second wire has a second plug provided at an end thereof.

According to another aspect of the embodiment of the present disclosure, the electrical connector has a body, and the plurality of sockets and the second wire are respectively provided at opposite ends of the body.

According to another aspect of the embodiment of the present disclosure, the electrode segment further includes a wiring portion connected to the electrode element, and the wiring portion is welded to an end of the first wire that is away from the first plug.

According to another aspect of the embodiment of the present disclosure, the electrode element includes a main body and a dielectric element welded to one side of the main body, and the wiring portion extends laterally from the main body.

According to another aspect of the embodiment of the present disclosure, the main body of the electrode element and the wiring portion form a flexible circuit board of the electrode segment.

According to another aspect of the embodiment of the present disclosure, the electrode element further includes at least one temperature sensor, and the temperature sensor is located on the main body and on the same side as the dielectric element.

According to another aspect of the embodiment of the present disclosure, the dielectric element has at least one through-hole located in the middle thereof, and the temperature sensors are respectively accommodated in corresponding through-holes of the dielectric element.

According to another aspect of the embodiment of the present disclosure, the electrode element further includes an insulating plate adhered to one side of the main body that is away from the dielectric element.

According to another aspect of the embodiment of the present disclosure, a heat shrink sleeve is wrapped around a welded joint of the first wire and the wiring portion.

According to another aspect of the embodiment of the present disclosure, the first wire is detachably connected to the electrode element.

According to another aspect of the embodiment of the present disclosure, the electrode segment includes a wiring portion electrically connected to the electrode element, and the wiring portion has a docking socket located at one end thereof that is away from the electrode element.

According to another aspect of the embodiment of the present disclosure, the first wire has a docking plug located at one end thereof which is away from the first plug, and the docking plug is detachably plugged into the docking socket.

According to another aspect of the embodiment of the present disclosure, the electrode segment further includes an adhesive-backed layer adhered to the electrode element, a supporting member surrounding the electrode element and adhered to the adhesive-backed layer and an adhesive member covering sides of the electrode element and the supporting member that are away from the adhesive-backed layer.

According to another aspect of the embodiment of the present disclosure, the insulated electrode includes an adhesive-backed layer, a flexible circuit board arranged on the adhesive-backed layer, a dielectric element and a heat dissipation reinforcing member, the heat dissipation reinforcing member and the dielectric element are respectively arranged on opposite sides of the flexible circuit board, the heat dissipation reinforcing member is sandwiched between the flexible circuit board and the adhesive-backed layer, and the heat dissipation reinforcing member is made of a material with a thermal conductivity greater than 200 W/mK.

According to another aspect of the embodiment of the present disclosure, the heat dissipation reinforcing member has at least one heat dissipation hole.

According to another aspect of the embodiment of the present disclosure, the heat dissipation reinforcing member is a metal plate, or a metal alloy plate or a graphene composite plate.

According to another aspect of the embodiment of the present disclosure, the heat dissipation reinforcing member is a metal plate or metal alloy plate having a thickness of 0.1 mm to 0.7 mm.

According to another aspect of the embodiment of the present disclosure, the heat dissipation reinforcing member is an aluminium plate or aluminium alloy plate having a thickness of 0.3 mm to 0.6 mm.

According to another aspect of the embodiment of the present disclosure, the heat dissipation reinforcing member is an aluminum plate having a thickness of 0.6 mm.

According to another aspect of the embodiment of the present disclosure, 30 heat dissipation holes are evenly distributed on the aluminum plate and each heat dissipation hole has a diameter of 0.5 mm.

According to another aspect of the embodiment of the present disclosure, the heat dissipation reinforcing member is an aluminum alloy plate having a thickness of 0.3 mm and a thermal conductivity of 201 W/mK.

According to another aspect of the embodiment of the present disclosure, 50 heat dissipation holes are evenly distributed on the aluminum alloy plate and each heat dissipation hole has a diameter of 0.4 mm.

According to another aspect of the embodiment of the present disclosure, the heat dissipation reinforcing member is a graphene composite plate having a thickness of 0.1 mm and a thermal conductivity greater than 300 W/mK.

According to another aspect of the embodiment of the present disclosure, the insulated electrode includes a flexible adhesive-backed layer and an electrical functional component supported by the flexible adhesive-backed layer, the electrical functional component includes a dielectric element closer to a body surface of a patient that corresponds to a target region and a heat dissipation reinforcing member corresponding to the dielectric element, the heat dissipation reinforcing member is sandwiched between the dielectric element and the flexible adhesive-backed layer, and the heat dissipation reinforcing member is made of a material with a thermal conductivity greater than 200 W/mK.

According to another aspect of the embodiment of the present disclosure, the heat dissipation reinforcing member is located on one side of the dielectric element that is away from the body surface of the patient's target region and has at least one heat dissipation hole penetrating therethrough.

According to another aspect of the embodiment of the present disclosure, the heat dissipation reinforcing member is a metal plate or metal alloy plate having a thickness of 0.1 mm to 0.7 mm.

According to another aspect of the embodiment of the present disclosure, the heat dissipation reinforcing member is an aluminium plate or aluminium alloy plate having a thickness of 0.3 mm to 0.6 mm.

According to another aspect of the embodiment of the present disclosure, the heat dissipation reinforcing member is an aluminum plate having a thickness of 0.6 mm, and 30 heat dissipation holes are evenly distributed on the aluminum plate, and each heat dissipation hole has a diameter of 0.5 mm.

According to another aspect of the embodiment of the present disclosure, the heat dissipation reinforcing member is an aluminum alloy plate with a thickness of 0.3 mm and a thermal conductivity of 201 W/mK, and 50 heat dissipation holes are evenly distributed on the aluminum alloy plate, and each heat dissipation hole has a diameter of 0.4 mm.

According to another aspect of the embodiment of the present disclosure, the heat dissipation reinforcing member is a graphene composite plate with a thickness of 0.1 mm and a thermal conductivity greater than 300 W/mK.

According to another aspect of the embodiment of the present disclosure, the insulated electrode also includes a supporting member adhered to the flexible adhesive-backed layer and surrounding the dielectric element and an adhesive member covering the supporting member and the dielectric element, the electrical functional component further includes a temperature sensor contacting with the adhesive member to detect temperature of the adhesive member, and the heat dissipation reinforcing member is electrically insulated from the dielectric element.

According to another aspect of the embodiment of the present disclosure, the insulated electrode includes an electrical functional component having a flexible circuit board, a dielectric element and a temperature sensor, and the dielectric element and the temperature sensor are both arranged on the flexible circuit board's one side that faces toward the skin of the patient, and the electrical functional component further includes a semiconductor refrigerator arranged on the flexible circuit board's the other side that faces away from the dielectric element, and the semiconductor refrigerator is arranged corresponding to the dielectric element along a thickness direction of the flexible circuit board.

According to another aspect of the embodiment of the present disclosure, the semiconductor refrigerator includes a refrigeration end closer to the flexible circuit board and a heat dissipation end opposite to the refrigeration end, and the refrigeration end is electrically connected to the flexible circuit board.

According to another aspect of the embodiment of the present disclosure, the semiconductor refrigerator further includes an N-type semiconductor and a P-type semiconductor both sandwiched side by side between the refrigeration end and the heat dissipation end.

According to another aspect of the embodiment of the present disclosure, the semiconductor refrigerator further includes a sealant filled between the refrigeration end and the heat dissipation end to seal the N-type semiconductor and the P-type semiconductor.

According to another aspect of the embodiment of the present disclosure, the refrigeration end of the semiconductor refrigerator has welding pads soldered to the flexible circuit board, and the welding pads includes a positive welding pad and a negative welding pad.

According to another aspect of the embodiment of the present disclosure, the refrigeration end of the semiconductor refrigerator includes a cold-end ceramic plate arranged on the side of the flexible circuit board that faces away from the skin of the patient, a cold-end thermal conduction member arranged on the cold-end ceramic plate and two cold-end metal conductors arranged on the cold-end thermal conduction member at intervals, and the two cold-end metal conductors are respectively connected to the N-type semiconductor and the P-type semiconductor.

According to another aspect of the embodiment of the present disclosure, the welding pads are located on the cold-end ceramic plate's one side that faces toward the flexible circuit board, and the flexible circuit board has a welding portion corresponding to the welding pads of the cold-end ceramic plate, and the welding portion is connected to the welding pad to achieve an electrical connection between the cold-end ceramic plate and the flexible circuit board.

According to another aspect of the embodiment of the present disclosure, the cold-end ceramic plate has conductive traces that are electrically connected to the positive welding pad and the negative welding pad respectively, and the cold-end thermal conduction member has conductive traces corresponding to the conductive traces of the cold-end ceramic plate, and the two cold-end metal conductors are electrically connected to the positive welding pad and the negative welding pad of the cold-end ceramic plate through corresponding conductive traces of the cold-end thermal conduction member and the cold-end ceramic plate.

According to another aspect of the embodiment of the present disclosure, the heat dissipation end of the semiconductor refrigerator includes a hot-end ceramic plate, a hot-end thermal conduction member arranged on the hot-end ceramic plate's one side that is closer to the refrigeration end and a hot-end metal conductor arranged on the hot-end thermal conduction member and contacting with the N-type semiconductor and the P-type semiconductor.

According to another aspect of the embodiment of the present disclosure, the N-type semiconductor and the P-type semiconductor are sandwiched between the hot-end metal conductor and the two cold-end metal conductors.

According to another aspect of the embodiment of the present disclosure, the N-type semiconductor, the P-type semiconductor, the cold-end metal conductors and the hot-end metal conductor are all made of the same material.

According to another aspect of the embodiment of the present disclosure, the positive welding pad of the cold-end ceramic plate of the semiconductor refrigerator is electrically conductive to the N-type semiconductor, and the negative welding pad of the cold-end ceramic plate is electrically conductive to the P-type semiconductor.

According to another aspect of the embodiment of the present disclosure, the temperature sensor and the semiconductor refrigerator are respectively arranged on opposite sides of the flexible circuit board, and the dielectric element has a through-hole and the temperature sensor is accommodated in the through-hole.

According to another aspect of the embodiment of the present disclosure, the insulated electrode also includes an adhesive-backed layer, and the electrical functional component is attached to the adhesive-backed layer through the hot-end ceramic plate of the semiconductor refrigerator, and the semiconductor refrigerator is sandwiched between the adhesive-backed layer and the flexible circuit board.

According to another aspect of the embodiment of the present disclosure, the insulated electrode also includes a supporting member surrounding the dielectric element and an adhesive member covering the supporting member and the dielectric element, and the temperature sensor contacts with the adhesive member and monitors temperature of the adhesive member.

According to another aspect of the embodiment of the present disclosure, the adhesive-backed layer has an opening located at a position thereof corresponding to the hot-end ceramic plate of the semiconductor refrigerator to expose the hot-end ceramic plate to the air.

According to another aspect of the embodiment of the present disclosure, the tumor electric field therapy system further includes a controller electrically connected to the insulated electrode, and the controller controls the turn-on or turn-off of the semiconductor refrigerator of the insulated electrode or the turn-off of the tumor electric field therapy system by monitoring temperature of the insulated electrode.

According to another aspect of the embodiment of the present disclosure, a temperature control method applied to the aforementioned tumor electric field therapy system including: S1: monitoring the temperature of the insulated electrode in real time; S2: determining whether the monitored temperature exceeds a regulating temperature or not; S3: according to a determining result in step S2, turning off the semiconductor refrigerator of the insulated electrode or continuing to determine whether the monitored temperature exceeds a safety threshold or not; and S4: according to a determining result of whether the temperature exceeding a safety threshold or not in step S3, turning on the semiconductor refrigerator of the insulated electrode or turning off the tumor electric field therapy system.

According to another aspect of the embodiment of the present disclosure, monitoring the temperature of the insulated electrode in real time is implemented by the temperature sensor.

According to another aspect of the embodiment of the present disclosure, the safety threshold is greater than the regulating temperature.

According to another aspect of the embodiment of the present disclosure, a difference between the safety threshold and the regulating temperature is within 4° C.

According to another aspect of the embodiment of the present disclosure, the regulating temperature is 390 and the safety threshold is 41°.

According to another aspect of the embodiment of the present disclosure, the turning off the semiconductor refrigerator of the insulated electrode or continuing to determine whether the monitored temperature exceeding a safety threshold or not according to a determining result in step S2 specifically includes the following steps:
when the monitored temperature is lower than the regulating temperature, controlling the semiconductor refrigerator of the insulated electrode to enter a turn-off state; and
when the monitored temperature is higher than or equal to the regulating temperature, determining whether the monitored temperature exceeds the safety threshold or not.

According to another aspect of the embodiment of the present disclosure, the determining result in step S2 comprises the monitored temperature being lower than the regulating temperature and the monitored temperature being higher than or equal to the regulating temperature.

According to another aspect of the embodiment of the present disclosure, the determining result of the monitored temperature whether exceeding the safety threshold or not in step S3 comprises the monitored temperature being lower than the safety threshold and the monitored temperature being higher than or equal to the safety threshold.

According to another aspect of the embodiment of the present disclosure, the turning on the semiconductor refrigerator of the insulated electrode or turning off the tumor electric field therapy system according to a determining result of whether the temperature exceeding a safety threshold or not in step S3 specifically includes the following steps: when the monitored temperature is lower than the safety threshold, controlling the semiconductor refrigerator of the insulated electrode to enter a turn-on state and performing step S1 to step S3 repeatedly and periodically; and when the monitored temperature is higher than or equal to the safety threshold, controlling the tumor electric field therapy system to enter a turn-off state.

According to another aspect of the embodiment of the present disclosure, the turning on of the semiconductor refrigerator of the insulated electrode is implemented by supplying a direct current to the semiconductor refrigerator.

The AC signal generator of the electric field treatment device of the tumor electric field therapy system for applying an alternating electric signal to the tumor in the torso according to the present disclosure is configured such that when alternately applying alternating electric signals to different pairs of insulated electrodes, the AC voltage value of the alternating electric signal applied to the insulated electrodes arranged in pair rises from 0 to a specific voltage at a constant speed during a switching-on time period t3 with a preset value or decreases from a specific value to 0 at a constant speed during a switching-off time period t4 with a preset value; and the AC voltage change per millisecond in the switching-on time period t3 and the switching-off time period t4 are both within 5% of the specific voltage. Accordingly, not only a damage of the AC signal controller caused by a sharp change in the alternating electric signal applied to the different pairs of insulated electrodes during direction switching can be prevented, but also a tingling sensation in experimental animals or humans caused by a sharp change in the AC voltage of the insulated electrodes arranged on the body surface corresponding to the tumor site is avoided.

It should be understood that the aforementioned general descriptions and the following detailed descriptions are merely illustrative and explanatory, and do not constitute any limitation on the present disclosure.

### BRIEF DESCRIPTION OF THE DRAWINGS

To more clearly illustrate the technical solutions in the embodiments of the present disclosure, the following briefly describes the accompanying drawings required for describing the embodiments. It is evident that the accompanying drawings in the following description are just some embodiments of the present disclosure, and a person of ordinary skill in the art can also derive additional drawings from these accompanying drawings without creative efforts.

FIG. 1 is a systematic block diagram of a tumor electric field therapy system according to the present disclosure;

FIG. 2 is a schematic diagram of control signals for turning on or off a first electric field and a second electric field in a tumor electric field therapy system according to the present disclosure;

FIG. 3 is a schematic diagram of an AC signal applied to an insulated electrode;

FIG. 4 shows a relationship between a cell growth rate and an electric field operating period;

FIG. 5 is a three-dimensional assembly diagram of a first embodiment of an insulated electrode of a tumor electric field therapy system according to the present disclosure;

FIG. 6 is a three-dimensional exploded view of the insulated electrode shown in FIG. 5;

FIG. 7 is a three-dimensional exploded view of an electrical functional component of the insulated electrode shown in FIG. 6;

FIG. 8 is a planar diagram of a dielectric element of the electrical functional component shown in FIG. 7;

FIG. 9 is a planar diagram of a flexible circuit board of the insulated electrode shown in FIG. 6;

FIG. 10 is a three-dimensional assembly diagram of a variant embodiment of an insulated electrode in the first embodiment of the tumor electric field therapy system according to the present disclosure;

FIG. 11 is a three-dimensional exploded view of an electrical functional component of the insulated electrode shown in FIG. 10;

FIG. 12 is a three-dimensional assembly diagram of a second embodiment of an insulated electrode in the tumor electric field therapy system according to the present disclosure;

FIG. 13 is a three-dimensional diagram of a flexible circuit board and a wire of the insulated electrode shown in FIG. 12;

FIG. 14 is a three-dimensional diagram of an insulated electrode in a third embodiment of a tumor electric field therapy system according to the present disclosure;

FIG. 15 is a three-dimensional exploded view of the insulated electrode shown in FIG. 14;

FIG. 16 is a three-dimensional exploded view of an electrical functional component of the insulated electrode shown in FIG. 15;

FIG. 17 is a three-dimensional assembly diagram of an electrical functional component shown in FIG. 15;

FIG. 18 is a three-dimensional assembly diagram of a fourth embodiment of an insulated electrode of a tumor electric field therapy system according to the present disclosure;

FIG. 19 is a planar bottom view of the insulated electrode shown in FIG. 18;

FIG. 20 is a three-dimensional exploded view of the insulated electrode shown in FIG. 18;

FIG. 21 is a three-dimensional exploded view of an electrical functional component and a wire of the insulated electrode shown in FIG. 20;

FIG. 22 is a planar diagram of a fifth embodiment of an insulated electrode of a tumor electric field therapy system according to the present disclosure;

FIG. 23 is a three-dimensional exploded view of the insulated electrode shown in FIG. 22;

FIG. 24 is a planar diagram of the electrical functional component shown in FIG. 23;

FIG. 25 is a three-dimensional assembly diagram of a sixth embodiment of an insulated electrode of a tumor electric field therapy system according to the present disclosure;

FIG. 26 is a three-dimensional exploded view of the insulated electrode shown in FIG. 25;

FIG. 27 is a three-dimensional exploded view of an electrical functional component and a wire of the insulated electrode shown in FIG. 26;

FIG. 28 is a schematic planar diagram of a flexible circuit board of the insulated electrode shown in FIG. 27;

FIG. 29 is a wiring diagram of a front view of the flexible circuit board of the electrical functional component shown in FIG. 28;

FIG. 30 is a wiring diagram of a back view of the flexible circuit board of the electrical functional component shown in FIG. 28;

FIG. 31 is similar to FIG. 25 and shows a three-dimensional assembly diagram of one variant embodiment of the sixth embodiment of the insulated electrode, and the insulated electrode has an adhesive-backed layer slightly different with that shown in FIG. 25;

FIG. 32 is a three-dimensional exploded view of a variant embodiment of the sixth embodiment of the insulated electrode shown in FIG. 26;

FIG. 33 is a three-dimensional exploded view of an electrical functional component and a wire of the insulated electrode shown in FIG. 32;

FIG. 34 is a schematic planar diagram of a flexible circuit board of the insulated electrode shown in FIG. 33;

FIG. 35 is a three-dimensional assembly diagram of a seventh embodiment of an insulated electrode of a tumor electric field therapy system according to the present disclosure;

FIG. 36 is an exploded view of electrode segments and an electrical connector of the insulated electrode shown in FIG. 35;

FIG. 37 is a three-dimensional exploded view of the electrode segment of the insulated electrode shown in FIG. 36;

FIG. 38 is a three-dimensional exploded view of an electrode element and a first wire of the electrode segment shown in FIG. 37;

FIG. 39 is a planar diagram of a flexible circuit board of the electrode segment shown in FIG. 38;

FIG. 40 is an exploded view of a variant embodiment of an insulated electrode in the seventh embodiment of a tumor electric field therapy system according to the present disclosure;

FIG. 41 is a three-dimensional exploded view of an electrode segment of the insulated electrode shown in FIG. 40;

FIG. 42 is a schematic three-dimensional exploded view of a eighth embodiment of an insulated electrode of a tumor electric field therapy system according to the present disclosure;

FIG. 43 is a schematic three-dimensional exploded view of an electrical functional component of the insulated electrode shown in FIG. 42;

FIG. 44 is a schematic three-dimensional diagram of a heat dissipation reinforcing member of the insulated electrode shown FIG. 43;

FIG. 45 is a schematic three-dimensional exploded view of the insulated electrode shown in FIG. 42;

FIG. 46 is a three-dimensional exploded view of a partial structure of a variant embodiment of an insulated electrode in the eighth embodiment of a tumor electric field therapy system according to the present disclosure;

FIG. 47 is a three-dimensional view of a flexible circuit board of the electrical functional component shown in FIG. 46;

FIG. 48 is a three-dimensional view of a semiconductor refrigerator of the insulated electrode shown in FIG. 47;

FIG. 49 is a cross-sectional view of the flexible circuit board and the semiconductor refrigerator shown in FIG. 48;

FIG. 50 is a flowchart of temperature control steps of a tumor electric field therapy system having the insulated electrode shown in FIG. 46; and

FIG. 51 is a schematic flowchart of temperature control steps of a tumor electric field therapy system having the insulated electrode shown in FIG. 46.

### DETAILED DESCRIPTION

The technical solutions of the embodiments of the present application will be described below clearly and comprehensively in conjunction with the drawings of the embodiments of the present application. Clearly, the embodiments described are merely some embodiments of the present application and are not all the possible embodiments. Based on the embodiments given in the present application, all other embodiments that would be obtained by those of ordinary skill in the art without expending inventive effort shall all fall within the scope of protection of the present application.

Some embodiments are described in detail herein, and some embodiments are presented in the accompanying drawings. When the following description relates to the accompanying drawings, unless specified otherwise, same numbers in different accompanying drawings represent a same or similar element. Implementations described in the following embodiments do not represent all implementations consistent with the present disclosure. On the contrary, they are only embodiments of apparatuses and methods that are described in the appended claims in detail and that are consistent with some aspects of the present disclosure.

The terms used in the present disclosure are merely intended to describe specific embodiments, but not intended to limit the present disclosure. The terms “a/an”, “the” and “this” of singular forms used in the present disclosure and the appended claims may refer to a singular form or may also include plural forms, unless otherwise specified in the context clearly. It should be further understood that, as used herein, the term “and/or” indicates and includes any or all possible combinations of one or more associated listed items. Unless otherwise specified, similar terms such as “connect” or “connection” are not limited to physical or mechanical connections and may include electrical connections, irrespective of direct or indirect connections.

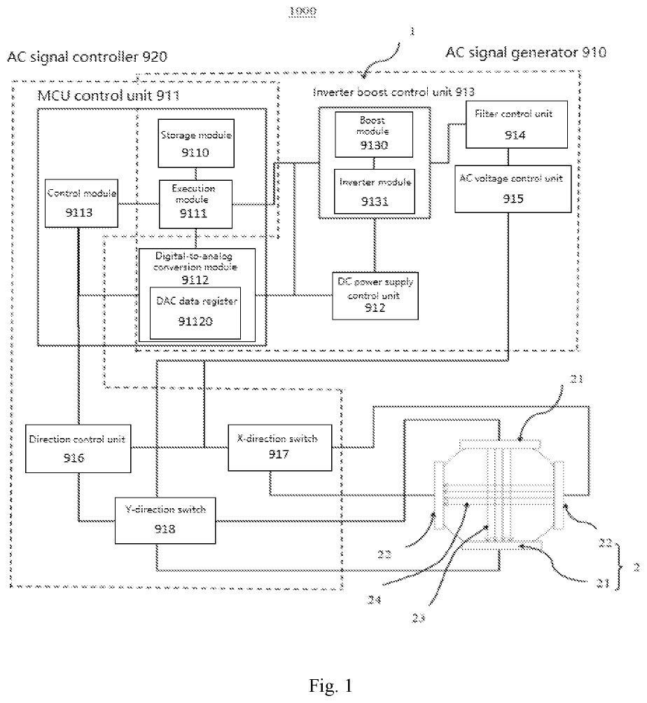

Referring to FIG. 1, a tumor electric field therapy system 1000 in the present invention is used to apply an alternating electrical signal to a tumor site or proliferating cells in tissue culture to perform tumor treatment or inhibit the proliferating cells in the tissue culture, and includes an electric field treatment device 1 and two pairs of insulated electrodes 2 electrically connected to the electric field treatment device 1. The electric field treatment device 1 generates an alternating electrical signal for tumor treatment or inhibition of the proliferating cells in the tissue culture and periodically and alternately applies the generated alternating electrical signal to the two pairs of insulated electrodes 2 to generate alternating electric fields with different directions between the two pairs of insulated electrodes 2. The electric field treatment device 1 includes an electric field generator (not shown in the figure) and an adapter (not shown in the figure) electrically connected to the electric field generator. The two pairs of insulated electrodes 2 include a pair of Y-direction electrodes 21 and a pair of X-direction electrodes 22 that are electrically connected to the electric field treatment device 1. The two Y-direction electrodes 21 are arranged in parallel, and the two X-direction electrodes 22 are arranged in parallel. The two Y-direction electrodes 21 and the two X-direction electrodes 22 are arranged perpendicular to each other. A Y-direction electric field 23 is generated between the two Y-direction electrodes 21. An X-direction electric field 24 is generated between the two X-direction electrodes 22. The X-direction electric field 24 and the Y-direction electric field 23 are perpendicular to each other.

The electric field treatment device 1 includes an MCU control unit 911, an inverter boost control unit 913 electrically connected to the MCU control unit 911, a DC power supply control unit 912 communicated with both the MCU control unit 911 and the inverter boost control unit 913, a filter control unit 914 electrically connected to the inverter boost control unit 913, an AC voltage control unit 915 electrically connected to the filter control unit 914, a direction control unit 916 electrically connected to the MCU control unit 911, an X-direction switch 917 that is electrically connected to the direction control unit 916 and controls connection and disconnection between the AC voltage control unit 915 and two X-direction electrodes 22, and a Y-direction switch 918 that is electrically connected to the direction control unit 916 and controls connection and disconnection between the AC voltage control unit 915 and two Y-direction electrodes 21.

The MCU control unit 911 has a reference voltage of 3.3 Volt and includes a storage module 9110, an execution module 9111 communicated with the storage module 9110, a digital-to-analog conversion module (DAC) 9112 communicated with the execution module 9111, and a control module 9113 that controls the storage module 9110, the execution module 9111 and the digital-to-analog conversion module 9112 to perform respective operations. The storage module 9110 is configured to store preset system parameters of the electric field treatment device 1, the system parameters includes an electric field frequency, an output AC voltage amplitude, a direction switching period of the alternating electric signal, etc.

The execution module 9111 is configured to read the electric field frequency, the output AC voltage amplitude and the direction switching period of the alternating electric signal of the electric field treatment device 1 from the storage module 9110. The execution module 9111 is also configured to output a periodic direction switching drive signal to the direction control unit 916 based on the direction switching period of the alternating electric signal of the electric field treatment device 1 read from the storage module 910. The execution module 9111 is further configured to output a pulse signal to the inverter boost control unit 913 based on the electric field frequency and output AC voltage amplitude of the electric field treatment device and the reference voltage of the MCU control unit 911 all read from the storage module 9110. The pulse signal has a frequency same as the electric field frequency of the electric field treatment device 1 and an AC voltage amplitude same as the reference voltage of the MCU control unit 911. In this embodiment, the pulse signal output by the execution module 9111 to the inverter boost control unit 913 is a square wave signal with a frequency of 150 kHz, a voltage amplitude of 3.3V and a duty cycle of 50%.

The digital-to-analog conversion (DAC) module 9112 is electrically communicated with the DC power supply control unit 912 and has a DAC data register 91120. The digital-to-analog conversion (DAC) module 9112 can output a direct current with corresponding voltage to the DC power supply control unit 912 based on a value in the DAC data register 91120, so as to start the DC power supply control unit 912. The DAC data register 91120 of the digital-to-analog conversion module 9112 has a digital value of 212 corresponding to the MCU control unit's 911 reference voltage of 3.3V. The control module 9113 controls the execution module 9111 to perform the aforementioned corresponding functions. Based on the direction switching period of the alternating electric signal of the electric field treatment device 1 read by the execution module 9111, the control module 9113 controls connection and disconnection of communication between the digital-to-analog conversion module 9112 and the DC power supply control unit 912 and whether the execution module 9111 outputs the pulse signal to the inverter boost control unit 913 or not.

The DC power supply control unit 912 receives a direct current (DC) signal output by the digital-to-analog conversion module 9112 of the MCU control unit 911 and having a voltage approximately of 500 mV, and outputs a DC electric signal having a voltage approximately of 20V to the inverter boost control unit 913. The inverter boost control unit 913 has a boost module 9130 and an inverter module 9131 communicated with the boost module 9130. The boost module 9130 simultaneously receives a square wave signal output by the execution module 9111 of the MCU control unit 911 and having a frequency of 150 kHz, a voltage amplitude of 3.3V and a duty cycle of 50% and a DC electric signal output by the DC power supply control unit 912 had having a voltage of 20V. The boost module 9130 also performs superimposition processing and boost processing on the received square wave signal and the DC electric signal and outputs a square wave signal having a frequency of 150 kHz and an AC voltage amplitude of 80V to the inverter module 9131. The inverter module 9131 receives the square wave signal output by the boost module 9130 and having the frequency of 150 kHz and the voltage amplitude of 80V. The inverter module 9131 also performs inverter processing on the received square wave signal and outputs a square wave signal having a frequency of 150 kHz and a voltage amplitude of ±80V to the filter control unit 914. The filter control unit 914 performs filtering processing on the square wave signal received from the inverter module 9131 and having the frequency of 150 kHz and the voltage amplitude of ±80V to obtain a sine wave signal having a frequency of 150 kHz and an AC voltage peak value of 160V, and outputs a filtered sine wave signal with the frequency of 150 kHz and the AC voltage peak value of 160V to the AC voltage control unit 915. The AC voltage control unit 915 is simultaneously connected to the X-direction switch 917 and the Y-direction switch 918, and selectively applies the sine wave signal processed by the filter control unit 914 and having the frequency of 150 kHz and the AC voltage peak value of 160V to the two X-direction electrodes 22 or the two Y-direction electrodes 21 electrically connected to the AC voltage control unit 915 according to turn-on or turn-off of the X-direction switch 917 and the Y-direction switch 918, so as to generate the X-direction electric field 24 between the two X-direction electrodes 22 or generate the Y-direction electric field 23 between the two Y-direction electrodes 21 to perform tumor treatment on a malignant tumor site in an experimental animal or inhibit the proliferating cells in the tissue culture.

According to the periodic direction switching drive signal output by the executed module 9111 of the MCU control unit 911, the direction control unit 916 periodically controls the turn-on and turn-off of the X-direction switch 917 and the Y-direction switch 918. Specifically, the control module 9113 of the MCU control unit 911 controls the execution module 9111 to output a periodic direction switching drive signal to the direction control unit 916 according to the direction switching period of the electric field treatment device 1 that is read by the execution module 9111, and further alternately and periodically turns on the X-direction switch 917 and turns off the Y-direction switch 918 or turns off the X-direction switch 917 and turns on the Y-direction switch 918 via the direction control unit 916, so that the sine wave signal received by the AC voltage control unit 915 and having the frequency of 150 kHz and the AC voltage peak value of 160V is periodically and alternately applied to the two X-direction electrodes 22 and the two Y-direction electrodes 21 electrically connected to the AC voltage control unit 915, to periodically and alternately apply the X-direction electric field 24 and the Y-direction electric field 23 to the tumor site in the experimental animal or the proliferating cells in the tissue culture.

That is, when the MCU control unit 911 controls the direction control unit 916 to turn on the X-direction switch 917 and turn off the Y-direction switch 918, the AC voltage control unit 915 applies a sine-wave signal with the frequency of 150 kHz and the AC voltage peak value of 160V to the two X-direction electrodes 22 electrically connected thereto, and generates the X-direction electric field 24 between the two X-direction electrodes 22; and when the MCU control unit 911 controls the direction control unit 916 to turn off the X-direction switch 917 and turn on the Y-direction switch 918, the AC voltage control unit 915 applies a sine-wave signal with the frequency of 150 kHz and the AC voltage peak value of 160V to the two Y-direction electrodes 21 electrically connected thereto, and generates the Y-direction electric field 23 between the two Y-direction electrodes 21. In this embodiment, the periodic direction switching drive signal output from the execution module 9111 of the MCU control unit 911 to the direction control unit 916 has a duty cycle of 50% and a period of 2 Second. That is, the direction control unit 916 controls the X-direction switch 917 to be turned on at a first second, the Y-direction switch 918 to be turned on at a second second, the X-direction switch 917 to be turned on at a third second, the Y-direction switch to be turned on at a fourth second, and so on. The tumor electric field therapy system 1000 periodically and alternately applies an alternating electrical signal to the X-direction electrodes 22 and the Y-direction electrodes 21 by periodically turning on the X-direction switch 917 and the Y-direction switch 918, to treat the tumor site in the experimental animal or inhibit the proliferating cells in the tissue culture.

FIG. 2 is a waveform diagram of periodic direction switching drive signals for switching directions of electric fields generated between Y-direction electrodes 21 and X-direction electrodes 22, that is, a waveform diagram of drive signals of the direction control unit 916 for controlling the X-direction switch 917 and the Y-direction switch 918. The drive signals 31, 32 respectively correspond to the X-direction electrodes 22 and the Y-direction electrodes 21. The drive signals 31, 32 both have a duty cycle of 50%, a period of 2 seconds and two output states each having a high level of one and a low level of zero. The X-direction switch 917 and the Y-direction switch 918 are periodically and alternately turned on and turned off, and both have a continuous on-time of 1 second and a continuous off-time of 1 second. Only one of the X-direction switch 917 and the Y-direction switch 918 is turned on at the same time. That is, when the X-direction switch 917 is turned on, the X-direction electrodes 22 generate an X-direction AC electric field, and after the X-direction switch 917 is turned on for one second, the X-direction switch 917 is turned off, and the Y-direction switch 918 is turned on, and the Y-direction electrodes 21 generate a Y-direction AC electric field; after the Y-direction switch 918 is turned on for one second, the Y-direction switch 918 is turned off, and the X-direction switch 917 is turned on again; and such procedure is repeated periodically. The direction control unit 916 switches between the X-direction switch 917 and the Y-direction switch 918, so that the target region is alternately affected by the X-direction and Y-direction AC electric fields. The operating period and duty cycle of the drive signal can be adjusted and set randomly as needed.

As shown in FIG. 2, during a first time period T1, the X-direction switch 917 is turned on, the AC voltage control unit 915 applies the AC signal to the two X-direction electrodes 22 (also referred to as the first pair of insulated electrodes) to generate an X-direction electric field 24 (which can also be referred to as the first electric field) between the two X-direction electrodes 22 and having a field strength of at least 1V/cm, and meanwhile, the Y-direction switch 918 is turned off, and the Y-direction electric field 23 is turned off. At this time, the drive signal 31 is in an output state having the high level of one, and the drive signal 32 is in an output state having the low level of zero. During a second time period T2, the AC voltage control unit 915 applies the AC signal to the two Y-direction electrodes 21 (also referred to as the second pair of insulated electrodes) to generate a Y-direction electric field 23 (which can also be referred to as the second electric field) between the two Y-direction electrodes 21 and having a field strength of at least 1V/cm, and meanwhile the X-direction switch 917 is turned off, and the X-direction electric field 24 is turned off. At this time, the drive signal 31 is in an output state having the low level of zero, and the drive signal 32 is in an output state having the high level of one. That is, the X-direction electric field 24 is switched to the Y-direction electric field 23 after operating for the first time period T1, and the Y-direction electric field 23 is switched to the X-direction electric field 24 after operating for the second time period T2. By repeating such procedure, periodic switching between the X-direction electric field 24 generated between the two X-direction electrodes 22 and the Y-direction electric field 23 generated between the two Y-direction electrodes 21 can be implemented through the drive signals 31, 32 generated by the AC signal controller 920. The first time period T1 is the operating duration of the X-direction electric field 24 in each operating period, and is also the turn-off duration of the Y-direction electric field 23 in each operating period. The second time period T2 is the operating duration of the Y-direction electric field 23 in each operating period, and is also the turn-off duration of the X-direction electric field 24 in each operating period. In this embodiment, the first time period T1 and the second time period T2 are the same, and are both equal to half a period of the periodic switching drive signal 31, 32 generated by the AC signal controller 920. The first time period T1 and the second time period T2 are both 1 second. Optionally, the first time period of T1 and the second time period T2 can be both between 400 ms and 1000 ms.

The storage module 9110, the execution module 9111, the digital-to-analog conversion module 9112 and the control module 9113 of the MCU control unit 911, the DC power supply control unit 912, the inverter boost control unit 913, the filter control unit 914 and the AC voltage control unit 915 jointly form the AC signal generator 910 of the electric field treatment device 1. The storage module 9110, the execution module 9111, and the control module 9113 of the MCU control unit 911, the direction control unit 916, and the X-direction switch 917 and the Y-direction switch 918 electrically connected to the direction control unit 916 jointly form the AC signal controller 920. When the alternating electrical signal generated by the AC signal generator 910 is applied to the two X-direction electrodes 22, the X-direction electric field 24 is generated between the two X-direction electrodes 22. When the alternating electrical signal generated by the AC signal generator 910 is applied to the two Y-direction electrodes 21, the Y-direction electric field 23 is generated between the two Y-direction electrodes 21.

Before the X-direction electric field 24 between the two X-direction electrodes 22 and the Y-direction electric field 23 between the two Y-direction electrodes 21 need to be switched, the MCU control unit 911 disconnects a communication connection between the digital-to-analog conversion module 9112 and the DC power supply control unit 912 through the control module 9113, and controls the execution module 9111 to stop outputting the pulse signal to the inverter boost control unit 913 through the control module 9113, so as to prevent a therapeutic or inhibitory effect being affected when the X-direction electric field 24 generated between the two X-direction electrodes 22 and the Y-direction electric field 23 generated between the two Y-direction electrodes 21 are conducted simultaneously. After the execution module 9111 stops outputting the pulse signal to the inverter boost control unit 913 and the communication between the digital-to-analog conversion module 9112 and the DC power supply control unit 912 is disconnected, the direction control unit 916 is controlled to switch the X-direction switch 917 and the Y-direction switch 918.

After the MCU control unit 911 controls the direction control unit 916 to complete the switch between the X-direction electric field 24 generated between the two X-direction electrodes 22 and the Y-direction electric field 23 generated between the two Y-direction electrodes 21, the control module 9113 of the MCU control unit 911 controls the digital-to-analog conversion module 9112 to output a direct current signal with a voltage of 484 mV to the DC power supply control unit 912 to start the DC power supply control unit 912 and make the DC power supply control unit 912 to output a direct current signal with a voltage of 20V to the inverter boost control unit 913, and at the same time also controls the execution module 9111 to output a square-wave signal having a frequency of 150 kHz to the inverter boost control unit 913 and then the inverter boost control unit 913 outputs a sine wave having a frequency of 150 kHz formed by the square-wave signal and the direct current signal to the filter control unit 914, and the filter control unit 914 outputs a sine wave with a frequency of 150 kHz and an AC voltage peak value of 160V to the AC voltage control unit 915, so that the AC voltage control unit 915 can output the sine wave with a frequency of 150 kHz and an AC voltage peak value of 160V to the two X-direction electrodes 22 or the two Y-direction electrodes 21. The value in the DAC data register 91120 that corresponds to the direct current signal having a voltage of 484 mV and output by the digital-to-analog conversion module 9112 is 600 (484*4096/3300≈600).

The electric field treatment device 1 according to the present invention controls the DC power supply control unit 912 to output a direct current signal having a voltage increased or decreased as a constant speed through the control module 9113 of the MCU control unit 911 based on the direction switching period of the alternating electric signal obtained by the execution module 9111, so that the AC voltages of the alternating electrical signals applied to the two pairs of insulated electrodes 2 increase or decrease slowly at a constant speed during the respective operating periods, to prevent the X-direction switch 917 and the Y-direction switch 918 in the AC signal controller 920 from being impacted or damaged by a spike signal generated due to the sharp change of the AC voltage output by the AC voltage control unit 915 or to prevent a tingling sensation in experimental animals or humans that is caused by the sharp change of the AC voltage which generates a spike pulse that is transmitted to the X-direction electrodes 22 and the Y-direction electrodes 21 arranged around the tumor site when the MCU control unit 911 controls the direction control unit 916 to complete the switch between the X-direction electric field 24 and the Y-direction electric field 23. For details, refer to the following description.

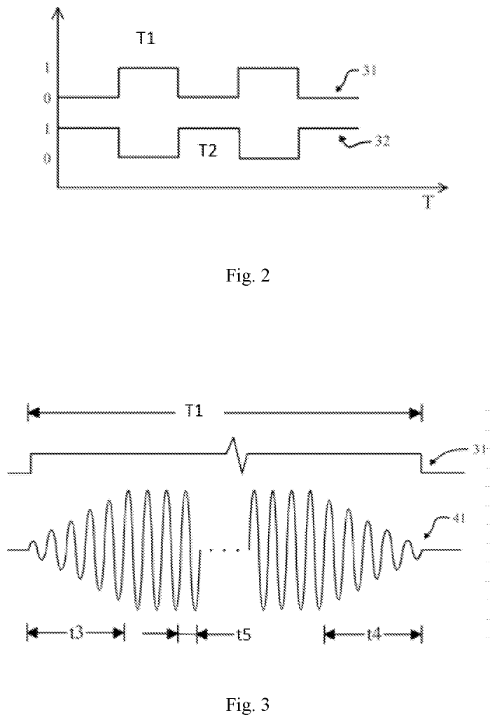

FIG. 3 is a schematic diagram of a periodic direction switching drive signal output by the MCU control unit 911 to the direction control unit 916 to generate an X-direction electric field 24 between two X-direction electrodes 22 for tumor-treating fields therapy. A partial waveform diagram of a drive signal 31 as the periodic direction switching drive signal is shown, and a signal 41 is a sine wave applied to the two X-direction electrodes 22. The first operating time period T1 is the continuous conduction on-time period of the X-direction electric field 24 in each period. A phase corresponding to the initial switching-on time period t3 of the alternating electric signal is a process in which the AC voltage applied to the two X-direction electrodes 22 increases from 0 to a specific value V or a process in which the AC voltage applied to the insulated electrode 2 reaches 90% of a peak to peak value of the voltage of the preset alternating electrical signal, and a phase corresponding to the final switching-off time period t4 of the alternating electric signal is a process in which the AC voltage applied to the two X-direction electrodes 22 decreases from a specific value V to 0. The specific value V is 90% of a peak value of the output AC voltage amplitude preset by the electric field treatment device 1. The continuous conduction on-time of the alternating electric signal in each period also has a plurality of intermediate on-time periods t5 between the initial switching-on time period t3 and the final switching-off time period t4 and the AC voltage of the alternating electrical signal output stably to the insulated electrodes 2 in the intermediate on-time periods t5 has a value between the specific value V and the output AC voltage peak value of the alternating electrical signal preset by the electric field treatment device 1. The initial switching-on time period t3 and the final switching-off time period t4 of the alternating electric signal are same.

As shown in FIG. 3, when the first time period T1 ends and is switched to the second time period T2, that is, when the X-direction electric field 24 is turned off and the Y-direction electric field 23 is turned on, the AC voltage of the alternating electric signal applied to the two X-direction electrodes 22 decreases to 0V, which can effectively prevent the AC signal generator 910 from simultaneously applying a voltage to both the X-direction electrodes 22 and the Y-direction electrodes 21 because switching is performed before the AC voltage of the alternating electric signal decreases to 0V when the AC signal applied to the X-direction electrodes 22 is discontinued, thereby avoiding a situation where the X-direction electric field 24 and the Y-direction electric field 23 exist at the same time and are overlapped. Herein, the initial switching-on time period t3 and the final switching-off time period t4 usually do not exceed 10% of the first time period T1 to avoid reducing an electric field intensity per unit time, to prolong time when the alternating electric signal applied to the X-direction electrodes 22 or the Y-direction electrodes 21 can generate an electric field intensity with a therapeutic effect to maximum extent. The sum of the initial switching-on time period t3, the final switching-off time period t4 and a plurality of intermediate on-time periods t5 is equal to the first time period T1. During the second time period T2, the X-direction electric field 24 generated between the X-direction electrodes 22 is turned off, and the Y-direction electric field 23 generated between the Y-direction electrodes 21 is turned on, thereby completing a periodic switching process. During the second time period T2, a sine wave applied to the two Y-direction electrodes 21 is the same as the signal 41. Details are not described herein again.

To eliminate the spike pulse, the MCU control unit 911 controls the change of the value output by the DAC data register 91120, so that the voltage of the direct current signal output by the DC power supply control unit 912 is slowly increased or slowly decreased at a constant speed, the AC voltage of the alternating electrical signal output to the AC voltage control unit 915 is slowly increased at a constant speed during the voltage step-up process and slowly decreased at a constant speed during the voltage step-down process, and the voltage change ΔV of the AC voltage control unit 915 per unit time is controlled within 5% of the specific voltage V output by the AC voltage control unit 915. The overall voltage increment is evenly divided in each unit time t. Assuming that it takes n unit times t to increase the AC voltage from 0 to the specific voltage V, the ΔV voltage change ΔV per unit time t is equal to the special voltage V divided by n and is within 5% of the specific voltage V, where n is a number of the unit time and not less than 20; and the initial switching-on time period t3 of the alternating electric signal is equal to multiply the number of the unit time by the unit time t, so that the initial switching-on time t3 divided by the unit time t is equal to the number of the unit time, and the initial switching-on time period t3 is not less than a value which is equal to multiply 20 by the unit time t. Similarly, the AC voltage output by the AC voltage control unit 915 is also decreased at a constant speed during the voltage step-down process. To eliminate the spike pulse, the overall voltage decrement is evenly divided in each unit time t, and assuming that it takes n unit times t to decrease the AC voltage from the specific voltage V to 0, the voltage change of the AC voltage control unit 915 per unit time t is equal to divide the specific voltage V by the number of the unit time and is within 5% of the specific voltage V, where n is not less than 20; and the final switching-off time period t4 of the alternating electric signal is equal to multiply the number of the unit time by the unit time t, that is, the number n is equal to divide the final switching-off time period t4 by the unit time t, and the final switching-off time period t4 is not less than a value that is equal to multiply 20 by the unit time t. That is, the AC voltage output by the AC voltage control unit 915 is increased to the specific voltage V at the constant speed or decreased from the specific voltage V to 0 at the constant speed during the preset time period. The AC voltage change per unit time t varies based on the different values of the specific voltage V.

As shown in FIG. 3, the voltage step-up processes and the voltage step-down processes for different specific voltages V are described in detail below.

A first embodiment of the voltage step-up process: the specific voltage V was 100V, the initial switching-on time period t3 of the alternating electric signal was 50 ms, the unit time t was preset to 1 ms, that is, the number n was 50, and therefore, the AC voltage change ΔV of the AC voltage control unit 915 per millisecond was calculated as the following formula: ΔV=V/n=100/50=2V, that is, the AC voltage output by the AC voltage control unit 915 needed to be increased by 2V every millisecond for 50 ms to increase the AC voltage applied to the insulated electrodes 2 by the AC voltage control unit 915 from 0V to 100V. A value storage in the DAC data register 91120 corresponding to the AC voltage of 100V was 375. A value variable ΔDAC of the DAC data register 91120 per unit time t was calculated as the following formula: ΔDAC=375/50≈8. The MCU control unit 911 increased the value of the DAC data register 91120 by 8 every millisecond. An output DC voltage value corresponding to the value of 4096 of the DAC data register 91120 was 3.3V. After calculation, an output DC voltage value corresponding to the value of 8 of the DAC data register 91120 was approximately equal to 6 mV which was calculated as (8*3.3/4096)*1000. That is, during the 50-ms period, the MCU control unit 911 increased the output DC voltage value of the digital-to-analog conversion module 9112 by about 6 mV every millisecond, and increased the output voltage of the DC power supply control unit 912 at a constant speed to control the AC voltage of the AC voltage control unit 915 to be increased by 2V every millisecond. After 50 constant changes or after 50 steps, the AC voltage applied to the Y-direction electrodes 21 by the AC voltage control unit 915 was increased from 0V to 100V. When the AC voltage applied to the Y-direction electrodes 21 by the AC voltage control unit 915 was increased to 100V, even if a 100% spike pulse occurred in a sharp voltage change, there was only a sharply changed voltage of 2V, 2V accounted for only 2% of 100V, and because the sharply changed voltage was still within an error range of 5% of the specific AC voltage, the sharply changed voltage could be ignored.

A second embodiment of the voltage step-up process: the specific voltage V was 120V, the initial switching-on time period t3 of the alternating electric signal was 50 ms, the unit time t was preset to 1 ms, that is, the number n was 50, and therefore, the AC voltage change ΔV of the AC voltage control unit 915 per millisecond was calculated as the following formula: ΔV=V/n=120/50=2.4V, that is, the AC voltage output by the AC voltage control unit 915 needed to be increased by 2.4V every millisecond for 50 ms to increase the AC voltage applied to the insulated electrodes 2 by the AC voltage control unit 915 from 0V to 120V. A value storage in the DAC data register 91120 corresponding to the AC voltage of 120V was 450. A value variable ΔDAC of the DAC data register 91120 per unit time t was calculated as the following formula: ΔDAC=450/50=9. The MCU control unit 911 increased the value of the DAC data register 91120 by 9 every millisecond. An output DC voltage value corresponding to the value of 4096 of the DAC data register 91120 was 3.3V. After calculation, an output DC voltage value corresponding to the value of 9 of the DAC data register 91120 was approximately equal to 7 mV which was calculated as (9*3.3/4096)*1000. That is, during 50-ms period, the MCU control unit 911 increased the output DC voltage value of the digital-to-analog conversion module 9112 by about 7 mV every millisecond, and increased the output voltage of the DC power supply control unit 912 at a constant speed to control the AC voltage of the AC voltage control unit 915 to be increased by 2.4V every millisecond. After 50 constant changes or 50 steps, the AC voltage applied to the Y-direction electrodes 21 by the AC voltage control unit 915 was increased from 0V to 120V. When the AC voltage applied to the Y-direction electrodes 21 by the AC voltage control unit 915 was increased to 120V, even if a 100% spike pulse occurred in a sharp voltage change, there was only a sharply changed voltage of 2.4V, 2.4V accounted for only 2% of 120V, and because the sharply changed voltage was still within an error range of 5% of the specific AC voltage, the sharply changed voltage could be ignored.

A third embodiment of the voltage step-up process: the specific voltage V was 160V, the initial switching-on time period t3 of the alternating electric signal was 50 ms, the unit time t was preset to 1 ms, that is, the number n was 50, and therefore, the AC voltage change ΔV of the AC voltage control unit 915 per millisecond met that ΔV=V/n=160/50=3.2V, that is, the AC voltage output by the AC voltage control unit 915 needed to be increased by 3.2V every millisecond for 50 ms to increase the AC voltage applied to the insulated electrodes 2 by the AC voltage control unit 915 from 0V to 160V. A value storage in the DAC data register 91120 corresponding to the AC voltage of 160V was 600. A value variable ΔDAC of the DAC data register 91120 per unit time t met that ΔDAC=600/50=12. The MCU control unit 911 increased the value of the DAC data register 91120 by 12 every millisecond. An output DC voltage value corresponding to the value of 4096 of the DAC data register 91120 was 3.3V. After calculation, an output DC voltage value corresponding to the value of 12 of the DAC data register 91120 was approximately equal to 10 mV which was calculated as (12*3.3/4096)*1000. That is, during 50-ms period, the MCU control unit 911 increased the output DC voltage value of the digital-to-analog conversion module 9112 by about 10 mV every millisecond, and increased the output voltage of the DC power supply control unit 912 at a constant speed to control the AC voltage of the AC voltage control unit 915 to be increased by 3.2V every millisecond. After 50 constant changes or 50 steps, the AC voltage applied to the Y-direction electrodes 21 by the AC voltage control unit 915 was increased from 0V to 160V. When the AC voltage applied to the Y-direction electrodes 21 by the AC voltage control unit 915 was increased to 160V, even if a 100% spike pulse occurred in a sharp voltage change, there was only a sharply changed voltage of 3.2V, 3.2V accounted for only 2% of 160 V, and because the sharply changed voltage was still within an error range of 5% of the specific AC voltage, the sharply changed voltage could be ignored.

In the aforementioned embodiments of the voltage step-up processes, values of the initial switching-on time periods t3 of the alternating electric signals were all preset to 50 ms. During the initial switching-on time period t3 of the alternating electric signal, an average voltage amplitude was low and did not reach the target voltage value. An intensity of the AC electric field generated was low, which had little impact on mitosis of cancer cells. The first operating time period T1 in each period was 1 second.

A first embodiment of the voltage step-down process: the specific voltage V was 100V, the final switching-off time period t4 of the alternating electric signal was 50 ms, the unit time t was preset to 1 ms, that is, the number n was 50, and therefore, the AC voltage change ΔV of the AC voltage control unit 915 per millisecond met that ΔV=V/n=100/50=2V, that is, the AC voltage output by the AC voltage control unit 915 needed to be decreased by 2V every millisecond for 50 ms to decrease the AC voltage applied to the insulated electrodes 2 by the AC voltage control unit 915 from 100V to 0V. A value in the DAC data register 91120 corresponding to the AC voltage of 100V was 375. A value variable ΔDAC of the DAC data register 91120 per unit time t was calculated as the following formula: ΔDAC=375/50≈8. The MCU control unit 911 reduced the value of the DAC data register 91120 by 8 every millisecond. An output value corresponding to the value of 4096 of the DAC data register 91120 was 3.3V. After calculation, the output DC voltage value corresponding to the value of 8 of the DAC data register 91120 was approximately equal to 6 mV which was calculated as (8*3.3/4096)*1000. That is, during 50-ms period, the MCU control unit 911 decreased the output value of the digital-to-analog conversion module 9112 by about 6 mV every millisecond, and decreased the output voltage of the DC power supply control unit 912 at a constant speed to control the AC voltage of the AC voltage control unit 915 to be decreased by 2V every millisecond. After 50 constant changes or after 50 steps, the AC voltage applied to the Y-direction electrodes 21 by the AC voltage control unit 915 was decreased from 100V to 0V. When the AC voltage applied to the Y-direction electrodes 21 by the AC voltage control unit 915 was decreased to 0V, even if a 100% spike pulse occurred in a sharp voltage change, there was only a sharply changed voltage of 2V, 2V accounted for only 2% of 100V, and because the sharply changed voltage was still within an error range of 5% of the specific AC voltage, the sharply changed voltage could be ignored.

A second embodiment of the voltage step-down process: the specific voltage V was 120V, the final switching-off time period t4 of the alternating electric signal was 50 ms, the unit time t was preset to 1 ms, that is, the number n was 50, and therefore, the AC voltage change ΔV of the AC voltage control unit 915 per millisecond met that ΔV=V/n=120/50=2.4V, that is, the AC voltage output by the AC voltage control unit 915 needed to be decreased by 2.4V every millisecond for 50 ms to decrease the AC voltage applied to the insulated electrodes 2 by the AC voltage control unit 915 from 120V to 0V. A value in the DAC data register 91120 corresponding to the AC voltage of 120V was 450. A value variable ΔDAC of the DAC data register 91120 per unit time t met that ΔDAC=450/50=9. The MCU control unit 911 reduced the value in the DAC data register 91120 by 9 every millisecond. An output DC voltage value corresponding to the value of 4096 of the DAC data register 91120 was 3.3V. After calculation, the output DC voltage value corresponding to the value of 9 of the DAC data register 91120 was approximately equal to 7 mV which was calculated as (9*3.3/4096)*1000. That is, during 50-ms period, the MCU control unit 911 decreased the output DC voltage value of the digital-to-analog conversion module 9112 by about 7 mV every millisecond, and decreased the output voltage of the DC power supply control unit 912 at a constant speed to control the AC voltage of the AC voltage control unit 915 to be decreased by 2.4V every millisecond. After 50 constant changes or after 50 steps, the AC voltage applied to the Y-direction electrodes 21 by the AC voltage control unit 915 was decreased from 120V to 0V. When the AC voltage applied to the Y-direction electrodes 21 by the AC voltage control unit 915 was decreased to 0V, even if a 100% spike pulse occurred in a sharp voltage change, there was only a sharply changed voltage of 2.4V, 2.4V accounted for only 2% of 120V, and because the sharply changed voltage was still within an error range of 5% of the specific AC voltage, the sharply changed voltage could be ignored.

TA third embodiment of the voltage step-down process: the specific voltage V was 160 V, the final switching-off time period t4 of the alternating electric signal was 50 ms, the unit time t was preset to 1 ms, that is, the number n was 50, and therefore, the voltage change ΔV of the AC voltage control unit 915 per millisecond met that ΔV=V/n=160/50=3.2V, that is, the AC voltage output by the AC voltage control unit 915 needed to be decreased by 3.2V every millisecond for 50 ms to decrease the AC voltage applied to the insulated electrodes 2 by the AC voltage control unit 915 from 160 V to 0V. A value in the DAC data register 91120 corresponding to the AC voltage of 160V was 600. A value variable ΔDAC of the DAC data register 91120 per unit time t met that ΔDAC=600/50=12. The MCU control unit 911 reduced the value in the DAC data register 91120 by 12 every millisecond. An output DC voltage value corresponding to the value of 4096 of the DAC data register 91120 was 3.3V. After calculation, the output DC voltage value corresponding to the value of 12 of the DAC data register 91120 was approximately equal to 10 mV which was calculated as (12*3.3/4096)*1000. That is, during 50-ms period, the MCU control unit 911 decreased the output value of the digital-to-analog conversion module 9112 by about 10 mV every millisecond, and decreased the output DC voltage of the DC power supply control unit 912 at a constant speed to control the AC voltage of the AC voltage control unit 915 to be decreased by 3.2V every millisecond. After 50 constant changes, the AC voltage applied to the Y-direction electrodes 21 by the AC voltage control unit 915 was decreased from 160 V to 0V. When the AC voltage applied to the Y-direction electrodes 21 by the AC voltage control unit 915 was decreased to 0V, even if a 100% spike pulse occurred in a sharp voltage change, there was only a sharply changed voltage of 3.2V, 3.2V accounted for only 2% of 160 V, and because the sharply changed voltage was still within an error range of 5% of the specific AC voltage, the sharply changed voltage could be ignored.

In the aforementioned embodiments of the voltage step-down processes, values of the final switching-off time periods t4 of the alternating electric signals were all preset to 50 ms. Because an average voltage amplitude was low and did not reach the target voltage value during the final switching-off time period t4 of the alternating electric signal, an intensity of the AC electric field generated was low, which had little impact on mitosis of cancer cells. The first operating time T1 of the electric field in each period was 1 second.

The control module 9113 of the MCU control unit 911 of the electric field treatment device 1 of the tumor electric field therapy system 1000 according to the present disclosure controls the value change of the DAC data register 91120 per unit time to make the voltage of the DC electric signal output by the DC power supply control unit 912 to be slowly increased at a constant speed during the voltage step-up process and slowly decreased at a constant speed during the voltage step-down process. In this way, the AC voltages of the alternating electrical signals applied by the AC voltage control unit 915 to the Y-direction electrodes 21 or the X-direction electrodes 22 increase at a constant speed during the initial switching-on time period and decrease at a constant speed during the final switching-off time period in respective periodic operating periods, and the AC voltage change per unit time is controlled to be within 5% of the operating AC voltage of the alternating electrical signals output by the AC voltage control unit 915 during the intermediate on-time period. Therefore, not only damage of the X-direction switch 917 or the Y-direction switch 918 of the AC signal controller 920 that is caused by a sharp change in the AC voltage applied to the Y-direction electrodes 21 and the X-direction electrodes 22 during direction switching can be prevented, but also a tingling sensation in experimental animals or humans that is caused by the Y-direction electrodes 21 and the X-direction electrodes 22 arranged on the body surface corresponding to the tumor site due to the sharp change in the AC voltage is avoided.

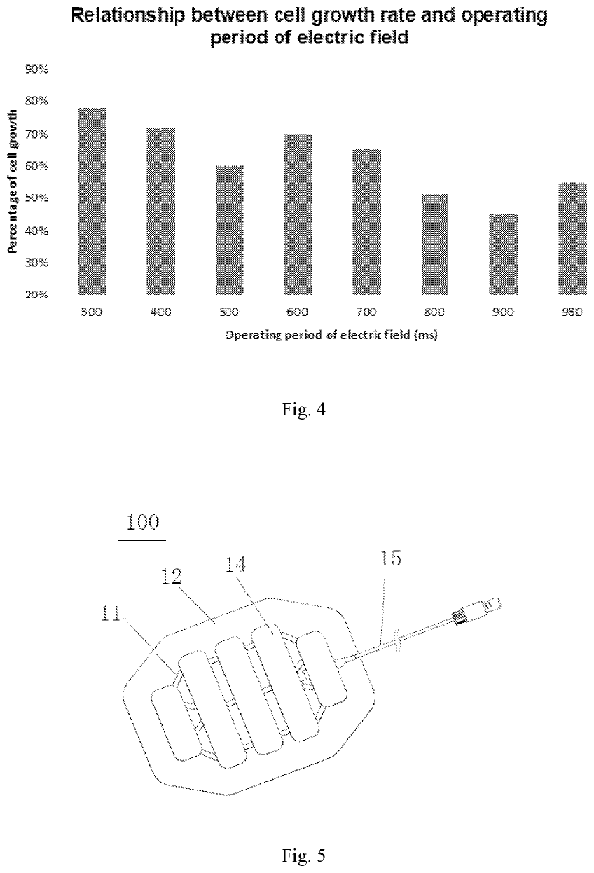

FIG. 4 shows effects of applying electric fields with different operating periods on cell proliferation during glioma cell culture. Switching rates of the applied electric fields in different directions are different. The inhibitory effects of the tumor treatment electric fields on proliferating cells in the tissue culture and the malignant cells in the experimental animals are different.

Experiments have revealed that when glioma cells are cultured in a culture dish and two pairs of mutually perpendicular 150 kHz AC signals are applied around the glioma cells, the proliferation of the cells is affected by changing the switching rates of the X-direction electric field 24 and the Y-direction electric field 23. As shown in FIG. 2, the X-direction electric field 24 is switched to the Y-direction electric field 23 after operating for the first time period T1, and the Y-direction electric field 23 is switched to the X-direction electric field 24 after operating for the second time period T2. Such procedure is repeated, and the first time period T1 and the second time period T2 are the same. Experimental results show that when the first time period T1 and the second time period T2 are between 400 ms and 980 ms, inhibitory effects on cell proliferation are better than those at other rates. Preferably, when the first time period T1 and the second time period T2 are both about 500 ms and are both between 700 ms and 980 ms, the inhibitory effect on cell proliferation is better. In this embodiment, U87MG glioma is used for cell tissue culture. However, to obtain an inhibitory result of the switching rate on cell proliferation, not only this cell can be used, but also other rapidly proliferating cells can also be used.

The two Y-direction electrodes 21 and the two X-direction electrodes 22 in the tumor electric field therapy system 1000 have the same structure. There may be different embodiments of the insulated electrode in the present disclosure. For the insulated electrode in the present disclosure, the following multiple embodiments are provided:

### A First Embodiment of the Insulated Electrode

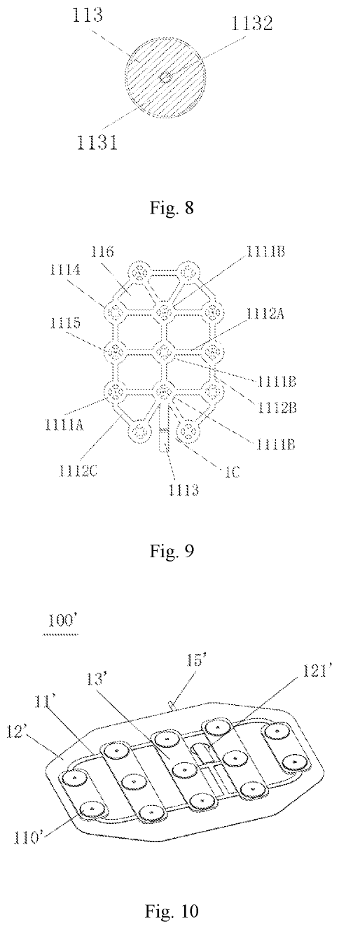

FIG. 5 to FIG. 9 show an insulated electrode 100 according to the first embodiment of the present disclosure. The insulated electrode 100 can be applied to a body surface corresponding to a tumor site in a torso of a patient to perform electric field treatment on the tumor site, and includes a flexible adhesive-backed layer 12, an electrical functional component 11 adhered to the adhesive-backed layer 12, a plurality of supporting members 13 adhered to the adhesive-backed layer 12, a plurality of adhesive members 14 respectively adhered to the corresponding supporting members 13, and a wire 15 electrically connected to the electrical functional component 11. The insulated electrode 100 in the present disclosure is attached to the body surface corresponding to the tumor site of the patient through the adhesive-backed layer 12, and applies an alternating electric field to the tumor site of the patient through the electrical functional component 11 to interfere with or prevent mitosis of tumor cells of the patient, thereby achieving a tumor treatment purpose.

Referring to FIG. 7, the electrical functional component 11 includes a flexible circuit board 111, multiple insulating plates 112 and multiple dielectric elements 113 respectively provided on two opposite sides of the flexible circuit board 111, and multiple temperature sensors 114 soldered on the flexible circuit board 111. The temperature sensors 114 and the dielectric elements 113 are located on the same side of the flexible circuit board 111. A plurality of the dielectric elements 113 are provided on one side of the flexible circuit board 111 that is closer to a body surface of a patient, and a plurality of the insulating plates 112 are provided on the other side of the flexible circuit board 111 that is away from the body surface of the patient. The electrical functional component 11 is tightly adhered to the adhesive-backed layer 12 through respectively affixing the insulating plates 112 and corresponding parts of the flexible circuit board 111 to the adhesive-backed layer 12. The insulated electrode 100 applies the alternating electric signal generated by the electric field generator (not shown in the figure) onto the tumor site of the patient through the plurality of the dielectric elements 113 provided on the flexible circuit board 111, thereby performing electric field treatment on the tumor site of the patient.

The flexible circuit board 111 includes multiple main bodies 1111 arranged in an array, multiple connecting portions 1112 each located between adjacent main bodies 1111, and a wiring portion 1113 electrically connected to the wire 15. The wiring portion 1113 may be arranged by laterally extending one connecting portion 1112, or may be arranged by laterally extending one end from the main body 1111. The plurality of the dielectric elements 113 and the plurality of the main bodies 1111 are respectively arranged in one-to-one correspondence. Each dielectric element 113 is welded to the corresponding main body 1111. Each main body 1111 is provided at the end of the corresponding connecting portion 1112. Each main body 1111 extends from the end of the corresponding connecting portion 1112 and is at least connected to two main bodies 1111 adjacent thereto through the corresponding connecting portions 1112. Each main body 1111 is arranged substantially in a shape of a circular sheet. Optionally, the main body 1111 can also be strip-shaped or belt-shaped and can be integrally formed with the connecting portion 1112.

The number of the main bodies 1111 is at least 10, and the number of the dielectric elements 113 is also at least 10. The dielectric elements 113 have the same distribution as the main bodies 1111, and are distributed in an array region with at least four rows and three columns, which can increase a coverage area of the insulated electrode 100, enhance the intensity of the electric field applied to the tumor site for tumor-treating fields therapy, and increase a range of covering the tumor site by the alternating electric field, thereby improving a therapeutic effect. The main bodies 1111 are arranged at intervals, to form a plurality of open spaces 116 between multiple adjacent main bodies 1111 to allow skin of the body surface corresponding to the tumor site of the patient that is covered by the insulated electrode 100 to breathe freely after the insulated electrode 100 is applied to the body surface corresponding to the tumor site of the patient. The wiring portion 1113 is partially located between the multiple main bodies 1111 and is partially arranged in the open space 116 surrounded by the multiple main bodies 1111, which can avoid an increase in manufacturing costs due to an excessively large overall size of the flexible circuit board 111.

The main body 1111 has a circular sheet-shaped structure and a diameter of 21 mm to 22 mm. A distance between the main bodies 1111 in adjacent rows is at least 1 mm, and a distance between the main bodies 1111 in adjacent columns is at least 1 mm. That is, a distance between two main bodies 1111 which are located in the same row and adjacent columns is at least 1 mm, and the distance between two adjacent main bodies 1111 in the same column is also at least 1 mm. A distance between two main bodies 1111 which are located in the same row and spaced columns is at least 23 mm, and a distance between two main bodies 1111 which are located in the same column and spaced rows is also at least 23 mm.

Preferably, the numbers of the main bodies 1111 and the dielectric elements 113 are both 13, and the main bodies 1111 and dielectric elements 113 can be both distributed in an array region with five rows and three columns, or an array region with five rows and five columns. From the perspective of row arrangement, each of the first row and the last row is provided with two main bodies 1111, and each of the three middle rows is provided with three main bodies 1111. In this embodiment, the main bodies 1111 are distributed in an array region with five rows and five columns. From the perspective of column arrangement, the first, third and fifth columns are each provided with three main bodies 1111, and the second and fourth columns are each provided with two main bodies 1111. Specifically, the two main bodies 1111 in the first row are located in the second and fourth columns respectively, the three main bodies 1111 in each of the three middle rows are located in the first, third and fifth columns respectively, and the two main bodies 1111 in the last row are respectively located in the second row and the fourth row. Two adjacent main bodies 1111 in each row are arranged in spaced columns, and the main bodies 1111 in each of the second and fourth columns are arranged in spaced rows. Two adjacent main bodies 1111 in each of the first, third, and fifth columns are arranged in adjacent rows. The distances between a plurality of two adjacent main bodies 1111 in the same row are equal. The distances between a plurality of two adjacent main bodies 1111 in the same column are equal. The two main bodies 1111 in the last row are arranged in a disconnected manner to form a gap 1C located therebetween. The wiring portion 1113 is laterally extended by the main body 1111 located in the fourth row and third column. The wiring portion 1113 passes through the gap 1C formed between the two main bodies 1111 in the last row.

All the main bodies 1111 of the flexible circuit board 111 are spaced and distributed within a region with a minimum length of 109 mm and a minimum width of 109 mm. The minimum size of an area where the main bodies 1111 are located is about 109 mm×109 mm. A diameter of the main body 1111 is 21 mm. The distances between two adjacent main bodies 1111 in each row are the same and are at least 23 mm. The distances between two adjacent main bodies 1111 in each of the second and fourth columns are the same and are at least 67 mm. The distances between two adjacent main bodies 1111 in each of the first, third, and fifth columns are the same and are at least 1 mm. The aforementioned insulated electrode 100 with the smallest size for distributing the main bodies 1111 is suitable for use by children with smaller bodies.

In order to prevent the therapeutic effect from being affected due to overlapping of the electrical functional components 11 after the insulated electrode 100 is adhered to the body surface of the patient corresponding to the tumor site, the area for distributing the main bodies 1111 has the maximum length of 219 mm and the maximum width of 163 mm. That is, all the main bodies 1111 of the flexible circuit board 111 are spaced and distributed within a maximum region of 219 mm×163 mm. A diameter of the main body 1111 is 22 mm. The maximum distance between the main bodies 1111 of the flexible circuit board 111 in adjacent rows is about 28 mm, and the maximum distance between the main bodies 1111 of the flexible circuit board 111 in adjacent columns is about 50 mm. That is, the maximum distance between two main bodies 1111 located in the same column and adjacent rows is about 28 mm, and the maximum distance between two main bodies 1111 located in the same row and adjacent columns is 50 mm. The maximum size of the aforementioned flexible circuit board 111 is suitable for most adult patients. When a waist of the patient has a large size, two pairs of insulated electrodes 100 can be adhered transversely to the body surface corresponding to the waist of the patient. When a waist of the patient has a small size, two pairs of insulated electrodes 100 can be longitudinally adhered to the body surface corresponding to the waist of the patient. When the waist of the patient has an intermediate size, one pair of insulated electrodes 100 can be adhered transversely to the body surface corresponding to the waist of the patient, another pair of insulated electrodes 100 can be adhered longitudinally to the body surface corresponding to the waist of the patient, and the two pairs of insulated electrodes 100 are adhered to the body surface around the waist of the patient.

Each connecting portion 1112 connects two adjacent main bodies 1111, and each conductive pad 1114 is provided on the corresponding main body 1111 located at the end of the connecting portion 1112. The connecting portions 1112 include a plurality of first connecting portions 1112A each connecting with two adjacent main bodies 1111 located in the same row and spaced columns, a plurality of second connecting portions 1112B each connecting with two main bodies 1111 located in the same column and adjacent rows, and a plurality of third connecting portions 1112C each connecting with the two main bodies 1111 arranged diagonally in adjacent rows and adjacent columns. The first connecting portions 1112A are arranged between a plurality of two adjacent main bodies 1111 located in each row and spaced columns and have the same length. A length of the first connecting portion 1112A is approximately 23 mm to 50 mm. The second connecting portions 1112B are arranged between a plurality of two adjacent main bodies 1111 located in each of the first, third and fifth columns and have the same length. A length of the second connecting portion 1112A is approximately 1 mm to 28 mm. A length of the third connecting portion 1112C is greater than half the length of the first connecting portion 1112A. The length of the third connecting portion 1112C is greater than the length of the second connecting portion 1112B. The first connecting portion 1112A and the second connecting portion 1112B are both arranged substantially in a shape of “−”. The third connecting portion 1112C is generally arranged in a shape of “L” or a shape of inclined “−”. There are 8 third connecting portions 1112C, namely, a third connecting portion 1112C located between the main body 1111 in the first row and second column and the main body 1111 in the first column and second row, a third connecting portion 1112C located between the main body 1111 in the first row and second column and the main body 1111 in the second row and third column, a third connecting portion 1112C located between the main body 1111 in the second row and third column and the main body 1111 in the first row and fourth column, a third connecting portion 1112C located between the main body 1111 in the first row and fourth column and the main body 1111 in the second row and fifth column, a third connecting portion 1112C located between the main body 1111 in the last row and the second column and the main body 1111 in the first column and fourth row, a third connecting portion 1112C located between the main body 1111 in the last row and second column and the main body 1111 in the fourth row and third column, a third connecting portion 1112C located between the main body 1111 in the fourth row and third column and the main body 1111 in the last row and fourth column, and a third connecting portion 1112C located between the main body 1111 in the last row and fourth column and the main body 1111 in the fourth row and fifth column. Preferably, the length of the first connecting portion 1112A is greater than the diameter of the main body 1111. The length of the second connecting portion 1112B is less than the diameter of the main body 1111. The first connecting portion 1112A and the second connecting portion 1112B are arranged perpendicularly. The third connecting portion 1112C and the adjacent first connecting portion 1112A are arranged to form an acute angle. The second connecting portion 1112B and the adjacent third connecting portion 1112C are also arranged to form an acute angle.

According to the distribution positions of the main bodies 1111 in the array, the main bodies 1111 can be divided into a plurality of peripheral main bodies 1111A located on the periphery of the array and at least one central main body 1111B surrounded by the peripheral main bodies 1111A and located in the core of the array. Specifically, there are 10 peripheral main bodies 1111A and 3 central main bodies 1111B in the same column. Each peripheral main body 1111A is connected to the corresponding central main body 1111B through the corresponding connecting portion 1112. Two adjacent peripheral main bodies 1111A are electrically connected with each other through the corresponding first connecting portion 1112A, or the corresponding second connecting portion 1112B, or the corresponding third connecting portion 1112C. Specifically, two adjacent peripheral main bodies 1111A in the same column are connected with each other through the second connecting portion 1112B, two adjacent peripheral main bodies 1111A in the same row are connected with each other through the first connecting portion 1112A, and two adjacent peripheral main bodies 1111A arranged diagonally in adjacent rows and adjacent columns are connected with each other through the third connecting portion 1112C. The peripheral main bodies 1111A and the first connecting portions 1112A each located between the corresponding two adjacent peripheral main bodies 1111A, the second connecting portions 1112B each located between the corresponding two adjacent peripheral main bodies 1111A, and the third connecting portions 1112C each located between the corresponding two adjacent peripheral main bodies 1111A together form an octagonal shape with one end open. The peripheral main bodies 1111A are arranged axially symmetrically, and has a symmetry axis overlapping with a straight line on which the 3 central main bodies 1111B are located.

The central main bodies 1111B are 3 main bodies 1111 in the third column. Each central main body 1111B and an adjacent peripheral main body 1111A are connected with each other through the corresponding first connecting portion 1112A or the corresponding third connecting portion 1112C. Two adjacent central main bodies 1111B are electrically connected with each other through the corresponding second connecting portion 1112B. Specifically, the central main body 1111B and the adjacent peripheral main body 1111A in the same row are electrically connected with each other through the corresponding first connecting portion 1112A. The central main body 1111B and the peripheral main body 1111A arranged diagonally in adjacent rows and adjacent columns are electrically connected with each other through the corresponding third connecting portion 1112C, so that each central main body 1111B can be connected with at least two adjacent peripheral main bodies 1111A through the corresponding connecting portions 1112, to ensure that of the peripheral main bodies 1111A and the central main bodies 1111B have relatively fixed positions and stable connections, thereby facilitating welding the dielectric elements 113 to the flexible circuit board 111. That is, the central main body 1111B in the third row and the peripheral main body 1111A in the same row are connected with each other only through the corresponding first connecting portion 1112A, and the central main body 1111B in the third row and the adjacent peripheral main bodies 1111A arranged diagonally in adjacent rows and adjacent columns are disconnected with each other. Each of the other two central main bodies 1111B is not only connected with the peripheral main bodies 1111A arranged diagonally in adjacent rows and adjacent columns through the corresponding third connecting portion 1112C, but also is connected with the peripheral main body 1111A in the same row through the corresponding first connecting portion 1112A.

Preferably, as shown in FIG. 9, all the main bodies 1111 of the flexible circuit board 111 are spaced and distributed within a region of 165 mm×109 mm. The diameter of the main body 1111 is 21 mm. The distance between the main bodies 1111 in the adjacent rows is 15 mm, and the distance between the main bodies 1111 in the same row and spaced columns is 23 mm. That is, the distance between two main bodies hull located in the same column and adjacent rows is about 15 mm, and the distance between two main bodies 1111 located in the same row and spaced columns is 23 mm. That is, the length of the second connecting portion 1112B is approximately 15 mm, and the length of the first connecting portion 1112A is approximately 23 mm. A width of the wiring portion 113 is 8 mm. The widths of the first connecting portion 1112A, the second connecting portion 1112B and the third connecting portion 1112C are all 4.5 mm. Areas of the open spaces 116 of the flexible circuit board 111 that are formed between the multiple main bodies 1111 are not completely the same. In this embodiment, in all the open spaces 116, the open spaces 116 each jointly formed by 4 main bodies 1111 among the 9 main bodies 1111 in the middle three rows and two of which are in the same row and spaced columns and two of which are in the same column and adjacent rows has the largest area, namely, approximately 1065 mm². An area of the open space 116 formed by each three main bodies 1111 in the first row and adjacent rows thereof or in the last row and adjacent rows thereof is the smallest. Specifically, the open space 116 formed by two main bodies 1111 located in the same row and spaced columns and one main body 1111 arranged diagonally in a spaced row and a spaced column relative to the two main bodies 1111 has the smallest area, namely, approximately 470 mm².

The wiring portion 1113 is partially located between the multiple main bodies 1111 and is partially arranged in the open space 116 surrounded by the multiple main bodies 1111, which can avoid an increase in manufacturing costs due to an excessively large overall size of the main bodies 1111. The wiring portion 1113 is arranged by laterally extending one of the two main bodies 1111 located at two opposite ends of the 3 main bodies 1111 in the third column. Specifically, the wiring portion 1113 is arranged by laterally extending the main body 1111 located in the fourth row and third column. The wiring portion 1113 is arranged by extending the central main body 1111B at the end away from a region where the array of the main body 1111 is located. The wiring portion 1113 is located between two third connecting portions 1112C and is connected to the central main body 1111B connected with the two third connecting portions 1112C at the same time and located at the end. The wiring portion 1113 and the two third connecting portions 1112C that are together connected to the same central main body 1111B with the wiring portion 1113 are generally arranged in an arrow shape. The wiring portion 1113 extends between two peripheral main bodies 1111A in the same row that are arranged in a disconnected manner. The wiring portion 1113 and the first connecting portion 1112A are arranged substantially perpendicularly. The wiring portion 1113 and the second connecting portion 1112B are arranged substantially in parallel. The wiring portion 1113 is arranged substantially in a shape of “−”. A width of the wiring portion 1113 is at least 4 mm. Preferably, the width of the wiring portion 1113 is 4 mm to 8 mm. The included angle between the wiring portion 1113 and the third connecting portion 1112C that are both connected to the same main body 1111 is an acute angle. In other embodiments, the wiring portion 1113 can also be arranged by laterally extending the main body 1111 or the central main body 1111B in the second row and third column; and the two main bodies 1111 in the first row are disconnected. The wiring portion 1113 passes through the gap formed between the two main bodies 1111. In other embodiments, the wiring portion 1113 can also be arranged by laterally extending one second connecting portion 1112B located between two adjacent central main bodies 1111B, and the wiring portion 1113 is perpendicular to the second connecting portion 1112B. The wiring portion 1113 and the second connecting portion 1112B extended to form the wiring portion 1113 are generally arranged into a shape of “T”.

Each conductive pad 1114 is provided on the side of the corresponding main body 1111 facing toward the corresponding dielectric element 113 for being welded to the corresponding dielectric element 113 via soldering tin (not shown in the figure) to mount the corresponding dielectric element 113 onto the corresponding main body 1111 of the flexible circuit board 111. The center of the conductive pad 1114 is overlapped with the center of the main body 1111. Each conductive pad 1114 has 4 conductive cores 1115 protruding or exposed from the main body 1111. The conductive cores 1115 are arranged centrally symmetrically, which can effectively prevent the position of the dielectric element 113 from being shifted due to accumulation of soldering tin (not shown in the figure) during the welding process. The 4 conductive cores 1115 are arranged at intervals, which can reduce the amount of copper foil used to manufacture the conductive cores 1115 and reduce material costs. In addition, this can also reduce the amount of soldering tin (not shown in the figure) used to weld the conductive cores 1115 to the dielectric element 113, thereby further reducing the material costs.

The 4 conductive cores 1115 of the same conductive pad 1114 all have petal-shaped structures. Each conductive core 1115 includes an inner arc (not numbered) and an outer arc (not numbered) connected end to end. The inner arc (not numbered) and the outer arc (not numbered) of the conductive core 1115 are arranged axially symmetrically. The inner arcs (not numbered) of the 4 conductive cores 1115 of the same conductive pad 1114 are all recessed toward the center of the conductive pad 1114. The outer arcs (not numbered) of the 4 conductive cores 1115 of the same conductive pad 1114 all protrude in a direction of leaving the center of the conductive pad 1114. The 4 conductive cores 1115 constituting the conductive pad 1114 are both arranged centrally symmetrically and axially symmetrically, and each conductive core 1115 is also axially symmetrically. Therefore, when the 4 conductive cores 1115 of the conductive pad 1114 of the main body 1111 are welded to the dielectric element 113, stress balance between welding points is ensured, overall welding balance of the dielectric element 113 is ensured, and welding quality is improved, which prevents a welding joint on the side of the dielectric element 113 at a larger distance from the main body part 1111 being weak and prone to break because the dielectric element 113 is tilted when the welding stress is unbalanced, and can also avoid affecting the adhesion of the insulated electrode 100. The outer arcs (not numbered) of the 4 conductive cores 1115 of the same conductive pad 1114 are generally located on the same circumference.

Each insulating plate 112 is arranged substantially in a shape of a circular sheet. Each insulating plate 112 is made of an insulated material and is adhered to the side of the main body 1111 of the flexible circuit board 111 that is away from the body surface of the patient through a sealant (not shown in the figure), which can also provide a flat welding plane for a welding operation between the conductive pad 1114 and the dielectric element 113 while enhancing the strength of the flexible circuit board 111, thereby improving product yield. The insulating plates 112 can prevent water vapor in the air on the side of the electrical functional component 11 that is away from the body surface of the patient from entering the electrical functional component 11, thereby preventing an electrical connection between the main bodies 1111 and the dielectric elements 113 being affected because the water vapor comes into contact with the soldering tin (not shown in the figure) between the dielectric elements 113 and the main bodies 1111. The insulating plates 112 are arranged in a one-to-one correspondence with the main bodies 1111, and have the same arrangement as the main bodies 1111.

Each dielectric element 113 is arranged in a shape of a circular sheet. Each dielectric element 113 is made of a material with a high dielectric constant, which has a property of blocking the direct current and conducting the alternating current, thereby ensuring safety of human body. Each dielectric element 113 has a dielectric constant that is at least greater than 1000. A ring-shaped metal layer 1131 is attached to the side of the dielectric element 113 facing toward the main body 1111, which can be welded to the conductive pad 1114 on the main body 1111 via soldering tin (not shown in the figure). The gap (not shown in the figure) formed between the dielectric element 113 and the main body 1111 is filled with the sealant (not shown in the figure), so as to protect the soldering tin (not shown in the figure) between the dielectric element 113 and the main body 1111, avoid the alternating electric field is unable to be applied to the tumor site of the patient through the dielectric element 113 because the welding joint between the dielectric element 13 and the main body 1111 is broken when the dielectric element 113 is affected by external force, and further avoid affecting the electrical connection between the dielectric element 113 and the main body 1111 because the water vapor in the air enters the gap (not shown in the figure) and corrodes the soldering tin (not shown in the figure) between the dielectric element 113 and the main body 1111.

The outer ring of the metal layer 1131 is spaced apart from the outer edge of the corresponding dielectric element 113, so as to prevent the soldering tin (not shown in the figure) between the metal layer 1131 of the dielectric element 113 and the main body 1111 from melting and overflowing to the main body 1111 when heated and prevent the direct current which is unimpeded by the dielectric element 113 from directly acting on the body surface of the patient when the insulated electrode 100 is adhered to the body surface of the patient corresponding to the tumor site. The dielectric element 113 has a through-hole 1132 for accommodating the temperature sensor 114. The edge of the through-hole 1132 is spaced apart from the inner ring of the corresponding metal layer 1131, so as to avoid a short circuit of the temperature sensor 114 that is caused because the soldering tin (not shown in the figure) between the metal layer 1131 of the dielectric element 113 and the main body 1111 is melted and spreads toward the through-hole 1132 when heated. The main body 1111, the insulating plate 112 and the dielectric element 113 are arranged in one-to-one correspondence, and have centers on the same straight line. The insulating plates 112 and the dielectric elements 113 have the same arrangement as the main bodies 1111, and are both distributed in an array region with five rows and five columns.

Each main body 1111 of the flexible circuit board 111, each insulating plate 112 located on one side of the corresponding main body 1111 away from the patient's body surface and each dielectric element 113 located on the other side of the corresponding main body 1111 facing toward the patient's body surface jointly form the electrode element 110 of the electrically functional component 11. The electrode elements 110 of the electrical functional component 11 have the same arrangement as the main bodies 1111 of the flexible circuit board 111. Each connecting portion 1112 is located between two adjacent electrode elements 110.

Each temperature sensor 114 is fixed on the corresponding main body 1111 and configured to monitor temperature of the corresponding adhesive member 14, thereby monitoring the temperature of the human skin attached with the corresponding adhesive member 14. When one temperature monitored by the temperature sensors 114 exceeds an upper limit of human body's safety temperature threshold, the electric field generator (not shown in the figure) can promptly reduce or cut off the alternating current transmitted to the insulated electrode 100 to avoid a low-temperature burn to the human body. Each temperature sensor 114 is welded to the corresponding main body 1111 and then sealed with the sealant (not shown in the figure) to avoid being corroded and failure by water vapor. The temperature sensors 114 are provided on the peripheral main bodies 1111 among the plurality of main bodies 1111 arranged in an array. That is, each temperature sensor 114 is provided on the corresponding peripheral main body 1111A.

The wire 15 has one end welded to the wiring portion 1113 of the electrical functional component 11, and the other end provided with a plug (not numbered) that is electrically connected to the electric field generator (not shown in the figure). The plug (not numbered) of the wire 15 can be directly plugged into the electric field generator (not shown in the figure), or can be plugged into the adapter (not shown in the figure) of the tumor electric field therapy system, and then is electrically connected to the electric field generator (not shown in the figure) through the adapter (not shown in the figure) to achieve the electrical connection between the wire 15 and the electric field generator (not shown in the figure). A heat shrink sleeve 151 covers a joint between the wire 15 and the wiring portion 1113 of the flexible circuit board 111 for sealing, insulating, protecting and increasing the strength and support of the joint between the wire 15 and the wiring portion 1113 of the flexible circuit board 111, thereby avoiding breakage of the joint between the wire 15 and the electrical functional component 11 and achieving dust-proof and waterproof purposes.

Each supporting member 13 is provided in a shape of a sheet. Each supporting member 13 is adhered to the adhesive-backed layer 12 by surrounding the electrode elements 110 arranged in rows. The plurality of supporting members 13 are arranged at intervals. Each supporting member 13 has a plurality of spaced through-holes 131 corresponding to the electrode elements 110. A thickness of each supporting member 13 is basically consistent with a thickness of the corresponding electrode element 110. A plane where a top of each supporting members 13 is located is at the same vertical height as the surface on the side of the corresponding electrode element 110 facing toward the body surface of the patient, that is, the surface on the side of each supporting member 13 closer to the body surface of the patient is flush with the surface on the side of the corresponding dielectric element 113 closer to the body surface of the patient, so that the adhesive members 14 can flatly cover the supporting members 13 and the electrode elements 110, thereby improving comfort of applying the insulated electrode 100. The supporting members 13 can be made of an insulated material that is soft, chemically stable, light in weight, less easily deformed, and non-toxic and that is formed by integrating polyethylene (PE) material or PET material or thermally conductive silicone sheet or polyurethane, polyethylene, dispersant, a flame retardant, carbon fiber, and the like. Preferably, the supporting members 13 are flexible foams.

Each adhesive member 14 is arranged in a shape of a sheet and has one side attached to the corresponding supporting member 13 and the corresponding dielectric element 113 and the other side attached to the body surface of the patient. Each adhesive member 14 is a conductive hydrogel and can be used as a conductive medium to conduct the alternating current passing through the corresponding dielectric element 113 to the tumor site of the patient. The number of adhesive members 14 is the same as the number of supporting members 13. The size of each adhesive member 14 is substantially the same as the size of the corresponding supporting member 13.

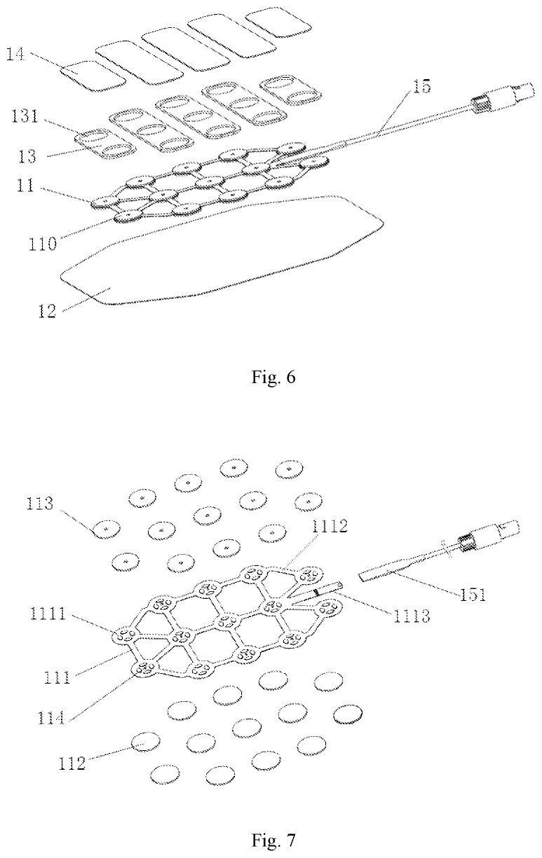

As shown in FIG. 6, the adhesive-backed layer 12 is arranged in a shape of a sheet and is mainly made of a material with flexibility, breathability, insulation, and sterilization. The adhesive-backed layer 12 has multiple ventilation holes (not shown in the figure) penetrating therethrough, so that hair follicles and sweat glands in the skin on the body surface of the patient that is covered by the adhesive-backed layer 12 can breathe freely when the adhesive-backed layer 12 is attached to the body surface of the patient, so as to avoid skin inflammation caused because a superficial layer of the skin of the patient is damaged when the sweat glands and hair follicles on the body surface of the patient covered by the adhesive-backed layer 12 are clogged. The adhesive-backed layer 12 is a mesh fabric. Specifically, the adhesive-backed layer 12 is a mesh-shaped nonwoven fabric. A surface of the adhesive-backed layer 12 facing toward the body surface of the patient is also coated with a biocompatible adhesive (not shown in the figure) for tightly fitting the adhesive-backed layer 12 to a target region on the body surface of the patient.

### A Variant Embodiment of the First Embodiment of the Insulated Electrode

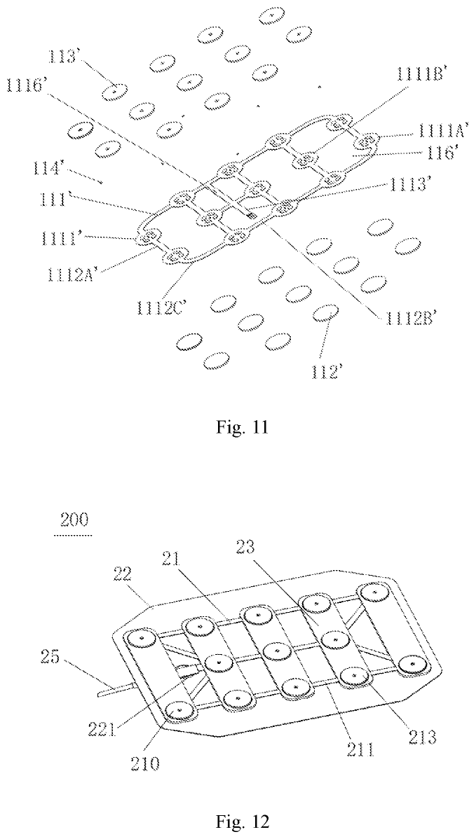

FIG. 10 and FIG. 11 show an insulated electrode 100′ in this embodiment. The insulated electrode is also attached to a body surface in a torso of a patient to perform tumor-treating fields therapy on the tumor site in the torso, and also includes a flexible adhesive-backed layer 12′, an electrical functional component 11′ adhered to the adhesive-backed layer 12′, a plurality of supporting members 13′ adhered to the adhesive-backed layer 12′, a plurality of adhesive members (not shown in the figure) adhered to the supporting members 13′, and a wire 15′ electrically connected to the electrical functional component 11′. The electrical functional component 11′ also includes a flexible circuit board 111′, multiple insulating plates 112′ and multiple dielectric elements 113′ respectively provided on two opposite sides of the flexible circuit board 111′, and multiple temperature sensors 114′ fixed on the flexible circuit board 111′. The main bodies 1111′ of the flexible circuit board 111′, the dielectric elements 113′ and the insulating plates 112′ are arranged in one-to-one correspondence and constitute the electrode elements 110′ of the electrical functional component 11′. The main bodies 1111′ are also distributed in five rows and five columns, and are distributed at the same positions in the array region with five rows and five columns as the main bodies 1111 of the insulated electrode 100 in the first embodiment.

The differences between the insulated electrode 100′ in this embodiment and the insulated electrode 100 in the first embodiment are that: every two peripheral main bodies 1111A′ of the flexible circuit board 111′ are connected with each other through the connecting portions 1112′, and each central main body 1111B′ is only connected to an adjacent peripheral main body 1111A′ in the same row. Specifically, two adjacent peripheral main bodies 1111A′ are electrically connected with each other through either the first connecting portion 1112A′, the second connecting portion 1112B′, or the third connecting portion 1112C′. The peripheral main bodies 1111A′ as well as the first connecting portions 1112A′, the second connecting portions 1112B′ and the third connecting portions 1112C′ each respectively located between corresponding two adjacent peripheral main bodies 1111A′ are in a substantially racetrack shape. Each central main body 1111B′ is connected to the peripheral main body 1111A′ in the same row through the first connecting portion 1112A′. The central main body 1111B′ and the peripheral main body 1111A′ distributed diagonally in adjacent rows and adjacent columns are arranged in a disconnected manner. Two adjacent central main bodies 1111B′ among the three central main bodies 1111B′ are arranged in a disconnected manner. No second connecting portion 1112B′ is arranged between two adjacent central main bodies 1111B′ arranged in the disconnected manner. The third connecting portion 1112C′ is arranged in a shape of an arc. Four third connecting portions 1112C′ are provided, namely, a third connecting portion between the two peripheral main bodies 1111A′ respectively located in the second column of the first row and in the second row of the first column, a third connecting portion between the two peripheral main bodies 1111A′ respectively located in the fourth column of the first row and in the fifth column of the second row, a third connecting portion between the two peripheral main bodies 1111A′ respectively located in the second column of the last row and in the fourth row of the first column, and a third connecting portion between the two peripheral main bodies 1111A′ respectively located in the fourth column of the last row and in the fifth column of the fourth row. The third connecting portion 1112C′ and the adjacent first connecting portion 1112A′ are generally arranged to form an obtuse or acute angle. The third connecting portion 1112C′ and the adjacent second connecting portion 1112B′ substantially form an obtuse angle. The peripheral main body 1111A′ has the same diameter as the central main body 1111B′, and the length of the second connecting portion 1112B′ is slightly larger than the diameter of the peripheral main body 1111A′.

The wiring portion 1113′ is extended laterally from the second connecting portion 1112B′. Specifically, the wiring portion 1113′ is extended laterally from the second connecting portion 1112B′ located between two adjacent central body portions 1111B′. The wiring portion 1113′ and the second connecting portion 1112B′ that extends to form the wiring portion 1113′ are substantially arranged in a shape of “T”. The wiring portion 1113′ and the second connecting portion 1112B′ are arranged perpendicularly. The wiring portion 1113′ and the first connecting portion 1112A′ are arranged substantially in parallel.

The flexible circuit board 111′ also has a reinforcing portion 1116′ opposite to the wiring portion 1113′, so as to provide traction for the wiring portion 1113′ and prevent the insulated electrode 100′ from being affected because of uneven force when the electrode 100′ is attached to the body surface corresponding to the tumor site of the patient. Specifically, the reinforcing portion 1116′ is extended from the second connecting portion 1112B′ which extends laterally to form the wiring portion 1113′. The reinforcing portion 1116′ and the wiring portion 1113′ are respectively located on two opposite sides of the second connecting portion 1112B′ connected to the wiring portion 1113′. The reinforcing portion 1116′ has one end connected to the second connecting portion 1112B′ which is connected with the wiring portion 1113′, and has the other end connected to the second connecting portion 1112B′ adjacent to the second connecting portion 1112B′ connected with the wiring portion 1113′ and located between the two adjacent peripheral main bodies 1111A′. The reinforcing portion 1116′ bridges two adjacent and parallel arranged second connecting portions 1112B′. The reinforcing portion 1116′, the wiring portion 1113′ and the second connecting portion 1112B′ connected with the wiring portion 1113′ are arranged substantially in a shape of chinese character “†”.

The adhesive-backed layer 12′ is provided with a threading hole 121′ corresponding to the wiring portion 1113′ of the flexible circuit board 111′. One end of the wire 15′ passes through the threading hole 121′ and is electrically connected to the wiring portion 1113′. The wire 15′ is extended into the flexible circuit board 111′ from one side of the adhesive-backed layer 12′ and is connected with the wiring portion 1113′, which avoids a problem of reduced comfort of the insulated electrode 100′ which caused by a large number of wires 15′ being directly pressed on the skin of the patient.

The main bodies 1111′ are arranged at intervals, to form a plurality of open spaces 116′ each located between adjacent main bodies 1111′ to allow skin of the body surface corresponding to the tumor site of the patient that is covered by the insulated electrode 100′ to breathe freely after the insulated electrode 100′ is attached to the body surface corresponding to the tumor site of the patient. Sizes of the main body 1111′, the connecting portion 1112′, and the wiring portion 1113′ of the electrical functional component 11′ and a size of the open space 116′ are described in detail below.

All main bodies 1111 of the flexible circuit board 111 are spaced apart within a region with a minimum length of 193 mm and a minimum width of 109 mm. That is, the minimum size of the flexible circuit board 111′ is 193 mm×109 mm. The distance between the main bodies 1111′ in adjacent rows in the flexible circuit board 111′ is greater than the diameter of the main body 1111′, and is at least 22 mm. That is, the length of the second connecting portion 1112B′ is greater than the diameter of the main body 1111′, and is at least 22 mm. The third connecting portion 1112C′ is arranged in a shape of an arc. A length of the third connecting portion 1112C′ is at least 26.7 mm. A width of the reinforcing portion 1116′ is 4.5 mm to 6 mm.

In all the open spaces 116′, the open space 116′ formed by the five main bodies 1111′ located in the first row and in the second row has the largest area. An area of the open space 116′ that is formed by 4 main bodies 1111′ located in alternate columns and adjacent rows of the three middle rows and that accommodates the reinforcing portion 1116′ is the smallest.

The insulated electrodes 100 and 100′ in this embodiment apply an alternating electric field to the tumor site of the patient for tumor treatment through at least 10 electrode elements 110 and 110′ distributed in an array region with at least four rows and three columns, which increases an area of covering the tumor site by the insulated electrodes 100 and 110′, enhances the electric field intensity for the tumor-treating fields therapy, and can further ensure the effect of the tumor-treating fields therapy, thereby making the tumor-treating fields therapy suitable for the torso of the patient. For example, when applied to an abdomen, two insulated electrodes 100 and 100′ are respectively adhered to front and back sides of the waist of the patient, and two insulated electrodes 100 and 100′ are respectively adhered to two sides of the waist of the patient. According to different individual body types, other insulated electrodes 100 and 100′ can also be used. The insulated electrodes 100 and 100′ in this embodiment are more suitable to be attached to a patient with a narrow side waist. In addition, the insulated electrodes 100 and 100′ have multiple open spaces 116 and 116′ arranged at intervals between the main bodies 1111 and 1111′, so that heat accumulated on a skin surface of the patient and water vapor generated in sweating can be discharged into the air during the tumor treatment therapy, which avoids skin inflammation caused due to sweating and clogging of pores in epidermis of the patient to which the insulated electrodes 100 and 100′ are attached and ensure long-duration application of the insulated electrodes 100 and 100′, thereby ensuring the effect of the electric field treatment.

### A Second Embodiment of the Insulated Electrode

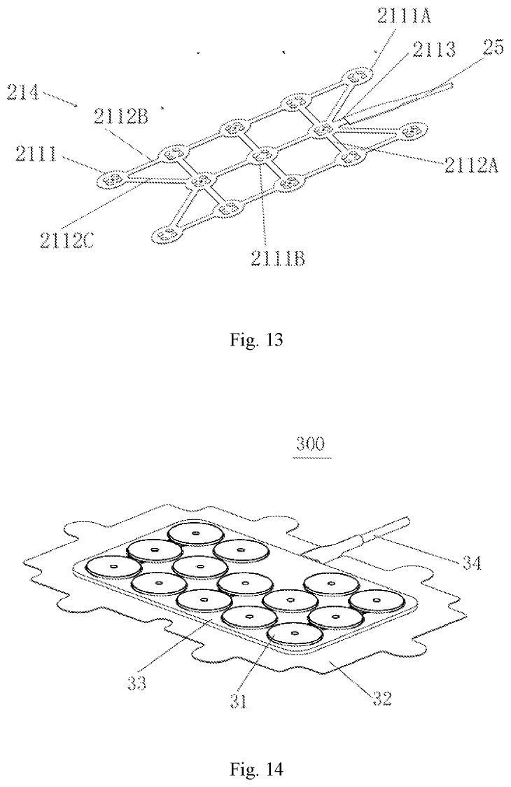

FIG. 12 and FIG. 13 show an insulated electrode 200 according to a second embodiment of the present invention. The insulated electrode is also attached to a body surface in a torso of a patient to perform tumor-treating fields therapy on the tumor site in the torso, and also includes a flexible adhesive-backed layer 22, an electrical functional component 21 adhered to the adhesive-backed layer 22, a plurality of supporting members 23 adhered to the adhesive-backed layer 22, a plurality of adhesive members (not shown in the figure) each adhered to the corresponding supporting member 23, and a wire 25 electrically connected to the electrical functional component 21.

A difference between the insulated electrode 200 in this embodiment and the insulated electrode 100 in the first embodiment is that the electrical functional component 21 of the insulated electrode 200 includes a flexible circuit board 211, multiple insulating plates (not shown in the figure), multiple dielectric elements 213 and multiple temperature sensors 214 arranged on the flexible circuit board 211. Each insulating plate (not shown in the figure) and each dielectric element 213 are respectively arranged on opposite sides of the corresponding main body 2111 of the flexible circuit board 211 in one-to-one correspondence, to form an electrode element 210. The main bodies 2111 are distributed in an array region with five rows and three columns. From the perspective of column arrangement, the first and third columns each is provided with five main bodies 2111, and the second column is provided with three main bodies 2111. Specifically, the two main bodies 2111 located in the first row are respectively located in the first column and the third column. The two main bodies 2111 located in the last row are also respectively located in the first column and the third column. The three main bodies 2111 in each of the three middle rows are respectively loaded in the first column, the second column and the third column. The main bodies 2111 respectively located in the first row and the last row are all arranged in alternate columns, and the main bodies 2111 located in the first row and the last row are all arranged in a disconnected manner. Distances between a plurality of adjacent two main bodies 2111 located in the same row are unequal. Distances between a plurality of adjacent two main bodies 2111 located in the same column are equal. The thirteen main bodies 2111 are arranged axially symmetrically, and has one axis of symmetry overlapped with a straight line on which three main bodies 2111 in the third row are located and another axis of symmetry overlapped with a straight line on which three main bodies 2111 in the second column are located. The thirteen main bodies 2111 are also arranged centrally symmetrically, and have a center of symmetry overlapped with a center of the main bodies 2111 in the third column of the third row. The electrode elements 210 have the same arrangement as the main bodies 2111 and are in an array region with five rows and three columns.

According to distribution positions of the main bodies 2111 in the array, the main bodies 2111 can be divided into twelve peripheral main bodies 2111A located on the periphery of the array and one central main body 2111B surrounded by the peripheral main bodies 2111A and located inside the array. Specifically, one central main body 2111B is the main body 2111 located in the second column of the third row. Twelve peripheral main bodies 2111A are other main bodies 2111 except the main body 2111 located in the second column of the third row. The peripheral main bodies 2111A are connected with each other through either the second connecting portions 2112B or the third connecting portions 2112C. Two adjacent peripheral main bodies 2111A in the same column are connected with each through the corresponding second connecting portion 2112B. Two peripheral main bodies 2111A distributed diagonally in adjacent rows and adjacent columns are connected with each other through the corresponding third connecting portion 2112C. Two adjacent peripheral main bodies 2111A in the same row and alternate columns are arranged in a disconnected manner. Each peripheral main body 2111A and the central main body 2111B are connected with each other through either the first connecting portion 2112A or the second connecting portion 2112B.

Specifically, the peripheral main body 2111A and the central main body 2111B in the same row and adjacent to each other are connected through the corresponding first connecting portion 2112A. The peripheral main body 2111A and the central main body 2111B located in the same column and adjacent to each other are connected through the corresponding second connecting portion 2112B. The first connecting portions 2112A are all located between a plurality of two main bodies 2111 in adjacent columns of the same row and have the same length. The second connecting portions 2112B are located between a plurality of two main bodies 2111 in adjacent rows of the same column and have the same length. The third connecting portion 2112C has a length greater than the length of the first connecting portion 2112A. There are four third connecting portions 2112C, namely, a third connecting portion between the two peripheral main bodies 2111A respectively located in the first column of the first row and in the second column of the second row, a third connecting portion between the two peripheral main bodies 2111A respectively located in the second column of the second row and in the third column of the first row, a third connecting portion between the two peripheral main bodies 2111A in the first column of the fifth row and in the second column of the fourth row, and a third connecting portion between the two peripheral main bodies 2111A in the second column of the fourth row and in the third column of the fifth row. The peripheral main bodies 2111A are arranged axially symmetrically, and has one axis of symmetry overlapped with an extending direction of the row where the central main body 2111B is located and another axis of symmetry overlapped with an extending direction of the column where the central main body 2111B is located. The wiring portion 2113 is arranged by laterally extending the periphery main body 2111A in the second column of the fourth row. The wiring portion 2113 is located between two adjacent third connecting portions 2112C connected to the same peripheral main body 2111A as the wiring portion 2113.

The adhesive-backed layer 22 has a threading hole 221 corresponding to the wiring portion 2113 of the flexible circuit board 211. One end of the wire 25 passes through the threading hole 221 and is electrically connected to the wiring portion 2113. The wire 25 is extended into the flexible circuit board 211 from one side of the adhesive-backed layer 22 and is connected to the wiring portion 2113, which avoids a problem of reduced comfort of the insulated electrode 200 attached to the skin of the patient when a large number of wires 25 are directly pressed on the skin of the patient.

### A Third Embodiment of the Insulated Electrode

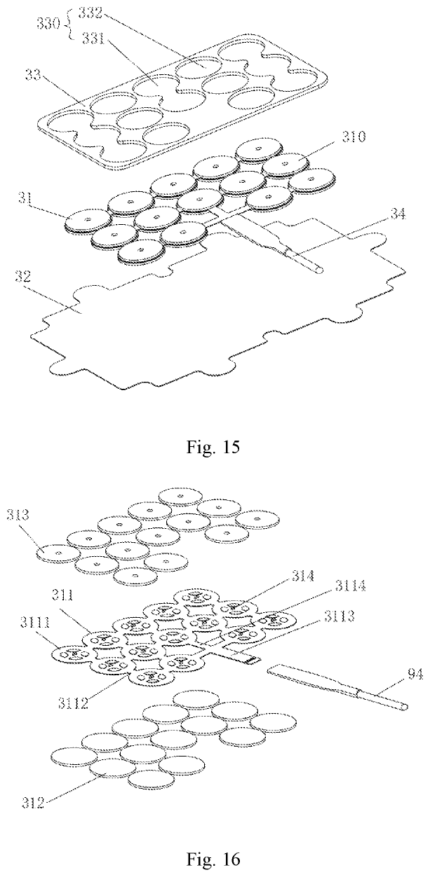

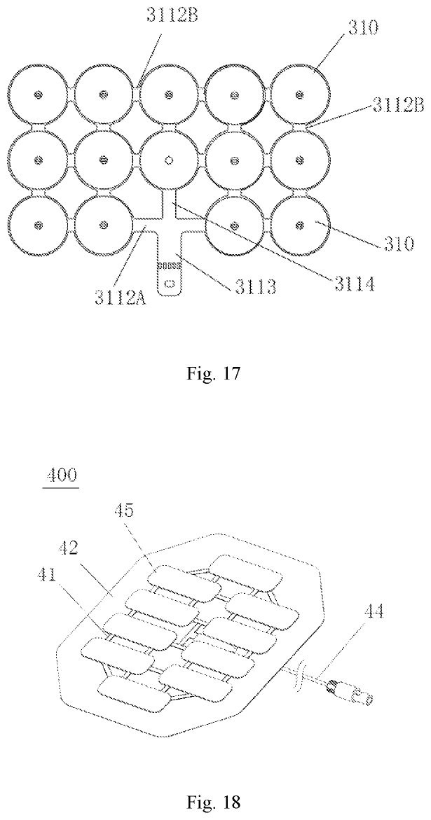

Referring to FIG. 14 to FIG. 17, an insulated electrode 300 in this embodiment includes an adhesive-backed layer 32, an electrical functional component 31 adhered to the adhesive-backed layer 32, a supporting member 33 adhered to the adhesive-backed layer 32, an adhesive member (not shown in the figure) adhered to the adhesive-backed layer 32 and covers corresponding parts of the supporting member 33 and the electrical functional component 31, and a wire 34 electrically connected to the electrical functional component 31.

The electrical functional component 31 includes a plurality of electrode elements 310 arranged substantially in a rectangular array, a plurality of connecting portions 3112 each located between corresponding two adjacent electrode elements 310 and electrically connected with the corresponding two adjacent electrode elements 310, and a wiring portion 3113 extended from one connecting portion 3112. Every two adjacent electrode elements 310 are all connected to each other through the corresponding connecting portion 3112, so as to form the electrical functional component 31 with a grid-shaped structure. The connecting portions 3112 include one first connecting portion 3112A connected with two adjacent electrode elements 310 and also connected with the wiring portion 3113 and a plurality of second connecting portions 3112B each only connected with two adjacent electrode elements 310 in the same row or in the same column. The wiring portion 3113 is laterally extended from the first connecting portion 3112A in a direction away from the electrode element 310 and is electrically connected with the wire 34.

The electrode element 310 includes a main body 3111, an insulating plate 312 arranged on a side of the main body 3111 away from human skin, a dielectric element 313 arranged on a side of the main body 3111 facing toward the human skin, and a temperature sensor 314 that is selectively arranged on the main body 3111 and that is located on the same side as the dielectric element 13. The main bodies 3111, the connecting portions 3112 and the wiring portion 3113 jointly constitute the flexible circuit board 311 of the electrical functional component 31. In this embodiment, the electrode elements 310 of the electrical functional component 31 are arranged more densely to increase the electric field intensity. A specific structure of the electrode element 310 is the same as that in the first embodiment. For details, refer to description in the first embodiment.

The electrical functional component 31 includes a plurality of electrode elements 310 arranged in three rows and five columns, and a plurality of connecting portions 3112 each connected with two adjacent electrode elements 310 in the same row or in the same column. There are fourteen electrode elements 310 in total. From the perspective of row arrangement of the electrode elements 310, the electrode elements 310 include five electrode elements 310 in the first row, five electrode elements 310 in the middle row and four electrode elements 310 in the last row. The connecting portions 3112 each located between two adjacent electrode elements 310 in the first row or in the middle row have the same length between 1 mm and 3 mm, preferably 2.1 mm. The connecting portions 3112 each located between two adjacent electrode elements 310 in the last row have different lengths. The length of the connecting portion 3112 located between two adjacent electrode elements 310 in adjacent columns of the last row is equal to the length of the connecting portion 3112 located between two adjacent electrode elements 310 in the first row or in the middle row. The length of the connecting portion 3112 between two adjacent electrode elements 310 in adjacent columns of the last row is less than the length of the connecting portion 3112 between two adjacent electrode elements 310 in spaced columns of the last row. The length of the connecting portion 3112 between two adjacent electrode elements 310 in adjacent columns of the last row is between 1 mm and 3 mm, preferably 2.1 mm. The length of the connecting portion 3112 between two adjacent electrode elements 310 in spaced columns of the last row is between 22 mm and 27 mm.

From the perspective of column arrangement of the electrode elements 310, there are only two electrode elements 310 in the middle column, and three electrode elements 310 in each of the other four columns. The connecting portions 3112 connected with two adjacent electrode elements 310 in each column have the same length and are equal to the length of the connecting portion 3112 connecting two adjacent electrode elements 310 in the first or middle row. Lengths of the connecting portions 3112 connected with two adjacent electrode elements 310 in each column are between 1 mm and 3 mm, preferably 2.1 mm. The lengths of the connecting portions 3112 between two adjacent electrode elements 310 arranged in columns are all the same and are between 1 mm and 3 mm, preferably 2.1 mm. The lengths of the connecting portions 3112 between two adjacent electrode elements 310 arranged in rows are different. The length of the connecting portion 3112 connected with two electrode elements 310 in adjacent columns of the same row is less than the length of the connecting portion 3112 connected with two electrode elements 310 in spaced columns of the same row. The connecting portions 3112 between two adjacent electrode elements 310 in adjacent rows of the same column are all the second connecting portions 3112. The connecting portions 3112 between two adjacent electrode elements 310 in adjacent columns of the same row are also the second connecting portions 3112. Lengths of the second connecting portions are between 1 mm and 3 mm, preferably 2.1 mm. The connecting portions 3112 between two adjacent electrode elements 310 in spaced columns of the same row are the first connecting portions 3112. The first connecting portion 3112A and the second connecting portion 3112B are both arranged in a shape of “−”. The length of the first connecting portion 3112A and the length of the second connecting portion 3112B are different. The length of the first connecting portion 3112A is greater than the length of the second connecting portion 3112B.

The flexible circuit board 311 of the electrical functional component 31 may also include a reinforcing portion 3114. The reinforcing portion 3114 has one end connected to the first connecting portion 3112A and the other end connected to the electrode element 310 corresponding to the first connecting portion 3112A. The reinforcing portion 3114 and the wiring portion 3113 are on the same straight line. The reinforcing portion 3114 and the first connecting portion 3112A are arranged perpendicularly. The reinforcing portion 3114 and the first connecting portion 3112A are arranged in a shape of “F” or “T”. The reinforcing portion 3114 and the wiring portion 3113 are respectively located at two opposite sides of the first connecting portion 3112A. The reinforcing portion 3114 can enhance strength of the wiring portion 3113 arranged opposite thereto and can provide traction for the wiring portion 3113, thereby preventing uneven force from affecting the application of the insulated electrode 300 when the insulated electrode 300 is attached to the body surface corresponding to the tumor site of the patient. The length of the reinforcing portion 3114 is not less than the length of the second connecting portion 3112B. That is, the length of the reinforcing portion 3114 is greater than or equal to the length of the second connecting portion 3112B connected with two adjacent electrode elements 310 in adjacent columns of the same row, or greater than or equal to the length of the second connecting portion 3112B connected with two adjacent electrode elements 310 in adjacent rows of the same column.

In this embodiment, the electrode element 310 has a substantially circular sheet-shaped structure and a diameter of the electrode element 310 is approximately 21 mm. The length of the second connecting portion 3112B is 1 mm to 3 mm, which can increase the number of the electrode elements 310 of the insulated electrode 300 per unit area, increase a coverage area of the electrode elements 310 of the insulated electrode 300 without increasing the overall area of the insulated electrode 300, enhance the intensity of the electric field applied to the tumor site for TTF treatment, and increase a range of covering the tumor site by the alternating electric field, thereby improving a therapeutic effect.

In this embodiment, the wiring portion 3113 has a corresponding part closer to the connecting portion 3112 and located between the two electrode elements 310 in the middle of the last row, so as to use space between the electrode elements 310 to shorten a length of a part thereof that exceeds the edge of the electrode element 310, thereby avoiding increasing manufacturing costs caused by an excessively large overall size of the electrical functional component 31. The wiring portion 3113 and the electrode element 310 adjacent to the wiring portion 3113 are arranged at intervals, which can provide a larger operating space for welding the wiring portion 3113 and the wire 34. To facilitate arrangement of conductive traces (not shown in the figure), the wiring portion 3113 is wider than the connecting portion 3112. Preferably, a width of the connecting portion 3112 is 4 mm to 6 mm, and a width of the wiring portion 3113 is 7 mm to 9 mm. In this embodiment, the width of the connecting portion 3112 is 4.5 mm, and the width of the wiring portion 3113 is 8 mm. It can be understood that some connecting portions 3112 may not be used for providing the conductive traces (not shown in the figure), but are only used to increase the strength of the flexible circuit board 11.

In this embodiment, the supporting member 33 is a single piece of foam. The supporting member 33 is provided with a plurality of through-holes 330 corresponding to the electrode elements 310 of the electrical functional component 31, to accommodate the corresponding electrode elements 310. The supporting member 33 surrounds each electrode element 310 of the electrical functional component 31, which can improve the overall strength of the insulated electrode 300. The through-holes 330 include multiple first through-holes 331 and multiple second through-holes 332. The multiple first through-holes 331 communicate with each other and surround the multiple electrode elements 310 arranged in columns, and can accommodate the connecting portions 3112 connected with two adjacent electrode elements 310 in the same column, thereby reducing contact between the supporting member 33 and the connecting portion 3112 of the electrical functional component 31, so that the supporting member 33 can be adhered to the adhesive-backed layer 32 more smoothly. The multiple second through-holes 332 are provided on the supporting member 33 at intervals, and each respectively surrounds one corresponding electrode element 310 arranged in columns. In this embodiment, the multiple first through-holes 331 respectively surrounds three electrode elements 310 in the first column, two electrode elements 310 in the third column, and three electrode elements 310 in the fifth column. The multiple second through-holes 332 surround each electrode element 310 in the second column and the fourth column respectively. The multiple second through-holes 332 are arranged in columns, and the multiple second through-holes 332 arranged in the columns are arranged at intervals to ensure strength of the supporting member 33 and avoid breakage due to external force. The first through-hole 331 is substantially in a shape of a racetrack.

The adhesive member (not shown in the figure) is a single piece, and has a size slightly larger than the size of the supporting member 33. The adhesive member (not shown in the figure) is preferably conductive gel. The adhesive member (not shown in the figure) has adhesiveness on both sides, and can keep a skin surface moist and relieve local pressure when in contact with the skin.

The insulated electrode 300 may also be covered with release paper (not shown in the figure) on outer sides of the adhesive member (not shown in the figure) and the adhesive-backed layer 32 to protect the adhesive member (not shown in the figure) and the adhesive-backed layer 32 and prevent the adhesive member (not shown in the figure) and adhesive-backed layer 32 being stained. The insulated electrode 300 can be covered by only one piece of release paper (not shown in the figure) located on the adhesive member (not shown in the figure) and the adhesive-backed layer 32, or can be jointly covered by two or more pieces of release paper (not shown in the figure) located on the adhesive member (not shown in the figure) and adhesive-backed layer 32. During use, the release paper (not shown in the figure) is torn off and the insulated electrode 300 is adhered to the body surface corresponding to the tumor site of the human body.

The insulated electrode 300 in this embodiment applies the alternating electric field to the tumor site of the patient through the fourteen electrode elements 310 provided thereon for tumor treatment, which can prevent the therapeutic effect from being affected due to insufficient electric field treatment caused by differences in tumor sizes, sites, and positions, increase the coverage area of the electrode elements 310 of the insulated electrode 300, enhance the intensity of the electric field applied to the tumor site for the TTF treatment, and increase a range of covering the tumor site by the alternating electric field, thereby improving the therapeutic effect.

### A Fourth Embodiment of the Insulated Electrode

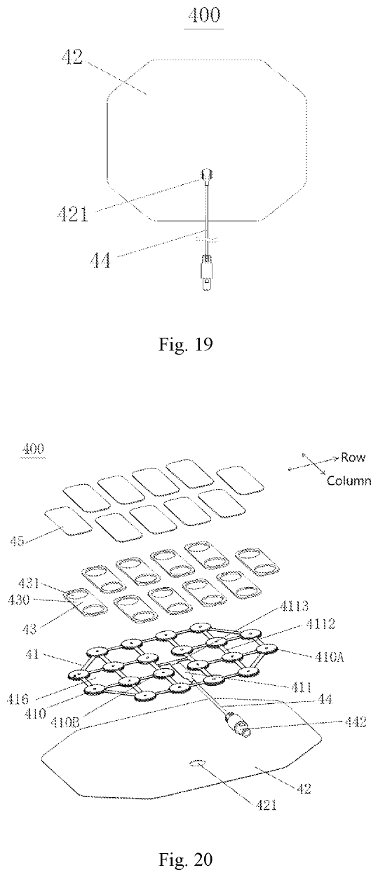

Referring to FIG. 18 to FIG. 21, an insulated electrode 400 in this embodiment includes an adhesive-backed layer 42, an electrical functional component 41 adhered to the adhesive-backed layer 42, a plurality of support members 43 each adhered to the adhesive-backed layer 42, a plurality of adhesive members 45 each covering corresponding parts of the supporting member 43 and the electrical functional component 41, and a wire 44 electrically connected to the electrical functional component 41. The insulated electrode 400 is attached to the body surface corresponding to the tumor site of the patient through the adhesive-backed layer 42, and applies an alternating electric signal to the tumor site of the patient through the electrical functional component 41 to interfere with or prevent mitosis of tumor cells of the patient, thereby achieving a tumor treatment purpose.

The electrical functional component 41 is arranged in a shape of a grid, and includes multiple electrode elements 410 arranged in an array, multiple connecting portions 4112 each connected with two adjacent electrode elements 410, and a wiring portion 4113 welded to a wire 44. The multiple electrode elements 410 are distributed at intervals on grid points of the electrical functional component 41. Each electrode element 410 is connected with at least two electrode elements 410 adjacent thereto through the corresponding connecting portions 4112. Each electrode element 410 is connected to at least two connecting portions 4112. There are at least ten electrode elements 410 distributed in an array region with at least three rows and four columns, which can increase a coverage area of the electrode elements 410 of the insulated electrode 400, enhance the intensity of the electric field applied to the tumor site for tumor-treating fields therapy, and increase a range of covering the tumor site by the alternating electric field, thereby improving a therapeutic effect.

Preferably, each electrode element 410 is connected with at least three electrode elements 410 adjacent thereto through corresponding connecting portions 4112. Each electrode element 410 is connected with at least three connecting portions 4112. There are twenty electrode elements 410 distributed in an array region with four rows and six columns. Numbers of electrode elements 410 in columns are not completely the same. Numbers of electrode elements 410 in rows may be completely the same or may be not completely the same. At least two adjacent electrode elements 410 in the multiple electrode elements 410 are arranged in a disconnected manner, and a gap 4C for allowing the wiring portion 4113 to pass through is formed between the two adjacent electrode elements 410 in the disconnected manner. The wiring portion 4113 is laterally extended from the connecting portion 4112 opposite to the gap 4C. The connecting portion 4112 that extends to form the wiring portion 4113 is arranged perpendicular to the wiring portion 4113. The connecting portion 4112 which extends to form the wiring portion 4113 and the wiring portion 4113 are substantially arranged in a shape of “T”. The wiring portion 4113 is arranged substantially in a shape of “−”. Optionally, the wiring portion 4113 is arranged in a shape of “T” and bridges two connecting portions 4112 that are respectively connected with the two adjacent electrode elements 410 arranged in a disconnected manner. The wiring portion 4113 is located between the multiple electrode elements 410 and is arranged in the space surrounded by the multiple electrode elements 410, which can avoid an increase in manufacturing costs due to an excessively large overall size of the electrical functional component 41.

Twenty electrode elements 410 are arranged in the array region with four rows and six columns in such a manner that two electrode elements 410 are arranged in each of two columns, and four electrode elements are arranged in each of the other four columns. Specifically, the twenty electrode elements 410 are arranged in the array region with four rows and six columns in such a manner that each two of four columns each includes four electrode elements 410 are arranged in adjacent manner. Distances between a plurality of two adjacent electrode elements 410 arranged in rows are the same. The connecting portions 4112 each connects two adjacent electrode elements 410 in rows have the same length. Specifically, the electrode elements 410 in each of the two columns each of which includes only two electrode elements 410 are arranged in adjacent rows. Distances between two adjacent electrode elements 410 arranged in columns are the same, and the connecting portions 4112 each connecting two adjacent electrode elements 410 in columns have the same length. The four electrode elements 410 in the two columns can be aligned in a row direction respectively, or can also be arranged at intervals in the row direction, or two electrode elements 410 in one column of the two columns can be aligned in the row direction and two electrode elements 410 in the other one column of the two columns can be arranged at intervals in the row direction. Optionally, the two electrode elements 410 in at least one of the two columns each of which is provided with only two electrode elements 410 are arranged in alternate rows. Distances between two electrode elements 410 in columns are different, and the connecting portions 4112 each connecting two adjacent electrode elements 410 in columns have different lengths.

Optionally, the twenty electrode elements 410 are arranged in the array region with four rows and six columns in such a manner that at least two of four columns each of which is provided with four electrode elements 410 are arranged in spaced columns. Distances between two adjacent electrode elements 410 arranged in rows are different, and the connecting portions 4112 each connecting two adjacent electrode elements 410 in rows have different lengths. Specifically, the two electrode elements 410 in at least one of the two columns each of which is provided with only two electrode elements 410 are arranged in spaced rows. Distances between two adjacent electrode elements 410 in columns are different, and the connecting portions 4112 each connecting two adjacent electrode elements 410 in columns have different lengths. Optionally, the two electrode elements 410 in each of the two columns each of which includes only two electrode elements 410 are arranged in adjacent rows. Distances between two adjacent electrode elements 410 in columns are the same, and the connecting portions 4112 each connecting two adjacent electrode elements 410 in columns have the same length.

Optionally, the twenty electrode elements 410 are distributed in the array region with four rows and six columns in such a manner that one column is provided with one electrode element 410, one column is provided with three electrode elements 410, and the rest four columns each is provided with four electrode elements 410. Specifically, the twenty electrode elements 410 are distributed in the array region with four rows and six columns in such a manner that one electrode element 410 is arranged in the first column, four electrode elements 410 are arranged in each of the middle four columns, and three electrode elements 410 adjacent to each other are arranged in the last column. Distances between two adjacent electrode elements 410 in rows are the same, and distances between two adjacent electrode elements 410 in columns are the same. That is, the connecting portions 4112 each connects two adjacent electrode elements 410 in the same row have the same length. Multiple connecting portions 4112 each connecting two adjacent electrode elements 410 in the same column have the same length.

Optionally, the twenty electrode elements 410 are distributed in the array region with four rows and six columns in such a manner that one electrode element 410 is arranged in the first column, four electrode elements 410 are arranged in each of the middle four columns, and two adjacent electrode elements 410 in three electrode elements 410 in the last column are arranged at intervals. Distances between two adjacent electrode elements 410 arranged in rows are the same, and distances between two adjacent electrode elements 410 in columns are different. That is, multiple connecting portions 4112 each connected with two adjacent electrode elements 410 in the same row have the same length. That is, multiple connecting portions 4112 each connecting two adjacent electrode elements 410 in the same column have different lengths.

Optionally, the twenty electrode elements 410 are distributed in the array region with four rows and six columns in such a manner that each of the first column to the fourth column is provided with fourth electrode elements 410, the fifth column is provided with three electrode elements 410, and the last column is provided with only one electrode element 410. The electrode element 410 in the last column and one of the three electrode elements 410 in the fifth column are aligned in the row direction, and the three electrode elements 410 in the fifth column are arranged adjacently in the row direction. Distances between two adjacent electrode elements 410 arranged in rows are the same, and distances between two adjacent electrode elements 410 arranged in columns are the same. Multiple connecting portions 4112 each connecting two adjacent electrode elements 410 in rows have the same length, and multiple connecting portions 4112 each connecting two adjacent electrode elements 410 in columns have the same length. Optionally, the electrode element 410 in the last column and the three electrode elements 410 in the fifth column are staggered in the row direction, and the three electrode elements 410 in the fifth column are arranged adjacently in the row direction. Distances between two adjacent electrode elements 410 arranged in rows are different, and distances between two adjacent electrode elements 410 arranged in columns are the same. Multiple connecting portions 4112 each connects two adjacent electrode elements 410 in rows have different lengths, and multiple connecting portions 4112 each connects two adjacent electrode elements 410 in columns have the same length. Optionally, the electrode element 10 in the last column and the three electrode elements 410 in the fifth column are staggered in the row direction, and two adjacent electrode elements 410 in the three electrode elements 410 in the fifth column are arranged in spaced rows. Distances between two adjacent electrode elements 410 arranged in rows are different, and distances between two adjacent electrode elements 410 arranged in columns are different. Multiple connecting portions 4112 each connects two adjacent electrode elements 410 arranged in rows have different lengths, and multiple connecting portions 4112 each connects two adjacent electrode elements 410 arranged in columns have different lengths.

Optionally, the twenty electrode elements 410 are distributed in the array region with four rows and six columns in such a manner that at least two of four columns each of which is provided with four electrode elements 410 are arranged at intervals. Distances between two adjacent electrode elements 410 arranged in rows are different. Multiple connecting portions 4112 each connecting two adjacent electrode elements 410 arranged in rows have different lengths. Distances between two adjacent electrode elements 410 arranged in columns may be the same or may be different. Multiple connecting portions 4112 each connects two adjacent electrode elements 410 arranged in columns may have the same length or may have different lengths.

The twenty electrode elements 410 in this embodiment are arranged in the array region with four rows and six columns in such a manner that four electrode elements 410 are arranged in each of the first row and the last row, and six electrode elements 410 are arranged in each of the middle two columns. From the perspective of column arrangement, two electrode elements 410 are arranged in each of the first column and sixth column, and four electrode elements 410 are arranged in each of the middle four columns. The electrode elements 410 of the first column and the sixth column located in the same column are arranged adjacently in the row direction, and the electrode elements 410 of the first and sixth columns are respectively arranged in a row-aligned manner. Specifically, the four electrode elements 410 in the first row are respectively located in the columns from the second column to the fifth column, the six electrode elements 410 in each of the middle two rows are respectively located in the columns from the first column to the sixth column, and the four electrode elements 410 in the last row are respectively located in the columns from the second column to the fifth column. The multiple electrode elements 410 of the electrical functional component 41 are axial-symmetrically arranged. The multiple electrode elements 410 of the electrical functional component 41 are both axial-symmetrically arranged in the row direction and axial-symmetrically arranged in the column direction. The twenty electrode elements 410 are arranged in an octagonal shape.

The connecting portions 4112 connect all two adjacent electrode elements 410 located at the periphery of the array, and at least one pair of the two adjacent electrode elements 410 among the two adjacent electrode elements 410 in the inner layer of the array are arranged in a disconnected state. Specifically, the connecting portions 4112 are located between all adjacent electrode elements 410 except the two electrode elements 410 located in the third column of the second row and the fourth column of the second row, and two electrode elements 410 located in the third column of the third row and the fourth column of the third row. The lengths of the connecting portions 4112 connecting two adjacent electrode elements 410 arranged in rows are equal. The lengths of the connecting portions 4112 connecting two adjacent electrode elements 410 arranged in columns are equal. The connecting portions 4112 are located between two adjacent electrode elements 410 arranged in rows, between two electrode elements 410 arranged in columns, and between two adjacent electrode elements 410 diagonally arranged in adjacent rows and adjacent columns and located at the periphery of the array.

From the perspective of distribution positions of the electrode elements 410 in the array, the plurality of electrode elements 410 can be divided into a plurality of peripheral electrode elements 410A located at the periphery and a plurality of central electrode elements 410B surrounded by the peripheral electrode elements 410A. In this embodiment, there are twelve peripheral electrode elements 410A and 8 central electrode elements 410B. All the peripheral electrode elements 410A are connected with each other through the corresponding connecting portions 4112. That is, the connecting portions 4112 are arranged between all the adjacent peripheral electrode elements 410A. At least two adjacent central electrode elements 410B located in the same row or the same column are arranged in a disconnected manner, so as to form a gap 4C therebetween to allow the wiring portion 4113 to pass through.

The gaps 4C are arranged between two adjacent electrode elements 410 located in the third column of the second row and the fourth column of the second row, and two adjacent electrode elements 410 located in the third column of the third row and the fourth column of the third row. The wiring portion 4113 is located between the electrode element 410 of the third column and the electrode element 410 of the fourth column. The wiring portion 4113 is substantially arranged in a shape of “T” and passes through the gap 4C, and bridges the connecting portion 4112 located between the two adjacent electrode elements 410 in the middle of the third column and the connecting portion 4112 located between the two adjacent electrode elements 410 in the middle of the fourth column. The wiring portion 4113 and the two adjacent connecting portions 4112 connected to the wiring portion 4113 are axial-symmetrically arranged. Optionally, the wiring portion 4113 is arranged in a shape of the Chinese character “−”, and is laterally extended from the connecting portion 4112 corresponding to the gap 4C toward the gap 4C.

The wiring portion 4113 of the electrical functional component 41 is electrically connected to the wire 44. In this embodiment, rows of golden fingers 41130 welded to the wires 44 are separately staggered on two side surfaces of an end of the wiring portion 4113 that is away from the connecting portion 4112 to which the wiring portion 4113 is connected. The wire 44 has one end electrically connected with the golden fingers 41130 of the wiring portion 4113 and the other end electrically connected to an electric field generator (not shown in the figure) through the plug 42 thereof, so as to provide the insulated electrode 400 with an alternating electric signal for tumor treatment during tumor-treating fields therapy. A heat shrinkable sleeve 41 is wrapped around a welded joint of the wire 44 and the golden finger 41130 of the wiring portion 4113. The heat shrinkable sleeve 41 insulates and protects the joint between the wire 44 and the wiring portion 4113 of the electrical functional component 41 and provides support, thereby avoiding breakage of the joint between the wire 44 and the wiring portion 4113 of the electrical functional component 41 and further achieving dust-proof and waterproof purposes.

The electrode element 410 includes a main body 4111 arranged at two opposite ends of the connecting portion 4112, an insulating plate 412 arranged on a side of the main body 4111 that is away from human skin, a dielectric element 413 arranged on a side of the main body 4111 that faces toward the human skin, and a temperature sensor 414 that is selectively arranged on the main body 4111 and located at the same side as the dielectric element 413. The main body 4111, the insulating plate 412 and the dielectric element 413 are all circular sheet-shaped structures. The insulating plate 412, the main body 4111 and the dielectric element 413 are arranged in one-to-one correspondence along a thickness direction, and have centers located on the same straight line. In other embodiments, the main body 4111 may also be an intersection-shaped structure arranged by extending end of the corresponding connecting portion 4112.

A conductive pad 4114 is arranged on one surface of the main body 4111 facing toward the dielectric element 413. The conductive pad 4114 of the main body 4111 can be completely covered by the dielectric element 413, so that the conductive pad 4114 and the dielectric element 413 can be welded through soldering tin (not shown in the figure). The conductive pad 4114 of the main body 4111 includes multiple conductive cores 41140 central-symmetrically arranged, which can effectively prevent the offset of the position of the dielectric element 413 caused by the stacking of soldering tin (not shown includes in the figure) during the welding process. The center of the conductive pad 4114 of the main body 4111 is located on a center line of the main body 4111. Top surfaces of multiple conductive cores 41140 of the conductive pad 4114 are located in the same plane, so that the virtual welding between the conductive cores 41140 and the dielectric element 413 can be avoided. The center of the conductive pad 4114 is also located on a center line of the corresponding dielectric element 413.

In this embodiment, the conductive pad 4114 located on the same main body 4111 includes four conductive cores 41140 center-symmetrically arranged at intervals. The conductive cores 41140 are arranged in a multi-point interval mode, which can reduce the consumption of copper foil used to manufacture the conductive cores 41140 and reduce material costs. In addition, this can also reduce the amount of soldering tin (not shown in the figure) used to weld the conductive cores 41140 and the corresponding dielectric element 413, thereby further reducing the material costs. The four conductive cores 41140 of the same conductive pad 4114 all have petal-shaped structures. Each conductive core 41140 includes an inner arc (not numbered) and an outer arc (not numbered) connected end to end. The inner arc (not numbered) and the outer arc (not numbered) of the conductive core 41140 are axial-symmetrically arranged. The inner arcs (not numbered) of the four conductive cores 41140 of the same conductive pad 4114 are all recessed toward the center of the conductive pad 4114. The outer arcs (not numbered) of the four conductive cores 41140 of the same conductive pad 4114 all protrude in a direction away from the center of the conductive pad 4114. The four conductive cores 41140 constituting the conductive pad 4114 are both center-symmetrically and axial-symmetrically arranged, and each conductive core 41140 is also axial-symmetrically arranged. Therefore, when the four conductive cores 41140 of the conductive pad 4114 of the main body 4111 are welded with the dielectric element 413, the stress balance between each welding point between the conductive pad 4114 and the dielectric element 413 is guaranteed, an overall welding balance of the dielectric element 413 is ensured, and the welding quality is improved, so as to avoid an inclination of the dielectric element 413 caused by the unbalanced welding stress, which may result in a weak strength and easy fracture of the welding point on a side with larger spacing between the dielectric element 413 and the main body part 4111. At the same time, it may also avoid affecting the fit of the insulated electrode 400.

The insulating plate 412 is made of an insulating material. Preferably, the insulating plate 412 is an epoxy glass cloth laminate plate. The insulating plate 412 is adhered to a surface of the main body 4111 that is away from human skin through the sealant (not shown in the figure), which provide a flat welding plane for a welding operation between the main body 4111 and the dielectric element 413 while enhancing the strength of the main body 4111, thereby improving product yield rate. In addition, the insulating plate 412 can further isolate the water vapor in the air on the side of the insulated electrode 400 that is away from the skin from the soldering tin (not shown in the figure) located between the main body 4111 and the dielectric element 413, and further prevent an electrical connection between the main body 4111 and the dielectric element 413 being affected because the water vapor corrodes the soldering tin (not shown in the figure) between the main body 4111 and the dielectric element 413.

The size of the insulating plate 412 is the same as that of the main body 4111, so as to prevent the sealant (not shown in the figure) from climbing to the side of the main body part 4111 facing the human skin by capillary effect when the insulating plate 412 is adhered to a surface of the main body part 4111 away from human skin by sealant (not shown in the figure), which may affect the filling of the sealant (not shown in the figure) in a gap (not shown in the figure) formed by welding the dielectric element 413 and the main body part 4111, resulting the existence of cavities in the sealant (not shown in the figure). This may further prevent the sealant (not shown in the figure) from bursting due to the rapid expansion of water vapor caused by a large difference in thermal expansion coefficient between the sealant (not shown in the figure) and the water vapor in the cavities during high-temperature curing, which may lead to popcorn phenomenon, and damage the products.

The dielectric element 413 is made of a material with a high dielectric constant which has a property of impeding conduction of the direct current and allowing conduction of the alternating current, thereby ensuring safety of human body. Preferably, the dielectric element 413 is a dielectric ceramic plate. The dielectric element 413 has a ring-shaped structure, and has a through-hole 4132 extending through the middle thereof, to accommodate the temperature sensor 414. A ring-shaped metal layer (not shown in the figure) is attached to the surface of the dielectric element 413 facing toward the main body 4111. A Point-to-surface welding is formed between the metal layer (not shown in the figure) of the dielectric element 413 and the conductive cores 41140 of the conductive pad 4114 of the main body 4111, so that it is more convenient to weld without requiring high welding alignment accuracy. The gap (not shown in the figure) formed by welding between the dielectric element 413 and the main body 4111 is filled with the sealant (not shown in the figure) to protect the soldering tin (not shown in the figure) between the dielectric element 413 and the main body 4111, thereby preventing the alternating electric field being unable to be applied to the tumor site of the patient through the dielectric element 413 because the welding joint is broken when the dielectric element 413 is affected by external force, and further ensuring that the dielectric element 413 is fixed onto the main body 4111 through the sealant (not shown in the figure). The inner ring of the metal layer (not shown in the figure) of the dielectric element 413 and the through-hole 4132 of the dielectric element 413 are arranged at intervals, which can avoid a short circuit of the temperature sensor 414 that is caused because the soldering tin (not shown in the figure) between the metal layer (not shown in the figure) of the dielectric element 413 and the main body 4111 is melted and spreads in the direction of approaching the through-hole 4132 of the dielectric element 413 when heated. The outer ring of the metal layer (not shown in the figure) of the dielectric element 413 and the outer edge of the dielectric element 413 are also arranged at intervals, which can prevent the soldering tin (not shown in the figure) between the metal layer (not shown in the figure) of the dielectric element 413 and the main body 4111 from melting and overflowing to the outer side of the main body 4111 when heated, and prevent the direct current unimpeded by the dielectric element 413 from passing through and directly acting on the body surface of the patient when the insulated electrode 400 is attached to the body surface corresponding to the tumor site of the patient.

The outer diameter of the dielectric element 413 is slightly less than the diameter of the main body 4111, and the sealant (not shown in the figure) is filled into the gap (not shown in the figure) along the edge of the main body 4111 located outside the dielectric element 413 through a capillary phenomenon, so as to facilitate the filling of the sealant (not shown in the figure) into the gap (not shown in the figure) formed by welding the dielectric element 413 with the main body 4111. With reference to the through-hole 4132 of the dielectric element 413, when the gap (not shown in the figure) formed by welding the dielectric element 413 with the main body 4111 is filled with the sealant (not shown in the figure), the air in the gap (not shown in the figure) can be exhausted through the through-hole 4132 of the dielectric element 413, so as to prevent the sealant (not shown in the figure) filled in the gap (not shown in the figure) from generating cavities, thereby improving quality of the product.

Multiple temperature sensors 414 are provided and respectively accommodated in the through-holes 4132 of the corresponding dielectric elements 413. In this embodiment, the number of temperature sensors 414 is eight, respectively arranged on eight electrode elements 410 respectively located in the third column of the first row, in the fourth column of the first row, in the third column of the last row, in the fourth column of the last row, in the second column of the second row, in the fifth column of the second row, in the second column of the third row, and in the fifth column of the third row. Eight temperature sensors 414 are respectively arranged at centers of the main bodies 4111 of the corresponding electrode elements 410. Each temperature sensor 414 is configured to monitor the temperature of the surface of the corresponding dielectric element 413 of the electrical functional component 41 facing toward the human skin, so as to detect temperature of the human skin to which the corresponding adhesive member 45 is attached. When the temperature monitored by the temperature sensor 414 exceeds an upper safe temperature limit of the human body, the electric field generator (not shown in the figure) promptly reduces or cuts off the alternating current transmitted to the insulated electrode 400, so as to avoid a low-temperature burn of the human body. The temperature sensor 414 is welded to the main body 4111 and then sealed with the sealant (not shown in the figure) to reliably fix the temperature sensor 414 and also prevent water vapor from corroding the temperature sensor 414 and causing the temperature sensor 414 to fail. The temperature sensor 414 has a signal end (not shown in the figure) and a ground end (not shown in the figure). In other embodiments, the specific number of temperature sensors 14 can be set as required. The temperature sensor 414 is preferably a thermistor.

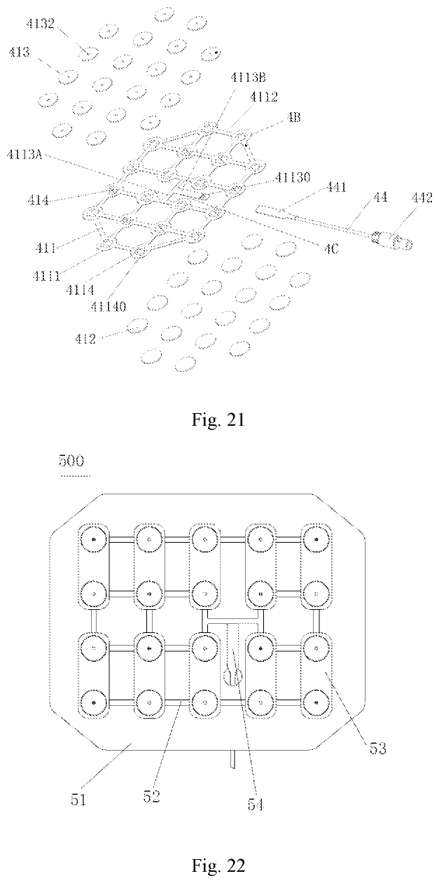

As shown in FIG. 21, the main bodies 4111 of the electrode elements 410 arranged in four rows and six columns, the connecting portions 4112 each connecting two adjacent electrode elements 420 and the wiring portion 4113 bridging two adjacent connecting portions 4112 jointly form the flexible circuit board 411 of the electrical functional component 41. The flexible circuit board 411 is arranged in a shape of a grid. Each dielectric element 413 is arranged at a grid point of the flexible circuit board 411. Each main body 4111 can be considered as the grid point of the flexible circuit board 411. From the perspective of formation of the electrode element 410, the insulating plate 412 is arranged on the side of the main body 4111 of the flexible circuit board 411 that is away from the human skin, the dielectric element 413 is arranged on the side of the main body 4111 of the flexible circuit board 411 facing toward the human skin, and the temperature sensor 414 is selectively arranged on the side of the main body 4111 of the flexible circuit board 411 facing toward the human skin. Arrangement of the main body 4111 of the flexible circuit board 411 is consistent with that of the electrode element 410.

The flexible circuit board 411 includes an insulating substrate 4B and multiple conductive traces (not shown in the figure) embedded in the insulating substrate 4B. Conductive traces (not shown in the figure) embedded in the insulating substrate 4B of the main body 4111, conductive traces (not shown in the figure) embedded in the insulating substrate 4B of the connecting portion 4112, and the conductive traces (not shown in the figure) embedded in the insulating substrate 4B of the wiring portion 4113 are electrically connected. Conductive traces (not shown in the figure) are embedded in insulating substrates 4B of some connecting portions 4112, and other connecting portions 4112 only include the insulating substrates 4B to enhance strength of the flexible circuit board 411. The conductive cores 41140 are exposed or protrude from the insulating substrate 4B of the main body 4111. The insulating substrate 4B of the flexible circuit board 411 can isolate water vapor in the air around the insulated electrode 400 from the soldering tin (not shown in the figure) between the conductive core 41140 of the conductive pad 4114 on the main body 4111 of the flexible circuit board 411 and the dielectric element 413, and prevent water vapor in the air on the side away from the skin corroding the soldering tin (not shown in the figure) between the main body 4111 and the dielectric element 413 of the flexible circuit board 411. The insulating substrate 4B of the flexible circuit board 411 and the insulating plate 412 have a function of double isolation, which can prolong service life of the insulated electrode 400. The golden fingers 41130 of the wiring portion 4113 are exposed from the insulating substrate 4B.

The conductive traces (not shown in the figure) of the flexible circuit board 411 include a conductive trace (not shown in the figure) that connects all the conductive cores 41140 of the conductive pad 4114 of each main body 4111 in series, a conductive trace (not shown in the figure) that connects grounding ends (not shown in the figure) of each temperature sensor 414 on the corresponding main body 4111 in series, and multiple conductive traces (not shown in the figure) electrically and respectively connected to signal ends (not shown in the figure) of the temperature sensors 414 on the corresponding main bodies 4111. These conductive traces (not shown in the figure) are electrically connected to multiple golden fingers 41130 of the wiring portion 4113 in one-to-one correspondence.

The electrical functional component 41 is adhered to the adhesive-backed layer 42 at the center through a biocompatible adhesive (not shown in the figure), and a threading hole 421 is arranged at a position of the adhesive-backed layer 42 corresponding to the end of the wiring portion 4113. The threading hole 421 can allow the end of the wire 44 to pass through and be electrically connected with the wiring portion 4113, so as to avoid affecting tight fitting of the insulated electrode 400 and the human skin when the wire 44 is pressed between the adhesive-backed layer 42 and the human skin, thereby further avoiding a low-temperature scald caused because more heat is generated by the electrical functional component 41 when impedance between the electrical functional component 41 and the skin is increased if the air enters a space between the electrical functional component 41 and the human skin.

The supporting member 43 has multiple through-holes 431, and the through-holes 431 correspond to the corresponding electrode elements 410. The supporting member 43 can be an integral sheet-shaped structure, which can improve the overall strength of the insulated electrode 400. The multiple through-holes 431 are arranged at intervals and are respectively arranged on the supporting member 43 in a manner that surrounding the corresponding electrode elements 410. In this embodiment, the supporting member 43 is jointly constituted by the multiple independent support units 430 with the same structure. The multiple support units 430 are arranged at intervals. Each support unit 430 surrounds peripheries of multiple corresponding electrode elements 410. Each support unit 430 has two through-holes 431 for respectively accommodating two adjacent electrode elements 410 in the same row. The supporting member 43 is formed by ten support units 430. A thickness of the supporting member 43 is basically consistent with a thickness of the electrode element 410. After the supporting member 43 and the electrical functional component 41 are adhered to the adhesive-backed layer 42, top surfaces of the supporting member 43 and the electrode elements 410 are basically flush. In other embodiments, each support unit 430 may be provided with a single through-hole 431 with a larger size to surround the periphery of the multiple electrode elements 410 in rows.

Each adhesive member 45 is applied to surfaces of the corresponding supporting member 43 and the electrode elements 410 that are away from the adhesive-backed layer 42. The adhesive member 45 has adhesiveness on both sides, and can keep skin surface moist and relieve local pressure when in contact with the skin. The adhesive member 45 is preferably conductive gel. The shape of the adhesive member 45 is substantially the same as the shape of the supporting member 43. Because the top surfaces of the supporting members 43 and the electrode elements 410 are flush, the adhesive member 45 is evenly covered on the corresponding supporting member 43 and the electrode elements 410.

The insulated electrode 400 applies the alternating electric signal to the tumor site of the patient through the at least 10 electrode elements 410 provided thereon for tumor treatment, which can prevent the therapeutic effect from being affected due to insufficient electric field treatment caused by differences in tumor sizes, sites, and positions, increase the coverage area of the electrode elements 410 of the insulated electrode 400, enhance the intensity of the electric field applied to the tumor site for the tumor-treating fields therapy, and increase a range of covering the tumor site by the alternating electric field, thereby improving the therapeutic effect.

The multiple adjacent electrode elements 410 are arranged at intervals, to form a plurality of open spaces 416 to allow the skin of the body surface corresponding to the tumor site of the patient that is covered by the insulated electrode 400 to breathe freely after the insulated electrode 400 is adhered to the body surface corresponding to the tumor site of the patient. Sizes of the electrode element 410, the connecting portion 412, and the wiring portion 413 of the electrical functional component 41 and sizes of the open spaces 416 are described in detail below.

The electrode element 410 substantially has a circular sheet-shaped structure and a diameter of 21 mm to 22 mm. Distances between a plurality of two adjacent electrode elements 410 arranged in rows are the same. Distances between a plurality of two adjacent electrode elements 410 in the same row are at least 8 mm. Distances between a plurality of two adjacent electrode elements 410 arranged in columns are the same. Distances between a plurality of two adjacent electrode elements 410 in the same column are at least 6.5 mm. Distances between a plurality of two adjacent electrode elements 410 diagonally arranged in adjacent rows and adjacent columns are the same. Distances between a plurality of two adjacent electrode elements 410 arranged diagonally in adjacent rows and adjacent columns are about at least 19 mm.

The electrical functional component 41 is substantially arranged in a shape of an octagonal sheet, and has a minimum length of 166 mm and a minimum width of 103.5 mm. That is, all electrode elements 410 of the electrical functional component 41 are spaced apart within a minimum region of 166 mm×103.5 mm. The insulated electrode with such size can be used by children with small waist circumferences.

In order to avoid affecting the therapeutic effect due to overlapping of electrical functional components 41 after the insulated electrode 400 is attached to the body surface corresponding to the tumor site of the patient, the maximum distance between the two adjacent electrode elements 410 in the same row is 22 mm, the maximum distance between the two adjacent electrode elements 410 in the same column is 25 mm, and the distance between the two adjacent electrode elements 410 diagonally arranged in adjacent rows and adjacent columns is at least approximately 33.3 mm. The maximum diameter of the electrode element 410 is 22 mm, and the electrical functional component 41 has the maximum length of 242 mm and the maximum width of 166 mm. That is, all electrode elements 410 of the electrical functional component 41 are spaced apart within a maximum region of 242 mm×166 mm. The maximum size of the aforementioned electrical functional component 41 is suitable for most adult patients. If a waist of the patient is too large, two pairs of the insulated electrodes 400 can be applied transversely at the perimeter of the waist of the patient. If a waist of the patient is too small, two pairs of the insulated electrodes 400 can be applied longitudinally at the perimeter of the waist of the patient. If the patient has an intermediate waist, one pair of the insulated electrodes 400 can be applied transversely to the waist of the patient, another pair of the insulated electrodes 400 can be applied longitudinally to the waist of the patient, and the two pairs of the insulated electrodes 400 are applied at the perimeter of the waist of the patient.

Lengths of multiple connecting portions 4112 each connecting two adjacent electrode elements 410 in the same row are 8 mm to 22 mm, and lengths of multiple connecting portions 4112 each connecting two adjacent electrode elements 410 in the same column are 6.5 mm to 25 mm. Lengths of multiple connecting portions 4112 each connecting two adjacent electrode elements 410 diagonally arranged in adjacent rows and adjacent columns are approximately 19 mm to 33.3 mm. Widths of all connecting portions 4112 between two adjacent electrode elements 410 are 4.5 mm to 6 mm. Preferably, the width of the connecting portion 4112 is 4.5 mm.

The wiring portion 4113 is located between the multiple electrode elements 410 and is arranged in the open space 416 surrounded by the multiple electrode elements 410, which can avoid an increase in manufacturing costs due to an excessively large overall size of the electrical functional component 41. The wiring section 4113 includes a bridge section 4113A bridging two opposite connecting portions 4112 and a wiring section 4113B connected to the wire 44. The wiring section 4113B and the bridge section 4113A are arranged perpendicularly. The bridge section 4113A of the wiring portion 4113 and the two connecting portions 4112 connected to the bridge section 4113A are both arranged perpendicularly, and the wiring section 4113B of the wiring section 4113 and the two connecting portions 4112 connected to the wiring portion 4113 are arranged in parallel. The bridge section 4113A of the wiring section 4113 bridges two adjacent connecting sections 4112 in the middle, and the wiring section 4113B of the wiring portion 4113 is arranged by laterally extending the middle part of the bridge section 4113A of the wiring portion 4113. The width of the wiring section 4113B of the wiring portion 4113 is at least 4 mm, and a distance between the wiring section 4113B of the wiring portion 4113 and the electrode element 410 adjacent to the wiring section 4113B is at least 2 mm. Preferably, the width of the wiring section 4113B of the wiring portion 4113 is 4 mm to 8 mm. The width of the bridge section 4113A of the wiring portion 4113 is 4.5 mm to 6 mm, and a distance between the bridge section 4113A of the wiring portion 4113 and the electrode element 410 adjacent to the bridge section 4113A is at least 1 mm. Preferably, the width of the bridge section 4113A of the wiring portion 4113 is the same as the width of the connecting portion 4112.

Preferably, in this embodiment, referring to FIG. 20 and FIG. 21, a diameter of the electrode element 410 is 21 mm. A distance between two adjacent electrode elements 410 arranged in rows is 15 mm. A distance between two adjacent electrode elements 410 arranged in columns is 23 mm. Lengths of multiple connecting portions 4112 each connecting two adjacent electrode elements 410 in the same row are 15 mm, lengths of multiple connecting portions 4112 each connecting two adjacent electrode elements 410 in the same column are 23 mm, and lengths of multiple connecting portions 4112 each connecting two adjacent electrode elements 410 diagonally arranged in adjacent rows and adjacent columns are approximately 27.5 mm. A width of the connecting portion 4112 is 4.5 mm. The width of the bridge section 4113A of the wiring portion 4113 is 4.5 mm, the width of the wiring section 4113B of the wiring portion 4113 is 8 mm, and the length of the wiring section 4113B of the wiring portion 4113 is 42 mm. The electrical functional component 41 has a length of 201 mm and a width of 153 mm. Areas of the open spaces 416 of the electrical functional component 41 that are formed between the multiple electrode elements 410 are not completely the same. In this embodiment, in all the open spaces 416, an open space 416 that is located between eight electrode elements 410 in the middle two columns and that allows the wiring section 4113 to pass through has the largest area of about 4428 mm². The open spaces 416 located at the four corners of the electrical functional component 41 have the smallest area. The open space 416 located at the corner of the electrical functional component 41 is jointly enclosed by three electrode elements 410 that are adjacent to each other in pairs in the same row and column and that are in adjacent rows and adjacent columns in pairs diagonally, and has an area of about 452 mm. An area of the open spaces 416 enclosed by other four electrode elements 410 located in adjacent rows and adjacent columns is approximately 1066 mm².

The insulated electrode 400 in this embodiment has a plurality of open spaces 416 located between multiple electrode elements 410, and in the process of the tumor-treating fields therapy, heat accumulated on the corresponding skin surface of the patient where the insulated electrode 400 is applied and the water vapor generated during sweating can be discharged into the external air through the open spaces 416, so as to avoid skin symptoms such as erythema, itching and hair follicle inflammations of skin, pains, and pimples.

### A Fifth Embodiment of the Insulated Electrode

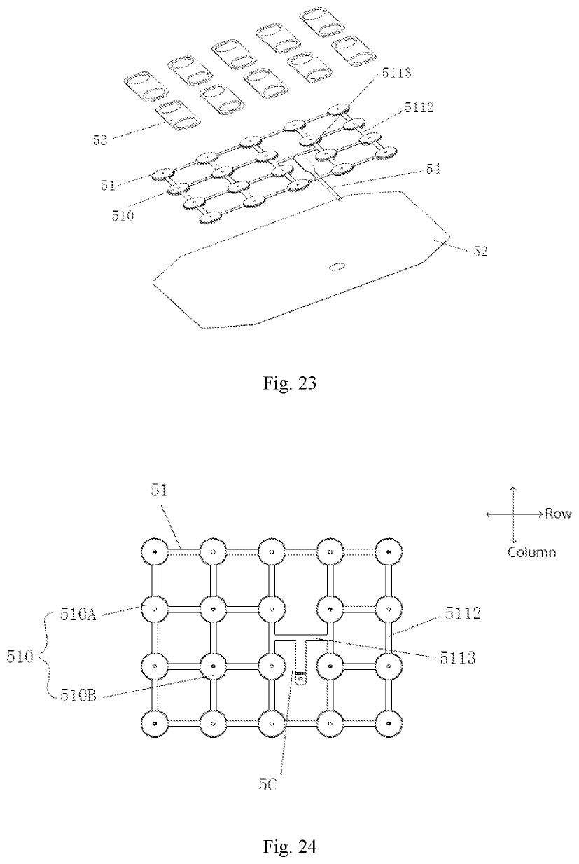

Referring to FIG. 22 to FIG. 24, an insulated electrode 500 in this embodiment includes an adhesive-backed layer 52, an electrical functional component 51 adhered to the adhesive-backed layer 52, a plurality of supporting members 53 adhered to the adhesive-backed layer 52, a plurality of adhesive members (not shown in the figure) each covers corresponding parts of the corresponding supporting member 53 and the electrical functional component 51, and a wire 54 electrically connected to the electrical functional component 51.

The insulated electrode 500 in this embodiment is basically the same as the insulated electrode 400 in the fourth embodiment, and an only difference is that a specific arrangement of the electrode elements 510 of the electrical functional component 51 is different. Only the difference is described below. For other content, refer to the fourth embodiment.

The electrical functional component 51 is arranged in a shape of a grid, and includes multiple electrode elements 510 arranged in a rectangular array, multiple connecting portions 5112 each connected to two adjacent electrode elements 510, and a wiring portion 5113 electrically connected to a wire 54. Each electrode element 510 is connected with at least two electrode elements 510 adjacent thereto via corresponding connecting portions 5112. Each electrode element 510 is connected with at least two connecting portions 5112. The multiple electrode elements 510 are distributed at intervals at grid points of the electrical functional component 51. The multiple electrode elements 510 are distributed in a region surrounded by an array with at least three rows and four columns, and there are at least twelve electrode elements 510 and at most 30 electrode elements 510, which can increase a coverage area of the electrode elements 510 of the insulated electrode 500, enhance the intensity of the electric field applied to the tumor site for tumor-treating fields therapy, and increase a range of covering the tumor site by the alternating electric field, thereby improving a therapeutic effect. Multiple electrode elements 510 are distributed in an array region with three rows and four columns, and the number of the electrode elements is 12; or the multiple electrode elements are distributed in an array region with three rows and five columns, the number of the electrode elements is at least 12 and at most 15; or the multiple electrode elements are distributed in an array region with four rows and four columns, the number of the electrode elements is at least 12 and at most 16; or the multiple electrode elements are distributed in an array region with four rows and five columns, the number of the electrode elements is at least 12 and at most 20; or the multiple electrode elements are distributed in an array region with four rows and six columns, the number of the electrode elements is at least 12 and at most 24; or the multiple electrode elements are distributed in an array region with five rows and five columns, the number of the electrode elements is at least 12 and at most 25; or the multiple electrode elements are distributed in an array region with five rows and six columns, the number of the electrode elements is at least 12 and at most 30.

The numbers of electrode elements 510 located in all rows are the same and the electrode elements are aligned in a column direction. The numbers of electrode elements 510 located in all columns are the same and the electrode elements are aligned in a row direction. Distances between a plurality of two adjacent electrode elements 510 arranged in rows are the same, and distances between a plurality of two adjacent electrode elements 510 arranged in columns are also the same. Two adjacent electrode elements 510 in the same row are arranged in adjacent columns, and two adjacent electrode elements 510 in the same column are arranged in adjacent rows. The connecting portions 5112 are located between two adjacent electrode elements 510 in the same row or in the same column. Multiple connecting portions 5112 each connecting two adjacent electrode elements 510 arranged in rows have the same length. Multiple connecting portions 5112 each connecting two adjacent electrode elements 510 arranged in columns have the same length. A distance between two adjacent electrode elements 510 arranged in rows is different with a distance between two adjacent electrode elements 510 arranged in columns. That is, a length of a connecting portion 5112 between two adjacent electrode elements 510 arranged in rows is different with a length of a connecting portion 5112 between two adjacent electrode elements 510 arranged in columns. Optionally, a distance between two adjacent electrode elements 510 arranged in rows is same as a distance between two adjacent electrode elements 510 arranged in columns. That is, a length of a connecting portion 5112 between two adjacent electrode elements 510 arranged in rows is same as a length of a connecting portion 5112 between two adjacent electrode elements 510 arranged in columns.

At least one pair of two adjacent electrode elements 510 in the multiple electrode elements 510 are arranged in a disconnected manner. A gap 5C allowing the wiring portion 5113 to pass through is formed between the two adjacent electrode elements 510 arranged in the disconnected manner. The wiring portion 5113 may be arranged in a shape of Chinese character “−” and is arranged by laterally extending from the connecting portion 5112 opposite to the gap 5C, or the wiring portion 5113 may also be arranged in a shape of “T” and is provided between the two connecting portions 5112 that are respectively connected to the two electrode elements 510 arranged in the disconnected manner. The electrode elements 510 arranged in the disconnected manner are located at the inner of the array region where the electrode elements 510 are located. The electrode elements 510 of the electrical functional component 51 located at the periphery or the array are all connected with each other through the corresponding connecting portions 5112. That is, all the plurality of two adjacent electrode elements 510 located at the periphery of the electrical functional component 51 are all connected with each other through the connecting portions 5112. At least one pair of adjacent electrode elements 510 diagonally arranged in adjacent rows and adjacent columns in the multiple electrode elements 510 are arranged in a disconnected manner. The wiring portion 5113 is located between the multiple electrode elements 510, which can avoid an increase in manufacturing costs due to an excessively large overall size of the electrical functional component 51.

From the perspective of distribution positions of the electrode elements 510 in the array, the multiple electrode elements 510 can be divided into multiple peripheral electrode elements 510A located at the periphery and multiple central electrode elements 510B surrounded by the peripheral electrode elements 510A. There are at least ten peripheral electrode elements 510A, and there are at least two central electrode elements 510B. All the peripheral electrode elements 510A are connected with each other through the corresponding connecting portions 5112. That is, the connecting portions 5112 are arranged between all pairs of adjacent peripheral electrode elements 510A. At least one central electrode element 510B is arranged in a disconnected manner with a peripheral or central electrode element 510A, 510B located in the same row or in the same column and adjacent thereto, and a gap 5C is formed between the two adjacent electrode elements arranged in a disconnected manner to allow the wiring portion 5113 to pass through.

The wiring portion 5113 can be arranged by laterally extending the connecting portion 5112 opposite to the gap 5C toward the gap 5C, and has a structure substantially in a shape of Chinese character “−”. The connecting portion 5112 laterally extending to provide the wiring portion 5113 is arranged perpendicular to the wiring portion 5113, and is substantially arranged in a shape of “T” with the wiring portion 5113. The connecting portion 5112 laterally extending to form the wiring portion 5113 is located between two adjacent peripheral electrode elements 510A, or may be located between a peripheral electrode element 510A and an adjacent central electrode element 510B, or between two adjacent central electrode elements 510B. That is, the connecting portion 5112 laterally extending to provide the wiring portion 5113 connects two adjacent peripheral electrode elements 510A, or connects two adjacent central electrode elements 510B, or connects one peripheral electrode element 510A to an adjacent central electrode element 510B. The wiring portion 5113 may also be arranged in a shape of “T” and bridges two connecting portions 5112 respectively connected with two central electrode elements 510B arranged in the disconnected manner, or bridges two connecting portions 5112 respectively connected with one central electrode element 510B and one peripheral electrode element 510A that are adjacent to each other and arranged in the disconnected manner.

In other embodiments, at least one pair of adjacent peripheral electrode elements 510A in the multiple peripheral electrode elements 510A are disconnected, and at least one of the peripheral electrode elements 510A arranged in a disconnected manner is connected through a connecting portion 5112 to a central electrode element 510B arranged diagonally in adjacent rows and adjacent columns therewith. That is, some pairs of adjacent peripheral electrode elements 510 are connected through the corresponding connecting portions 5112. Some pairs of adjacent peripheral electrode elements 510A are disconnected, and no connecting portion 5112 is arranged therebetween. The connecting portions 5112 are arranged between one peripheral electrode element 510A and one central electrode element 510B that are arranged diagonally in adjacent rows and adjacent columns, between two adjacent peripheral electrode elements 510A, between two adjacent central electrode elements 510B, and between one peripheral electrode element 510A and one adjacent central electrode element 510B that are arranged in the same row or in the same column.

In this embodiment, the multiple electrode elements 510 of the electrical functional component 51 are arranged in four rows and five columns. The number of the electrode elements 510 of the electrical functional component 51 is twenty. The numbers of electrode elements 510 in all rows are the same and the numbers of electrode elements 510 in all columns are also the same. The number of the electrode elements 510 in each row is 5. The number of the electrode elements 510 in each column is 4. The electrode element 510 located in the third column of the second row and the electrode element 510 located in the fourth column of the second row are arranged in the disconnected manner, and a gap 5C is formed therebetween. The electrode element 510 in the third column of the third row and the electrode element 510 in the fourth column of the third row are arranged in the disconnected manner, and a gap 5C is also formed therebetween. The wiring portion 5113 is arranged in a shape of “T”, and is provided between the connecting portion 5112 in the middle of the third column and the connecting portion 5112 in the middle of the fourth column. The connecting portion 5112 in the middle of the third column is provided between two electrode elements 510 respectively located in the second row of the third column and in the fourth row of the third column. The connecting portion 5112 in the middle of the fourth column is provided between two electrode elements 510 respectively located in the second row of the fourth column and in the third row of the fourth column. The connecting portions 5112 are respectively located between all pairs of adjacent electrode elements 510 in the same row or in the same column except the two electrode elements 510 respectively located in the third column of the second row and the fourth column of the second row, and the two electrode elements 510 respectively located in the third column of the third row and the fourth column of the third row.

The insulated electrode 500 in this embodiment applies the alternating electric field to the tumor site of the patient through the multiple electrode elements 510 provided thereon for the electric field treatment, which can prevent the therapeutic effect from being affected due to insufficient intensity of the alternating electric field applied to the tumor site for the electric field treatment caused by differences in tumor sizes, sites, and positions, increase the coverage area of the electrode element 510 of the insulated electrode 500, enhance the intensity of the electric field applied to the tumor site for the tumor-treating fields therapy, and increase a range of covering the tumor site by the alternating electric field, thereby improving the therapeutic effect.

### A Sixth Embodiment of the Insulated Electrode

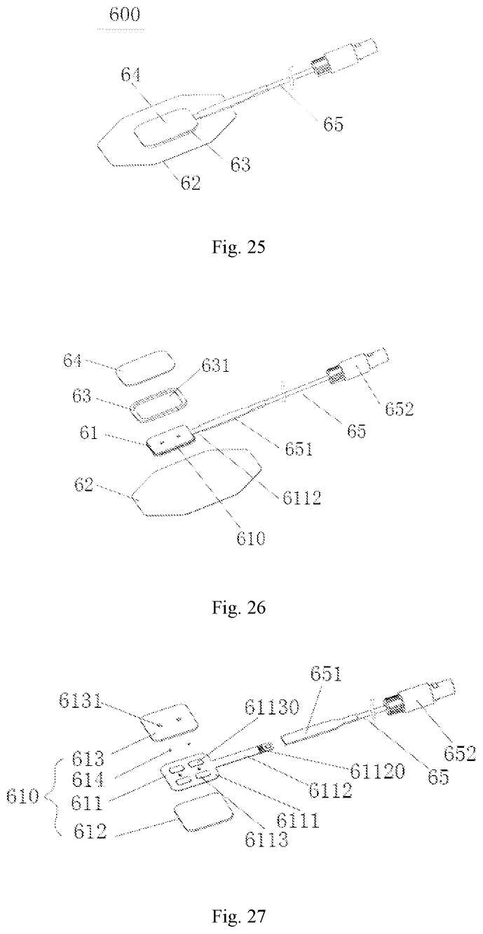

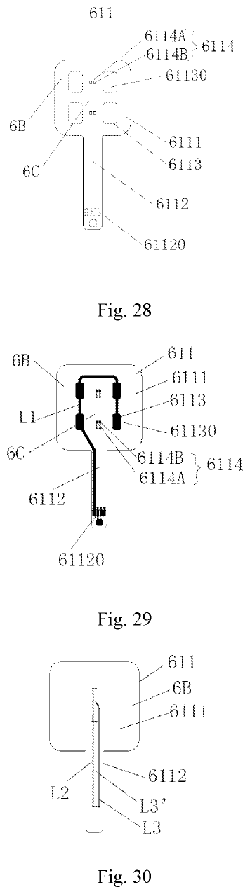

The electrical functional components of the insulated electrodes in the preceding embodiments are provided with multiple electrode elements, and the multiple electrode elements are arranged in series. If one of electrode elements is damaged, the entire insulated electrode is scrapped, and the costs of scrapping are higher. Therefore, this embodiment provides another form of insulated electrode. FIG. 25 to FIG. 30 show an insulated electrode 600 in the sixth embodiment of the present invention. Multiple the insulated electrodes 600 in this embodiment can be combined for use, and the multiple the insulated electrodes 600 are connected to a hub (not shown in the figure) to jointly carry out the tumor-treating fields therapy for the tumor site. The insulated electrode 600 includes an adhesive-backed layer 62, an electrical functional component 61 adhered to the adhesive-backed layer 62, a supporting member 63 adhered to the adhesive-backed layer 62, an adhesive member 64 that covers corresponding parts of the supporting member 63 and the electrical functional component 61 and that fits skin on the body surface corresponding to the tumor site of the patient, and a wire 65 electrically connected to the electrical functional component 61. The insulated electrode 600 is attached to the body surface corresponding to the tumor site of the patient through the adhesive-backed layer 62, and applies an alternating electric field to the tumor site of the patient through the electrical functional component 61 to interfere with or prevent mitosis of cancer cells of the patient, thereby achieving a tumor treatment purpose.

The electrical functional component 61 includes a single electrode element 610 arranged in a shape of a square sheet and a wiring portion 6112 connected to the electrode element 610. The wiring portion 6112 is welded to the wire 65 to achieve the electrical connection between the electrical functional component 61 and the wire 65. Multiple golden fingers 61120 are provided on one side surface of the wiring portion 6112. In this embodiment, the number of golden fingers 61120 is four, and the four golden fingers 61120 are arranged on one side surface of the wiring portion 6112 facing toward the skin. The welding joints between the wire 65 and the golden fingers 61120 of the wiring portion 6112 are externally covered with a heat shrinkable sleeve 651. The heat shrinkable sleeve 651 insulates and protects the joints between the wire 65 and the wiring portion 6112 of the electrical functional component 61 and provides support, thereby avoiding breakage of the joints between the wire 65 and the wiring portion 6112 of the electrical functional component 61 and further achieving dust-proof and waterproof purposes. An end of the wire 65 away from the wiring portion 6112 is provided with a plug 652 electrically connected to the electric field generator (not shown in the figure) or a hub (not shown in the figure). The wire 65 has one end electrically connected to the golden fingers 61120 of the wiring portion 6112, and the other end electrically connected to an electric field generator (not shown in the figure) or a hub (not shown in the figure) through the plug 652 to provide the insulated electrode 600 with an alternating electric signal for tumor treatment during tumor-treating fields therapy.

The electrode element 610 includes a main body 6111, an insulating plate 612 arranged on a side of the main body 6111 away from human skin, a dielectric element 613 arranged on a side of the main body 6111 facing toward the human skin, and two temperature sensors 614 arranged on the main body 6111 and located on the same side as the dielectric element 613. The main body 6111, the insulating plate 612, and the dielectric element 613 have approximately the same shapes, and are all square sheet-shaped structures. The main body 6111, the insulating plate 612 and the dielectric element 613 are correspondingly arranged along a thickness direction of the main body 6111, and have centers located on the same straight line. In this embodiment, the main body 6111, the insulating plate 612, and the dielectric element 613 are all square sheet-shaped structures with arc corners. Preferably, the main body 6111 is a square sheet-shaped structure with a size of about 32 mm×32 mm. The wiring portion 6112 of the electrical functional component 61 is arranged by laterally extending the main body 6111 of the electrode element 610.

The main body 6111 includes an insulating substrate 6B and four conductive traces L embedded in the insulating substrate 6B. The four conductive traces are respectively the first conductive trace L1 arranged on one side of the insulating substrate 6B closer to the dielectric element 613, the second conductive trace L2 arranged on one side of the insulating substrate 6B closer to the insulating plate 612 and two third conductive traces L3 and L3′ on the same side as the second conductive trace L2. The main body 6111 is provided with a conductive pad 6113 exposed from the middle of the insulating substrate 6B thereof and electrically connected with the first conductive trace L1. The conductive pad 6113 can be welded with the dielectric elements 613 to mount the dielectric elements 613 onto the corresponding main body 6111. The conductive pad 6113 can be completely covered by the dielectric element 613, so that the conductive pad 6113 and the dielectric element 613 can be welded through soldering tin (not shown in the figure). The center of the conductive pad 6113 is located on a center line of the main body 6111. The conductive pad 6113 includes multiple conductive cores 61130 center-symmetrically arranged, which can effectively prevent the position of the dielectric element 613 from being shifted due to accumulation of soldering tin (not shown includes in the figure) during the welding process. Top surfaces of multiple conductive cores 61130 are located in the same plane, so that virtual welding can be avoided during the welding process of the dielectric element 613. The multiple conductive cores 61130 are all connected to the first conductive trace L1. The multiple conductive cores 61130 are connected in series by the first conductive trace L1.

In this embodiment, the conductive pad 6113 of the main body 6111 has a substantially square structure, and has symmetry axes overlapped with symmetry axes of the main body 111. The conductive pad 6113 includes 4 conductive cores 61130 located at four corners thereof and arranged at intervals. The conductive cores 61130 are arranged in a multi-point interval mode, which can reduce the consumption of copper foil used to manufacture the conductive cores 61130. In addition, this can also reduce the amount of soldering tin (not shown in the figure) used to weld the conductive cores 61130 with the dielectric element 613, and reduce manufacturing costs. Each conductive core 61130 has a rectangular structure with a size of approximately 9 mm×6 mm. Preferably, each conductive core 61130 has a rectangular structure with four rounded corners. A longitudinal long axis of each conductive core 61130 is parallel to an extension direction of the wiring portion 6112. In other embodiments, each conductive core 61130 of the conductive pad 6113 can also be round, square, or the like.

In this embodiment, the four conductive cores 61130 forming the conductive pad 6113 are arranged into an array, and the four conductive cores 61130 are arranged in two rows and two columns. A gap between the two columns of conductive cores 61130 is about 8.5 mm, and a gap between the two rows of conductive cores 61130 is about 4 mm. The four conductive cores 61130 constituting the conductive pad 6113 are both center-symmetrically and axial-symmetrically arranged, and each conductive core 61130 is also axial-symmetrically arranged. Therefore, when the four conductive cores 61130 of the main body 6111 are welded with the dielectric element 613, stress balance between welding points is ensured, overall welding balance of the dielectric element 613 is ensured, and welding quality is improved, which prevents a welding joint on the side of the dielectric element 613 at a larger distance from the main body part 6111 being weak and prone to break because the dielectric element 613 is tilted when the welding stress is unbalanced, and can also avoid affecting fitting of the insulated electrode 600. The four conductive cores 61130 of the conductive pad 6113 are arranged at intervals in pairs, and a gap 6C is formed between the two adjacent conductive cores 61130. Four gaps 6C are substantially connected with each other in a shape of “+”. Adjacent gaps 6C are connected with each other. In the four gaps 6C, an extension direction of two gaps 6C between the two conductive cores 61130 in the same row is consistent with an extension direction of the wiring portion 6112.

The main body 6111 has two pairs of pads 6114 exposed from the insulating substrates 6B thereof and welded to corresponding parts of the corresponding temperature sensor 614 to achieve the electrical connection with the temperature sensor 614. Each pair of pads 6114 are located between the two corresponding conductive cores 61130 arranged in the same row at intervals. The two pairs of pads 6114 are both arranged in the extension direction of the wiring portion 6112, and each pair of pads 6114 has one symmetrical center. A connecting line of the two symmetrical centers of the two pairs of pads 6114 is parallel to the extension direction of the wiring portion 6112. Each pair of pads 6114 include a first pad 6114A and a second pad 6114B. The first pad 6114A in each pair of pads 6114 is electrically connected to the second conductive trace L2. One of the two second pads 6114B is electrically connected to the third conductive trace L3, and the other one of the two second pads 6114B is electrically connected to the third conductive trace L3′. Each temperature sensor 614 has a signal end (not shown in the figure) and a ground end (not shown in the figure). The first pad 6114A is welded to the ground end (not shown in the figure) of the temperature sensor 614, and the second pad 6114B is welded to the signal end (not shown in the figure) of the corresponding temperature sensor 614.

The insulating plate 612 is made of an insulated material. Preferably, the insulating plate 612 is an epoxy glass cloth laminate plate. The insulating plate 612 is adhered to a surface of the main body 6111 away from human skin through the sealant (not shown in the figure), which provide a flat welding plane for a welding operation between the main body 6111 and the dielectric element 613 while enhancing the strength of the main body 6111, thereby improving product yield rate. In addition, the insulating plate 612 can further prevent water vapor in the air on the side of the insulated electrode 600 away from the skin coming into contact with the soldering tin (not shown in the figure) between the main body 6111 and the dielectric element 613, and further prevent an electrical connection between the main body 6111 and the dielectric element 613 being affected because the water vapor corrodes the soldering tin (not shown in the figure) located between the main body 6111 and the dielectric element 613.

The size of the insulating plate 612 is the same as that of the main body 6111, so as to prevent the sealant (not shown in the figure) from climbing to the side of the main body part 6111 facing the human skin by capillary effect when the insulating plate 612 is adhered to a surface of the main body part 6111 away from human skin by sealant (not shown in the figure), which may affect the filling of the sealant (not shown in the figure) in a gap (not shown in the figure) formed by welding the dielectric element 613 and the main body part 6111, resulting the existence of cavities in the sealant (not shown in the figure). This may further prevent the sealant (not shown in the figure) from bursting due to the rapid expansion of water vapor caused by a large difference in thermal expansion coefficient between the sealant (not shown in the figure) and the water vapor in the cavities during high-temperature curing, which may lead to popcorn phenomenon, and damage the products.

The dielectric element 613 is made of a material with a high dielectric constant, which has a property of impeding conduction of the direct current and allowing conduction of the alternating current, thereby ensuring safety of human body. Preferably, the dielectric element 613 is a dielectric ceramic plate. The dielectric element 613 is provided with two through-holes 6131 the number of which is the same as the number of temperature sensors 614, and the two through-holes 6131 are used to accommodate the corresponding temperature sensors 614. A metal layer (not shown in the figure) is attached to the surface of the dielectric element 613 facing toward the main body 6111. A Point-to-side welding is formed between the metal layer (not shown in the figure) of the dielectric element 613 and the conductive cores 61130 of the conductive pad 6113 of the main body 6111, without requiring high welding alignment accuracy, thereby facilitating welding. The inner edge of the metal layer (not shown in the figure) of the dielectric element 613 and the through-hole 6131 of the dielectric element 613 are arranged at intervals, which can avoid a short circuit of the temperature sensor 614 that is caused because the soldering tin (not shown in the figure) between the metal layer (not shown in the figure) of the dielectric element 613 and the main body 6111 spreads toward the through-hole 6131 of the dielectric element 613 when heated and melted. The outer edge of the metal layer (not shown in the figure) of the dielectric element 613 and the outer edge of the dielectric element 613 are also arranged at intervals, which can prevent the soldering tin (not shown in the figure) between the metal layer (not shown in the figure) of the dielectric element 613 and the main body 6111 from melting and overflowing to the outer side of the main body 6111 when heated, and prevent the direct current unimpeded by the dielectric element 613 from passing through and directly acting on the body surface of the patient when the insulated electrode 600 is attached to the body surface corresponding to the tumor site of the patient.

The gap (not shown in the figure) formed by welding the dielectric element 613 and the main body 6111 is filled with the sealant (not shown in the figure) to protect the soldering tin (not shown in the figure) between the dielectric element 613 and the main body 6111, thereby preventing the alternating electric field being unable to be applied to the tumor site of the patient through the dielectric element 613 because the welding joint is broken when the dielectric element 613 is affected by external force, and further preventing an electrical connection between the dielectric element 613 and the main body 6111 being affected because the water vapor in the air enters the gap (not shown in the figure) and corrodes the soldering tin (not shown in the figure) between the dielectric element 613 and the main body 111. The size of the dielectric element 613 is slightly less than the size of the main body 6111, and the sealant (not shown in the figure) is filled inside the gap (not shown in the figure) along the edge of the main body 6111 outside of the dielectric element 613 through a capillary phenomenon when the sealant (not shown in the figure) is filled, so as to facilitate the filling of the sealant (not shown in the figure) into the gap (not shown in the figure) formed by the welding the dielectric element 613 to the main body 6111. When the gap (not shown in the figure) formed by welding the dielectric element 613 to the main body 6111 is filled with the sealant (not shown in the figure), the air in the gap (not shown in the figure) can be discharged through the through-hole 6131 of the dielectric element 613, so as to avoid the voids generated in the sealant (not shown in the figure) filled in the gap (not shown in the figure), thereby improving quality of the product.

Each temperature sensor 614 achieves the electrical connection with the main body 6111 through welding the ground end (not shown in the figure) thereof with the first pad 6114A located on the main body 6111 and the signal end (not shown in the figure) thereof with one second pad 6114B located on the main body 6111. Two first pads 6114A of the main body 6111 are electrically connected to the second conductive trace L2, one of the two second pads 6114B is electrically connected to the third conductive trace L3, the other one of the two second pads 6114B is electrically connected to the third conductive trace L3′, the two first pads 6114A are respectively welded to corresponding ground ends (not shown in the figure) of the two temperature sensors 614, and the two second pads 6114B are respectively welded to corresponding signal ends (not shown in the figure) of the two temperature sensors 614. Therefore, the ground ends (not shown in the figure) of the two temperature sensors 614 are both electrically connected to the second conductive trace L2 of the main body 6111, and signal ends of the two temperature sensors 614 (not shown in the figure) are respectively and electrically connected to the third conductive traces L3 and L3′ of the main body 6111. That is, the temperature signals monitored by the two temperature sensors 6114 are transmitted through the second conductive trace L2 and the third conductive traces L3 and L3′.

After being welded to the main body 6111, the two temperature sensors 614 are respectively accommodated in the corresponding through-holes 6131 of the dielectric element 613. Preferably, the temperature sensor 614 is a thermistor. Each temperature sensor 614 is configured to monitor the temperature of the adhesive member 64 covering one surface of the dielectric element 613 of the electrical functional component 61 facing toward the human skin, to further detect temperature of the human skin to which the adhesive member 64 is attached. When the temperature monitored by the temperature sensor 614 exceeds an upper safe temperature limit of the human body, the tumor electric field therapy system (not shown in the figure) can promptly reduce the alternating voltage or alternating current of the alternating electrical signal or cut off the alternating electrical signal applied to the insulated electrode 600 to avoid a low-temperature burn to the human body. The two temperature sensors 614 are symmetrically arranged on the main body 6111, which can detect temperature of corresponding human skin at different positions, thereby ensuring reliability of detected data. Two temperature sensors 614 are welded to the main body 6111 through two pairs of pads 6114 of the main body 6111 and then sealed with the sealant (not shown in the figure) to prevent water vapor from corroding the temperature sensors 614 and causing the temperature sensors 614 to fail.

The wiring portion 6112 has the same components as the main body portion 6111, and also has a corresponding insulating substrate 6B and four conductive traces L embedded in the insulating substrate 6B. The four conductive traces L of the wiring portion 6112 are also electrically connected to the corresponding conductive traces L of the main body 6111, respectively. The four golden fingers 61120 of the wiring portion 6112 are exposed from one side surface of the insulating substrate 6B closer to the dielectric element 613. The four conductive traces L of the wiring portion 6112 are electrically connected to the golden fingers 61120, respectively. The four conductive traces L of the wiring portion 6112 are the first conductive trace L1, the second conductive trace L2 and the third conductive traces L3 and L3′, respectively. The first conductive trace L1 of the wiring portion 6112 is arranged by extending the first conductive trace L1 of the main body 6111. The second conductive trace L2 of the wiring portion 6112 is arranged by extending the second conductive trace L2 of the main body 6111. The third conductive traces L3 and L3′ of the wiring portion 113 are respectively arranged by extending the third conductive traces L3 and L3′ of the main body 6111.

The wiring portion 6112 achieves the electrical connection with the conductive pad 6113 of the main body 6111 through connecting the first conductive trace L1 thereof to the first conductive trace L1 of the main body 6111 and connecting the first conductive trace L1 of the main body 6111 to the conductive pad 6113 of the main body 6111, and then achieve the electrical connection with the dielectric element 613 through welding the conductive pad 6113 of the main body 6111 with the dielectric element 613. The wiring portion 6112 achieves the electrical connection with the first pad 6114A on the main body 6111 through connecting the second conductive trace L2 thereof to the second conductive trace L2 of the main body 6111 and connecting the second conductive trace L2 of the main body 6111 to the first pad 6114A of the main body 6111, and further achieves the electrical connection with the ground end (not shown in the figure) of the temperature sensor 614 through welding the first pad 6114A to the ground end (not shown in the figure) of the temperature sensor 614. The wiring portion 6112 achieves the electrical connection with two second pads 6114B on the main body 6111 through respectively connecting two third conductive traces L3, L3′ thereof to two third conductive traces L3, L3′ of the main body 6111, and respectively connecting two third conductive traces L3, L3′ of the main body 6111 to two second pads 6114B located on the main body 6111, and further achieves the electrical connection with signal ends of two temperature sensors 614 through welding two second pads 614B to the signal ends of two temperature sensor 614 in one-to-one correspondence, so as to transmit in parallel temperature signals monitored by the temperature sensors 614 to the electric field generator (not shown in the figure), so that the electric field generator (not shown in the figure) can adjust the alternating voltage or alternating current of the alternating electrical signal applied to the dielectric element 613 in time, thereby achieving a purpose of avoiding low-temperature scald caused by excessively high temperature.

The main body 6111 and the wiring portion 6112 jointly constitute the flexible circuit board 611 of the electrical functional component 61. The insulating substrates 6B of the main body 6111 and the wiring portion 6112 jointly form the insulating substrate 6B of the flexible circuit board 611. The conductive traces L of the main body 6111 and the conductive traces L of the wiring portion 6112 are in one-to-one correspondence to form the conductive traces L of the flexible circuit board 611. The insulating substrate 6B of the flexible circuit board 611 can isolate water vapor in the air around the insulated electrode 600 from the soldering tin (not shown in the figure) between the conductive pad 6113 and the dielectric element 613, and prevent water vapor in the air on the side away from the skin corroding the soldering tin (not shown in the figure) between the conductive pad 6113 on the main body 6111 of the flexible circuit board 611 and the dielectric element 613. The insulating substrate 6B of the flexible circuit board 611 and the insulating plate 612 have a function of double isolation, which can prolong service life of the insulated electrode 600.

From the perspective of formation of the electrode element 610, the insulating plate 612 is arranged on the side of the main body 6111 of the flexible circuit board 611 that is away from the human skin, the dielectric element 613 is arranged on the side of the main body 6111 of the flexible circuit board 611 facing toward the human skin, and the two temperature sensors 614 are arranged on the side of the main body 6111 of the flexible circuit board 611 facing toward the human skin. The insulating plate 612 and the dielectric element 613 are respectively arranged on two opposite sides of the main body 6111 of the flexible circuit board 611. The first conductive trace L1 of the flexible circuit board 611 connects the four spaced conductive cores 61130 of the conductive pad 6113 in series, the second conductive trace L2 is electrically connected to the ground ends (not shown in the figure) of the two temperature sensors 614 through two first pads 6114A, and the third conductive traces L3 and L3′ are electrically connected to the signal ends (not shown in the figure) of the two temperature sensors 614 through two second pads 6114B, respectively. The first conductive trace L1 is located at a layer within the insulating substrate 6B that is closer to human skin. The second conductive trace L2 and the third conductive traces L3 and L3′ are located at a layer within the insulating substrate 6B that is closer to the insulating plate 612. To facilitate arrangement of conductive traces L, a width of the wiring portion 6112 is 7 mm to 9 mm. Preferably, the width of the wiring portion 6112 is 8 mm.

The golden finger 61120 of the wiring portion 6112, the four conductive cores 61130 of the conductive pad 6113 and the pads 6114 are all exposed from one side surface of the insulating substrate 6B of the flexible circuit board 611 that is closer to the dielectric element 613. The golden fingers 61120, the four conductive cores 61130 of the conductive pad 6113 and the pads 6114 are all on one side of the flexible circuit board 611 that is closer to the body surface of the patient. One golden finger 61120 of the wiring portion 6112 has one end electrically connected to the dielectric element 613 through the first conductive trace L1 connected thereto and the other end welded to the corresponding part of the wire 65 to transmit the alternating electrical signal generated by the electric field generator (not shown in the figure) to the dielectric element 613. One golden finger 61120 of the other three golden fingers 61120 of the wiring portion 6112 has one end electrically connected to the ground end (not shown in the figure) of the temperature sensor 614 through the second conductive trace L2 connected thereto, and the other two golden fingers 61120 of the other three golden fingers 61120 have ends electrically connected to the signal ends (not shown in the figure) of two temperature sensors 614 through the third conductive traces L3 and L3′ connected thereto. The other three golden fingers 61120 of the wiring portion 6112 have other ends respectively welded to the corresponding parts of the wire 65, so that the relevant signals detected by the temperature sensors 614 are transmitted in parallel to the electric field generator (not shown in the figure) through the second conductive trace L2, the third conductive traces L3 and L3′, and the wire 65.

The adhesive-backed layer 62 is arranged in a shape of a sheet and is mainly made of a material with flexibility, breathability, and insulation. The adhesive-backed layer 62 is a mesh fabric. Specifically, the adhesive-backed layer 62 is a mesh-shaped nonwoven fabric, has characteristics of softness, lightness, moisture-proofing and breathability, and can still keep the skin surface of the patient dry when applied to the body surface of the patient for a long time. A surface of the adhesive-backed layer 62 facing toward the body surface of the patient is also coated with a biocompatible adhesive (not shown in the figure) for tightly fitting the adhesive-backed layer 62 to the corresponding body surface corresponding to the tumor site of the patient. In this embodiment, the adhesive-backed layer 62 is substantially arranged in a shape of an octagonal sheet.

The supporting member 63 is adhered to the adhesive-backed layer 62 and encloses the periphery of the electrode element 610. The supporting member 63 has a through-hole 631 in the middle thereof, to accommodate the electrode element 610. The supporting member 63 may be made of a foam material. The supporting member 63 is flush with a side surface of the electrode element 610 away from the adhesive-backed layer 62. That is, the supporting member 63 is flush with a side surface of the electrode element 610 facing toward the adhesive member 64, to support the adhesive member 64.

The adhesive member 64 has adhesiveness on both sides thereof. A surface of the adhesive member 64 is adhered to side surfaces of the supporting member 63 and the electrode element 610 that are away from the adhesive-backed layer 62. The other surface of the adhesive member 64 is used as an adhesion layer to be adhered to epidermis of the human, to keep the skin surface moist and relieve local pressure. Preferably, the adhesive member 64 is a conductive hydrogel to serve as a conductive medium. Under the support effect of the supporting member 63, the adhesive member 64 has better adhesion to human skin.

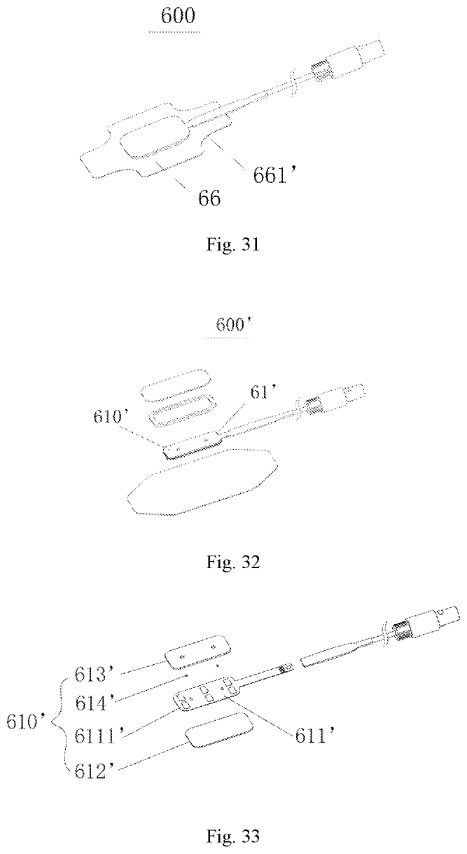

FIG. 31 shows another embodiment of an adhesive-backed layer of an insulated electrode 600. Four corners of the adhesive-backed layer 66 are recessed inward to form concave angles 661. The adhesive-backed layer 66 has a structure in approximately a shape of Chinese character “+”. The concave corner 661 communicates with the external environment and is arranged in a shape of “L”. When the insulated electrode 600 is adhered to the body surface corresponding to the tumor site of the patient, the concave angle 661 can avoid arching and folds at the corner of the adhesive-backed layer 66, to avoid a low-temperature scald caused because the electrical functional component 61 generates more heat when impedance between the electrical functional component 61 and the skin is increased if air enters space between the electrode element 610 and the skin from the folds.

Based on the insulated electrode 600 in this embodiment, because a single electrode element 610 is used to apply an alternating electrical signal to the tumor site of the patient, when the insulated electrode fails to operate normally, only the insulated electrode 600 with the single electrode element 610 needs to be replaced without a need to scrap the insulated electrodes 600 with multiple electrode elements 610 in the entire region, so that costs of the tumor treatment for the patient can be reduced. In addition, a random quantity of the insulated electrodes 600 in this embodiment can be combined based on the tumor site of the patient and the size of the tumor site of the patient, so as to ensure the coverage area of the insulated electrode 600 for the tumor-treating fields therapy and ensure the electric field treatment intensity. In addition, the relative positions of the multiple the insulated electrodes 600 can also be randomly adjusted according to the physical differences, tumor site, and tumor size of the patient to obtain optimal electric field intensity and electric field coverage area for the tumor treatment, which can also allow the skin on the body surface of the patient where the insulated electrodes 600 are applied to breathe freely, thereby avoiding skin inflammation caused due to sweating and clogging of pores when heat is accumulated on the body surface of the patient and cannot be dissipated in a timely manner after long-duration tumor-treating fields therapy.

In addition, the flexible circuit board 611 of the insulated electrode 600 in this embodiment is only provided with one first conductive trace L1 electrically connected to the dielectric element 613, one second conductive trace L2 electrically connected to both the ground ends (not shown in the figure) of the two temperature sensors 614 and two third conductive traces L3 and L3′ electrically connected to the signal ends (not shown in the figure) of the two temperature sensors 614 respectively, to achieve the purpose of transmitting the alternating electrical signal of the electric field generator (not shown in the figure) to the dielectric element 613 through the first conductive trace L1, and achieve the purpose of applying the alternating electrical signal to the tumor site of the patient for tumor treatment. In addition, the flexible circuit board 611 is electrically connected to two temperature sensors 614 respectively through the second conductive trace L2 and the third conductive traces L3 and L3′ to achieve the signal transmission between the electric field generator (not shown in the figure) and the two temperature sensors 614, and therefore, a wiring design is less difficult, a structure is simple, a manufacture process is simplified, the flexible circuit board 611 is easy to manufacture, and the product manufacture yield rate is high, which can greatly reduce the manufacture costs.

### An Alternate Embodiment of the Sixth Embodiment of the Insulated Electrode

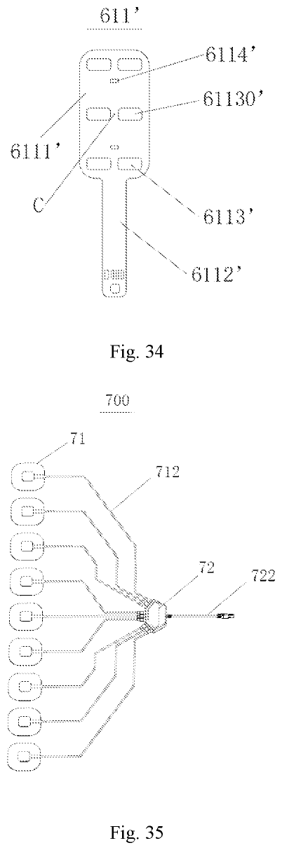

FIG. 32 to FIG. 34 show an insulated electrode 600′ in a variant embodiment of the sixth embodiment. The insulated electrode 600′ basically has the same structure as the insulated electrode 600, and main differences therebetween are that the electrode element 610′ of the electrical functional component 61′ of the insulated electrode 600′ is rectangular, and the electrode element 610′ includes a main body 6111′ of the flexible circuit board 611′, an insulating plate 612′, a dielectric element 613′, and two temperature sensors 614′ arranged on the main body 6111′ and located on the same side as the dielectric element 613′. The main body 6111′, the insulating plate 612′ and the dielectric element 613′ all have a rectangular sheet-shaped structure with four rounded corners. Preferably, the main body 6111′ is a rectangular sheet-shaped structure with a size of about 43.5 mm×23.5 mm. Arrangements of the conductive pad 6113′ and two pairs of pads 6114′ on the surface of the main body 6111′ are also slightly different. For details, refer to detailed description below. The flexible circuit board 611′ also includes a wiring portion 6112′ that is extended from the main body 6111′ and that is connected to the wire (not numbered), and a wiring manner of the flexible circuit board 611′ is the same as that of the flexible circuit board 611 in the sixth embodiment. Details are not described herein again.

The conductive pad 6113′ of the main body 6111′ includes six spaced conductive cores 61130′ with four conductive cores 61130′ respectively located at four corners of the main body 6111′ and two conductive cores 61130′ located in the middle of two long sides of the main body 6111′. Each conductive core 61130 has a rectangular structure with a size of approximately 8 mm×4 mm. Preferably, each conductive core 61130′ has a rectangular structure with four rounded corners. Six conductive cores 61130′ forming the conductive pad 6113′ are arranged into an array at intervals, and the six conductive cores 61130′ are arranged in three rows and two columns along a longitudinal direction of the main body 6111′. There are two conductive cores 61130′ in the first row, two conductive cores 61130′ in the middle row, and two conductive cores 61130′ in the last row. A gap between the two columns of conductive cores 61130 is about 2.4 mm, and gaps between adjacent rows of conductive cores 61130 are all about 12.8 mm. The six conductive cores 61130′ constituting the conductive pad 6113′ are both center-symmetrically and axial-symmetrically arranged, and each conductive core 61130′ is also axial-symmetrically. Therefore, when the six conductive cores 61130′ of the main body 6111′ are welded to the dielectric element 613′, stress balance between welding points is ensured, overall welding balance of the dielectric element 613′ is ensured, and welding quality is improved, which prevents a welding joint with a larger distance between the dielectric element 613′ and the main body part 6111′ from being weak and prone to break because the dielectric element 613′ is tilted when the welding stress is unbalanced, and can also avoid affecting fitting of the electrode patch 100.

The six conductive cores 61130′ of the conductive pad 6113′ are arranged at intervals, and a gap C is formed between each two adjacent conductive cores 61130′. Each two of the four conductive cores 61130′ in adjacent rows are arranged at intervals, and four gaps C between the four conductive cores 61130′ are connected with each other in a shaped of “+”. A dimension of the gap C each formed between two adjacent conductive cores 61130′ in the same column is greater than a dimension of the gap C each formed between two conductive cores 61130′ in the same row. Seven gaps C are formed between six conductive cores 61130′. The even gaps C are substantially connected in a shape of “#”. Adjacent gaps C are also connected with each other. In the seven gaps C, a straight line on which three gaps C each formed between the two adjacent conductive cores 61130′ in the same row has the same extension direction as the wiring portion 6112′.

The main body 6111′ is further provided with two pairs of pads 6114′ that are welded to the two temperature sensors 614′ respectively. Each pair of pads 6114′ are located at a corresponding connected region of four gaps C formed by four conductive cores 61130′ spaced apart in adjacent rows. A straight line on which a connecting line of respective centers of symmetry of the two pairs of pads 6114′ has the same extension direction as the wiring portion 6112′. A straight line on which a connecting line of two pairs of centers of symmetry of the two pairs of pads 6114′ is overlapped with a longitudinal long axis of the main body 6111′. A straight line on which a connecting line of two pairs of centers of symmetry of the two pairs of pads 6114′ is overlapped with a longitudinal long axis of the conductive pad 6113′. The four conductive cores 61130′ in the first row and the middle row are center-symmetrically arranged, and the four conductive cores 61130′ in the middle row and the last row are also center-symmetrically arranged. Each pair of pads 6114′ are arranged away from centers of symmetry of the four conductive cores 61130′ in the two adjacent rows. Specifically, a pair of pads 6114′ are located on a side, away from the wiring portion 6112′, of a center of symmetry of a rectangle formed by the four conductive cores 61130′ in the first and middle rows. Another pair of pads 6114′ are located on a side, closer to the wiring portion 6112′, of a center of symmetry of a rectangle formed by the four conductive cores 61130′ in the middle and last rows.

Based on the insulated electrode 600, 600′ in the present disclosure, because single electrode element 610, 610′ is used to apply an alternating electrical signal to the tumor site of the patient, when the insulated electrode 600 and 600′ fail to operate normally, only the insulated electrodes 600 and 600′ with single electrode element 610, 610′ need to be replaced without a need to scrap the insulated electrodes with multiple electrode elements 610, 610′ in the entire region, so that costs of the tumor treatment for the patient can be reduced. In addition, random quantities of the insulated electrodes 600, 600′ in the present disclosure can be combined based on the tumor site of the patient and the size of the tumor site of the patient, so as to ensure the coverage areas of the insulated electrodes 600, 600′ for the tumor-treating fields therapy and ensure the electric field treatment intensity. In addition, the relative positions of the multiple insulated electrodes 600 and 600′ can also be randomly adjusted according to the physical differences, tumor site, and tumor size of the patient to obtain optimal electric field intensity and electric field coverage area for the tumor treatment, which can also allow the skin on the body surface of the patient where the insulated electrodes 600, 600′ are applied to breathe freely, thereby avoiding skin inflammation caused due to sweating and clogging of pores when heat is accumulated on the body surface of the patient and cannot be dissipated in a timely manner after long-duration tumor-treating fields therapy. In addition, the flexible circuit board 61 of the insulated electrode 600 in this embodiment is only provided with a first conductive trace L1 electrically connected to the dielectric element 613, a second conductive trace L2 electrically connected to both the ground ends (not shown in the figure) of the two temperature sensors 614 and two third conductive traces L3, L3′ electrically connected to the signal ends (not shown in the figure) of the two temperature sensors 614 respectively, to achieve the purpose of transmitting the alternating electrical signal of the electric field generator (not shown in the figure) to the dielectric element 613 through the first conductive trace L1, and achieve the purpose of applying the alternating electrical signal to the tumor site of the patient for tumor treatment. In addition, the flexible circuit board 61 is electrically connected to two temperature sensors 614 respectively through the second conductive trace L2 and the third conductive trace L3, L3′ to achieve the signal transmission between the electric field generator (not shown in the figure) and the two temperature sensors 614, and therefore, a wiring design is less difficult, a structure is simple, a manufacture process is simplified, the flexible circuit board is easy to manufacture, and the product manufacture yield is high, which can greatly reduce the manufacture costs.

### A Seventh Embodiment of the Insulated Electrode

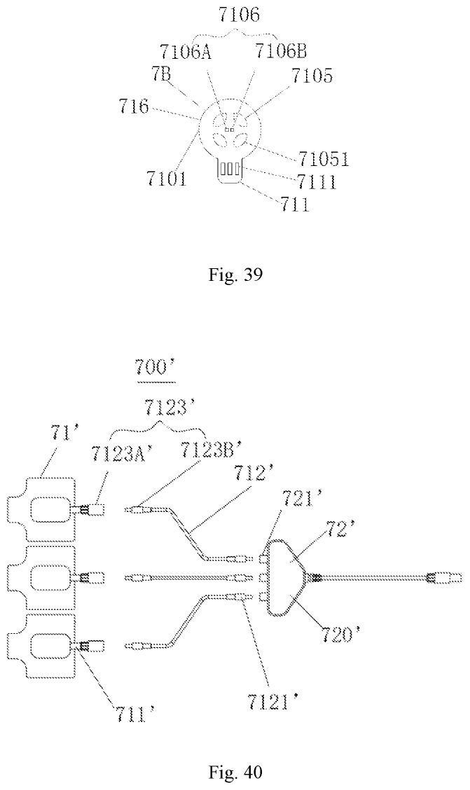

FIG. 35 to FIG. 39 show an embodiment of an insulated electrode 700. The insulated electrode 700 includes an electrical connector 72 electrically connected to an electric field generator (not shown in the figure) or an adapter (not shown in the figure) and multiple electrode segments 71 detachably assembled as a group on the electrical connector 72. The electrode segment 71 in this embodiment may also be the insulated electrodes 600 and 600′ in the sixth embodiment. The multiple electrode segments 71 of the insulated electrode 700 are detachably assembled into the electrical connector 72, and the multiple electrode segments 71 are connected to the electrical connector 72 in parallel, so that a damaged electrode segment 71 is easy to replace when the specific electrode segment 71 is damaged and cannot operate, without needing to scrap all the multiple electrode segments 71, which can reduce the manufacturing costs, avoid waste, and ensure sufficient electric field intensity during the tumor-treating fields therapy. In addition, a random quantity of electrode segments 71 can be combined and positions of the electrode segments 71 can be randomly adjusted based on the body difference, tumor site and tumor size of the patient, so as to ensure that the intensity of the electric field applied to the tumor site of the patient is most appropriate. In addition, application positions and gaps between the multiple electrode segments 71 can also be randomly adjusted according to a condition of the patient, which can allow the skin on the tumor site of the patient to breathe freely, thereby avoiding skin inflammation caused due to sweating and clogging of pores when generated heat is rapidly accumulated on a position of the tumor site of the patient to which the electrode segment 71 is applied and cannot be dissipated in a timely manner after long-duration electric field treatment.

Referring to FIG. 37 to FIG. 39 specially, the multiple electrode segments 71 can be detachably plugged into the corresponding parts of the electrical connector 72, so as to achieve the parallel connection between each electrode segment 71 and the electrical connector 72, and achieve the electrical connection to the electric field generator (not shown in the figure) through the electrical connector 72, which allows for timely and easy replacement of a specific electrode 71 when damaged, thereby ensuring that the electric field applied to the tumor site has sufficient strength and improving the therapeutic effect. Each electrode segment 71 includes a single electrode element 710 applying an alternating electric signal to the tumor site of the patient, a wiring portion 711 connected to the electrode element 710, a first wire 712 welded to the wiring portion 711, an adhesive-backed layer 713 adhered to the electrode element 710, a supporting member 714 surrounding the electrode element 710 and adhered to the adhesive-backed layer 713, and an adhesive member 715 covering the corresponding parts of the electrode element 710 and the supporting member 714. The first wire 712 has one end welded to the wiring portion 711 and the other end detachably plugged into the electrical connector 72 through the first plug 7121 located at the end thereof, so as to achieve the electrical connection between the electrode element 710 and the electrical connector 72, thereby further transmitting an alternating electric signal generated by the electric field generator (not shown in the figure) to the electrode element 710 through the electrical connector 72 for tumor-treating fields therapy. Optionally, the electrode segment 71 can be directly plugged into the electric field generator (not shown in the figure) through the first plug 7121 of the first wire 712 or plugged into an adapter (not shown in the figure) first, and then is electrically connected to the electric field generator (not shown in the figure) through the adapter (not shown in the figure) to achieve the electrical connection between the electrode segment 71 and the electric field generator (not shown in the figure).

The electrode element 710 of the electrode segment 71 includes a main body 7101 arranged at the end of the wiring portion 711 and electrically connected to the wiring portion 711, an insulating plate 7102 and a dielectric element 7103 arranged on opposite sides of the main body 7101 respectively, and a temperature sensor 7104 arranged on the main body 7101 and located on the same side as the dielectric element 7103. The entire electrode element 710 has a circular sheet-shaped structure. The main body 7101, the insulating plate 7102, and the dielectric element 7103 are all circular sheet-shaped structures, and have approximately the same size, and are arranged in one-to-one correspondence along a thickness direction. Centers of the main body 7101, the insulating plate 7102 and the dielectric element 7103 are located on the same straight line.

A conductive pad 7105 is provided on a side of the main body 7101 facing toward the human skin. A conductive pad 7105 is welded to the corresponding dielectric element 7103 to mount the dielectric elements 7103 onto the main body 7101. The conductive pad 7105 can be completely covered by the dielectric element 7103, so that the conductive pad 7105 and the dielectric element 7103 can be welded through soldering tin (not shown in the figure). The center of the conductive pad 7105 is located on a center line of the main body 7101. The conductive pad 7105 includes multiple conductive cores 71051 center-symmetrically arranged, which can effectively prevent the position of the dielectric element 7103 from being shifted due to accumulation of soldering tin (not shown includes in the figure) during the welding process. Top surfaces of multiple conductive cores 71051 are located in the same plane, so that a rosin joint during welding of the dielectric element 7103 can be avoided. A pair of pads 7106 are further provided between the multiple conductive cores 71051, and can be electrically connected to corresponding parts of the corresponding temperature sensor 7104 to implement the electrical connection between the temperature sensor 7104 and the main body 7101. Two pads 7106 include a first pad 7106A and a second pad 7106B. The temperature sensor 7104 has a signal end (not shown in the figure) and a ground end (not shown in the figure). The first pad 7106A is welded to the ground end (not shown in the figure) of the temperature sensor 7104, and the second pad 7106B is welded to the signal end (not shown in the figure) of the temperature sensor 7104.

The insulating plate 7102 is made of an insulated material. Preferably, the insulating plate 7102 is an epoxy glass cloth laminate. The insulating plate 7102 is adhered to a surface of the main body 7101 that is away from human skin through a sealant (not shown in the figure), which not only can enhance the strength of the main body 7101 and provide a flat welding plane for a welding operation between the main body 7101 and the dielectric element 7103, but also can further prevent water vapor in the air on the side of the electrode segment 71 that is away from the skin coming into contact with the soldering tin (not shown in the figure) between the main body 7101 and the dielectric element 7103, and prevent an electrical connection between the main body 7101 and the dielectric element 7103 from being affected because the water vapor corrodes the soldering tin (not shown in the figure) between the main body 7101 and the dielectric element 7103. The size of the insulating plate 7102 is approximately the same as that of the main body 7101, so as to avoid a void generated in the sealant (not shown in the figure) because filling of the sealant (not shown in the figure) in the gap (not shown in the figure) formed by welding the dielectric element 7103 to the main body 7101 is affected if the sealant (not shown in the figure) overflows to the side of the main body 7101 facing toward the human skin due to a capillary effect when the insulating plate 7102 is adhered to the side of the main body 7101 that is away from human skin through the sealant (not shown in the figure), thereby avoiding damage of the product caused because a popcorn effect occurs if water vapor expands rapidly and causes an explosion due to a large difference between thermal expansion coefficients of the water vapor and the sealant (not shown in the figure) in the void during curing of the sealant (not shown in the figure) at high temperature.

The dielectric element 7103 is made of a material with a high dielectric constant, which has a property of impeding conduction of the direct current and allowing conduction of the alternating current, thereby ensuring safety of a user during the tumor-treating fields therapy. Preferably, the dielectric element 7103 is a dielectric ceramic plate. The dielectric element 7103 is provided with a through-hole 71031 corresponding to a pair of pads 7106 of the main body 7101 in the middle thereof to accommodate the temperature sensor 7104. A metal layer (not shown in the figure) is attached to the surface of the dielectric element 7103 facing toward the main body 7101. A point-to-side welding is formed between the metal layer (not shown in the figure) of the dielectric element 7103 and the conductive cores 71051 of the conductive pad 7105 of the main body 7101, without requiring high welding alignment accuracy, thereby facilitating welding. The inner edge of the metal layer (not shown in the figure) of the dielectric element 7103 and the through-hole 71031 of the dielectric element 7103 are arranged at intervals, which can avoid a short circuit of the temperature sensor 7104 that is caused because the soldering tin (not shown in the figure) between the metal layer (not shown in the figure) of the dielectric element 7103 and the main body 7101 is melted and spreads into the through-hole 71031 of the dielectric element 7103 when heated. The outer edge of the metal layer (not shown in the figure) of the dielectric element 7103 and the outer edge of the dielectric element 7103 are also arranged at intervals, which can prevent the soldering tin (not shown in the figure) between the metal layer (not shown in the figure) of the dielectric element 7103 and the main body 7101 from melting and overflowing to the outerside of the main body 7101 when heated, and prevent the direct current unimpeded by the dielectric element 7103 from passing through and directly acting on the body surface of the patient when the electrode segment 71 is applied to the body surface corresponding to the tumor site of the patient.

The gap (not shown in the figure) formed by welding the dielectric element 7103 and the main body 7101 is filled with the sealant (not shown in the figure) to protect the soldering tin (not shown in the figure) between the dielectric element 7103 and the main body 7101, thereby preventing the alternating electric signal being unable to be applied to the tumor site of the patient through the dielectric element 7103 because the welding joint is broken when the dielectric element 7103 is affected by external force, and further preventing an electrical connection between the dielectric element 7103 and the main body 7101 being affected because the water vapor in the air enters the gap (not shown in the figure) and corrodes the soldering tin (not shown in the figure) between the dielectric element 7103 and the main body 7101. The outer diameter of the dielectric element 7103 is slightly less than the diameter of the main body 7101, and the sealant (not shown in the figure) is filled inside the gap (not shown in the figure) along the edge of the main body 7101 on the outer side of the dielectric element 7103 through a capillary phenomenon when the sealant (not shown in the figure) is filled, so as to facilitate the filling of the sealant (not shown in the figure) into the gap (not shown in the figure) formed by the welding the dielectric element 7103 to the main body 7101. When the gap (not shown in the figure) formed by welding the dielectric element 7103 to the main body 7101 is filled with the sealant (not shown in the figure), the air in the gap (not shown in the figure) can be discharged through the through-hole 71031 of the dielectric element 7103, so as to avoid the void generated in the sealant (not shown in the figure) filled in the gap (not shown in the figure), thereby improving quality of the product.

The temperature sensor 7104 is arranged on the main body 7101 through welding the ground end (not shown in the figure) thereof to the first pad 7106A arranged on the main body 7101 and welding the signal end (not shown in the figure) thereof to the second pad 7106B arranged on the main body 7101. After being welded to the main body 7101, the temperature sensor 7104 is accommodated in the through-hole 71031 of the dielectric element 7103. Preferably, the temperature sensor 7104 is a thermistor. The temperature sensor 7104 is configured to monitor the temperature of the adhesive member 715 covering one surface of the dielectric element 7103 of the electrode element 710 facing toward the human skin, to further detect temperature of the human skin to which the adhesive member 715 is attached. When the temperature monitored by the temperature sensor 7104 exceeds an upper safe temperature limit of the human body, the tumor electric field therapy system can promptly reduce or cut off the alternating electrical signal applied to the electrode segment 71 by the electric field generator (not shown in the figure) to avoid a low-temperature burn to the human body. The temperature sensor 7104 is welded to the main body 7101 through a pair of pads 7106 of the main body 7101 and then sealed with the sealant (not shown in the figure) to prevent water vapor from corroding the temperature sensor 7104 and causing the temperature sensor 7104 to fail.

The wiring portion 711 is arranged by laterally extending the main body 7101 of the electrode element 710. A welding joint between the wiring portion 711 and the first wire 712 is externally covered with a heat shrinkable sleeve 7122. The heat shrinkable sleeve 7122 insulates and protects the joint between the first wire 712 and the wiring portion 711 and provides support, thereby avoiding breakage of the joint between the first wire 712 and the wiring portion 711 and further achieving dust-proof and waterproof purposes.

The main body 7101 of the electrode element 710 and the wiring portion 711 jointly form the flexible circuit board 716 of the electrode segment 71. From the perspective of formation of the electrode element 710, the insulating plate 7102 is arranged on the side of the main body 7101 of the flexible circuit board 716 that is away from the human skin, the dielectric element 7103 is arranged on the side of the main body 7101 of the flexible circuit board 716 facing toward the human skin, and the temperature sensor 7104 is arranged on the side of the main body 7101 of the flexible circuit board 716 facing toward the human skin.

The flexible circuit board 716 includes an insulating substrate 7B and multiple conductive traces (not shown in the figure) embedded in the insulating substrate 7B. That is, the main body 7101 of the electrode element 710 and the wiring portion 711 both have an insulating substrate 7B and multiple conductive traces (not shown in the figure) embedded in the insulating substrate 7B. Multiple conductive traces (not shown in the figure) in the insulating substrate 7B of the main body 7101 are respectively and electrically connected to multiple corresponding conductive traces (not shown in the figure) in the insulating substrate 7B of the wiring portion 711. In this embodiment, the flexible circuit board 716 has three conductive traces (not shown in the figure), including a conductive trace (not shown in the figure) that connects all the conductive cores 71051 of the conductive pad 7105 of the main body 7101 in series, a conductive trace (not shown in the figure) electrically connected to the ground end (not shown in the figure) of the temperature sensor 7104 on the main body 7101, and a conductive trace (not shown in the figure) electrically connected to the signal end (not shown in the figure) of the temperature sensor 7104 on the main body 7101, respectively. Three golden fingers 7111 are provided on one side of the wiring portion 711 facing toward the human skin. The three golden fingers 7111 of the wiring portion 711 are electrically connected to three conductive traces (not shown in the figure), respectively. The three golden fingers 7111 of the wiring portion 711 are welded to one end of the first wire 712 away from the first plug 7121, to achieve the electrical connection between the main body 7101 of the electrode element 710 and the first wire 712, and further achieve the electrical connection between the dielectric element 7103, the temperature sensor 7104 and the first wire 712 through the main body 7101.

The conductive cores 71051 are exposed from the insulating substrate 7B of the main body 7101. The insulating substrate 7B of the flexible circuit board 716 can isolate water vapor in the air around the electrode segment 71 from the soldering tin (not shown in the figure) between the conductive pad 7105 and the dielectric element 7103, and prevent water vapor in the air on the side away from the skin corroding the soldering tin (not shown in the figure) between the conductive pad 7105 on the main body 7101 of the flexible circuit board 716 and the dielectric element 7103. The insulating substrate 7B of the flexible circuit board 716 and the insulating plate 7102 have a function of double isolation, which can prolong service life of the electrode segment 71.

The adhesive-backed layer 713 is arranged in a shape of a sheet and is mainly made of a material with flexibility, breathability, and insulation. The adhesive-backed layer 713 is a mesh fabric. Specifically, the adhesive-backed layer 713 is a mesh-shaped nonwoven fabric, has characteristics of softness, lightness, moisture-proofing and breathability, and can still keep the skin surface of the patient dry when applied to the body surface of the patient for a long time. A surface of the adhesive-backed layer 713 facing toward the body surface of the patient is also coated with a biocompatible adhesive (not shown in the figure) for tightly fitting the adhesive-backed layer 713 to the corresponding body surface corresponding to the tumor site of the patient. In this embodiment, only one electrode element 710 is adhered to the adhesive-backed layer 713. The adhesive-backed layer 713 is approximately a cube sheet-shaped structure. Four corners of the adhesive-backed layer 713 have rounded angles.

The supporting member 714 is adhered to the adhesive-backed layer 713 and encloses the periphery of the electrode element 710. The supporting member 714 has a through-hole 7141 in the middle thereof, to accommodate the electrode element 710. The supporting member 714 may be made of a foam material. The supporting member 714 is flush with a side surface of the electrode element 710 away from the adhesive-backed layer 713. That is, the supporting member 714 is flush with a side surface of the electrode element 710 facing toward the adhesive member 715.

The adhesive member 715 has adhesiveness on both sides. A surface of the adhesive member 715 is adhered to side surfaces of the supporting member 714 and the electrode element 710 that are away from the adhesive-backed layer 713. The other surface of the adhesive member 715 is used as an adhesion layer to be adhered to epidermis of the human, to keep the skin surface moist and relieve local pressure. Preferably, the adhesive member 14 may use conductive hydrogel to serve as a conductive medium. Under the support effect of the supporting member 714, the adhesive member 715 has better adhesion to human skin.

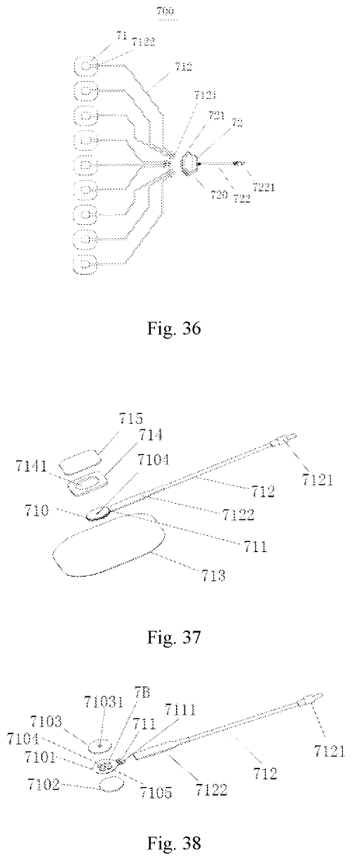

Referring to FIG. 36 and FIG. 37 specifically, the electrical connector 72 is provided with multiple sockets 721 into which the first plug 7121 of the first wire 712 of the corresponding electrode segment 71 is plugged and a second wire 722 plugged into an adapter (not shown in the figure) or an electric field generator (not shown in the figure). The second wire 722 is provided with a second plug 7221 at one end away from the electrical connector 72 and can be directly plugged into the electric field generator (not shown in the figure) or plugged into an adapter (not shown in the figure) first, and then is plugged to the electric field generator (not shown in the figure) through the adapter (not shown in the figure) to achieve the electrical connection to the electric field generator (not shown in the figure). The multiple sockets 721 and the second wire 722 are respectively arranged at opposite ends of the electrical connector 72. The electrical connector 72 is plugged with the first plugs 7121 of the first wires 712 of the electrode segments 71 through the sockets 721 thereof, so as to connect the multiple electrode segments 71 to the electrical connector 72, respectively, and achieve the electrical connection between the multiple electrode segments 71 and the electrical connector 72. Then, by using a second plug 7221 plugging into an electric field generator (not shown in the figure) or an adapter (not shown in the figure), an electrical connection between the multiple electrode segments 71 and the electric field generator (not shown in the figure) is achieved. When used, the multiple electrode segments 71 are applied to the corresponding body surface corresponding to the tumor site of the patient, the multiple electrode segments 71 are plugged into the corresponding sockets 721 of the electrical connector 72 through the first plugs 7121, and the electrical connector 72 is electrically connected to the electric field generator (not shown in the figure) through the second plug 7221. In this way, the alternating electric signal generated by the electric field generator (not shown in the figure) is transmitted to the multiple electrode segments 71 through the electrical connector 72, and acts on the tumor site of the patient through the multiple electrode segments 71 to interfere with or prevent mitosis of tumor cells of the patient, so as to achieve the purpose of treating a tumor. The electrode segments 71 of the insulated electrode 700 in the present disclosure and the electrical connector 72 are detachably plugged, each electrode segment 71 includes only one electrode element 710, and each electrode element 710 is electrically connected to the electric field generator (not shown in the figure) through a first wire 712 that is electrically connected to the electrode element 710. When the electrode element 710 or the first wire 712 is damaged and cannot operate, only the corresponding electrode segment 71 needs to be replaced, which can reduce the costs of the tumor treatment for the patient. A random quantity of electrode segments 71 of the insulated electrode 700 can be further combined based on the tumor site and the tumor size of the patient, so as to ensure the coverage area of the insulated electrode 700 for the tumor-treating fields therapy and ensure the electric field intensity of the insulated electrode 700 for the tumor-treating fields therapy, thereby improving the effect of the tumor-treating fields therapy. In addition, the relative positions of the multiple electrode segments 71 of the insulated electrode 700 can also be randomly adjusted according to the physical differences, tumor site, and tumor size of the patient to obtain optimal electric field intensity and electric field coverage area for the tumor treatment, which can also allow the skin on the body surface of the patient where the electrode segments 71 are applied to breathe freely, thereby avoiding skin inflammation caused due to sweating and clogging of pores when heat is accumulated on the body surface of the patient and cannot be dissipated in a timely manner after long-duration tumor-treating fields therapy.

In this embodiment, the number of sockets 721 of the electrical connector 72 is nine, and the number of the electrode segments 71 is nine. The electrical connector 72 is provided with a body 720, and the body 720 has substantially a polyhedral structure. In this embodiment, the body 720 has substantially a hexagonal prism structure. The nine sockets 721 are respectively located on multiple adjacent side surfaces of the body 720, and an obtuse angle is formed between the adjacent side surfaces of the body. The second wire 722 is arranged on the side of the body 720 away from the socket 721. In this embodiment, nine sockets 721 are evenly arranged on three adjacent side surfaces of the body 720, and every three sockets 721 are arranged on the same side surface of the body 720 of the electrical connector 72. Terminals (not shown in the figure) in the nine sockets 721 of the electrical connector 72 may be connected in series, so that the nine electrode segments 71 are connected in series to each other. Terminals (not shown in the figure) in the nine sockets 721 of the electrical connector 72 may also be connected in parallel, so that the nine electrode segments 71 are connected in parallel to each other. When the terminals (not shown in the figure) in the sockets 721 of the electrical connector 72 are connected in series, all the electrode segments 71 need to be plugged into the electrical connector 72 for use. When the terminals (not shown in the figure) in the sockets 721 of the electrical connector 72 are connected in parallel, some electrode segments 71 can be selectively plugged into the electrical connector 72 based on needs for more convenient and flexible use. Optionally, the terminals (not shown in the figure) in the nine sockets 721 of the electrical connector 72 may be connected partially in series and partially in parallel. The terminals (not shown in the figure) in the sockets 721 of the electrical connector 72 may be connected in series, or in parallel, or partially in series and partially in parallel based on needs, so that the multiple electrode segments 71 connected to the electrical connector 72 are connected all in series, or all in parallel, or partially in series and partially in parallel. When the tumor size is large, an appropriate number of electrode segments 71 can be selected and the gap between the electrode segments 71 can be freely adjusted based on the needs to ensure a coverage area of the insulated electrode 700 for the tumor-treating fields therapy and an electric field treatment effect. When the tumor position is closer to the side of the corresponding part of the body, the number of applied electrode segments 71 of the insulated electrode 700 can be appropriately increased on the corresponding body surface away from the tumor, to enhance the intensity of the electric field on the side away from the tumor.

### A Variant Embodiment of the Insulated Electrode of the Seventh Embodiment

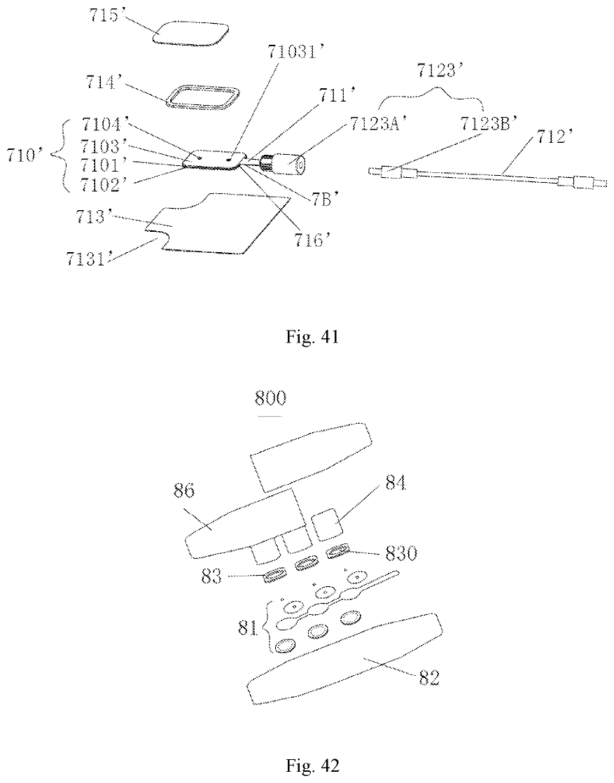

FIG. 40 and FIG. 41 show an insulated electrode 700′ which is a variation embodiment of of the insulated electrode 700 described in the seventh embodiment. The insulated electrode 700′ also includes multiple electrode segments 71′ that apply an alternating electric signal to the tumor site of the patient and an electrical connector 72′ that is electrically connected to an adapter (not shown in the figure) or an electric field generator (not shown in the figure). The multiple electrode segments 71′ are detachably plugged into the electrical connector 72′ as a group, to achieve the electrical connection between the multiple electrode segments 71′ and the electrical connector 72′, thereby realizing the electrical connection with the electric field generator (not shown in the figure) through the electrical connector 72′. Each electrode segment 71′ includes an electrode element 710′, a wiring portion 711′ connected to the electrode element 710′, a first wire 712′ welded to the wiring portion 711′, an adhesive-backed layer 713′ adhered to the electrode element 710′, a supporting member 714′ surrounding the electrode element 710′ and adhered to the adhesive-backed layer 713′, and an adhesive member 715′ covering the corresponding parts of the electrode element 710′ and the supporting member 714′. A difference between the insulated electrode 700′ in the present embodiment and the insulated electrode 700 in the seventh embodiment is that: the insulated electrode 700′ includes three electrode segments 71′, a body 720′ of the electrical connector 72′ is substantially a triangular prism structure, the electrical connector 72′ is provided with three sockets 721′, and the three sockets 721′ are all arranged on the same side surface of the body 720′ of the electrical connector 72′. The wiring portion 711′ of each electrode segment 71′ is connected to the corresponding first wire 712′ in a detachable plug-in method. The wiring portion 711′ of the electrode segment 71′ is electrically connected to the first wire 712′ via a plug-in member 7123′. The plug-in member 7123′ includes a docking socket 7123A′ and a docking plug 7123B′. The docking socket 7123A′ is connected to the wiring portion 711′, and the docking plug 7123B′ is connected to the end of the first wire 712′ away from the first plug 7121′. That is, the docking socket 7123A′ is arranged at the end of the wiring portion 711′, and the docking plug 7123B′ is arranged at the end of the first wire 7121′ away from the first plug 7121′. The docking socket 7123A′ and the electrode element 710′ are located at opposite ends of the wiring portion 711′. The docking plug 7123B′ and the first plug 7121′ are arranged at opposite ends of the first wire 712′. When the electrode element 710′ of the electrode segment 71′ is unable to operate due to damage, only the part of the electrode segment 71′ except the first wire 712′ need to be replaced, and the first wire 712′ can still be used, further reducing the costs of tumor treatment for patients.

The adhesive-backed layer 713′ of the electrode segment 71′ has an approximately convex structure. The adhesive-backed layer 713′ has two concave angles 7131′ formed by respectively recessing two corners inward. The two concave angles 7131′ are respectively at two corners of the adhesive-backed layer 713′ away from the wiring portion 711′. The concave corner 7131′ of the adhesive-backed layer 713′ communicates with the external environment and is arranged in a shape of “L”. An angle between the two sides of the adhesive-backed layer 713′ that form the concave angle 7131′ is greater than or equal to 90 degrees and therefore, when the electrode segment 71′ is applied to the body surface corresponding to the tumor site of the patient, arching and folds at the corner of the adhesive-backed layer 713′ can be avoided, so as to avoid a low-temperature scald caused because the electrode element 710′ generates more heat when impedance between the electrode element 710′ and the skin is increased if air enters space between the electrode element 710′ and the skin from the folds.

The electrode segment 71′ is similar to the insulated electrode 600′ in the variation embodiment of the sixth embodiment, and has an electrode element 710′ with an approximately rectangular sheet-shaped structure. The main body 7101′ of the electrode element 710′, the insulating plate 7102′, and the dielectric element 7103′ all have a rectangular sheet-shaped structure. Two temperature sensors 7104′ are arranged on one side of the main body 7101′ where the dielectric element 7103′ is arranged. The dielectric element 7103′ is provided with two through-holes 71031 for accommodating the temperature sensors 7104′. The two temperature sensors 7104′ are symmetrically arranged on the main body 7101′, which can detect temperature of corresponding human skin at different positions, thereby ensuring accuracy of detected data. Four conductive traces (not shown in the figure) are embedded in the insulating substrate 7B′ of the flexible circuit board 716′ formed by the main body 7101′ of the electrode element 710′ and the wiring portion 711′. Four conductive traces (not shown in the figure) of the flexible circuit board 716′ are respectively a conductive trace (not shown in the figure) that connects all the conductive cores (not shown in the figure) of the conductive pad (not shown in the figure) of the main body 7101′ in series, a conductive trace (not shown in the figure) that connects ground ends (not shown in the figure) of two temperature sensors 7104′ on the corresponding main body 7101′ in series, and two conductive traces (not shown in the figure) that connects signal ends (not shown in the figure) of two temperature sensors 7104′ in parallel. Four golden fingers (not shown in the figure) are provided on one side of the wiring portion 711′ facing toward the human skin. Four conductive traces (not shown in the figure) are electrically connected to four golden fingers (not shown in the figure) of the wiring portion 711′.

At least one of electrode segments 71, 71′ of the insulated electrodes 700, 700′ in the tumor electric field therapy system 1000 in the application is detachably plugged into the electric field generator (not shown in the figure) through the first wires 712, 712′ arranged on the electrode segments 71, 71′, or first detachably plugged into the adapter (not shown in the figure) and then electrically connected to the electric field generator (not shown in the figure) through the adapter (not shown in the figure), or detachably plugged into the electrical connector 72, 72′ and then electrically connected to the electric field generator (not shown in the figure) through the electrical connector 72, 72′, to achieve the electrical connection between the electrode segment 71, 71′ and the electric field generator (not shown in the figure). In addition, each of the electrode segments 71, 71′ only contains one of electrode element 710, 710′ electrically connected to the corresponding first wire 712, 712′, and when the electrode element 710, 710′ is damaged and cannot operate, only the corresponding electrode segment 71, 71′ need to be replaced, which can reduce costs of the tumor treatment for the patient. In addition, random quantities of electrode segments 71,71′ of the insulated electrode 700, 700′ in the present disclosure can be combined and positions of the electrode segments 71,71′ can be randomly adjusted based on the tumor site, tumor position and tumor size of the patient, so as to ensure the coverage areas of the insulated electrode 700, 700′ for the tumor-treating fields therapy and ensure the electric field intensity of the insulated electrode 700, 700′ for the tumor-treating fields therapy, thereby improving the effect of the electric field treatment. In addition, the relative gaps between the electrode segments 71, 71′ of the insulated electrode 700, 700′ can allow the skin on the body surface of the patient to breathe freely and exchange heat with external air, thereby avoiding skin diseases caused due to sweating and clogging of pores when heat is accumulated on the body surface of the patient after long-duration electric field treatment.

### A Eighth Embodiment of the Insulated Electrode

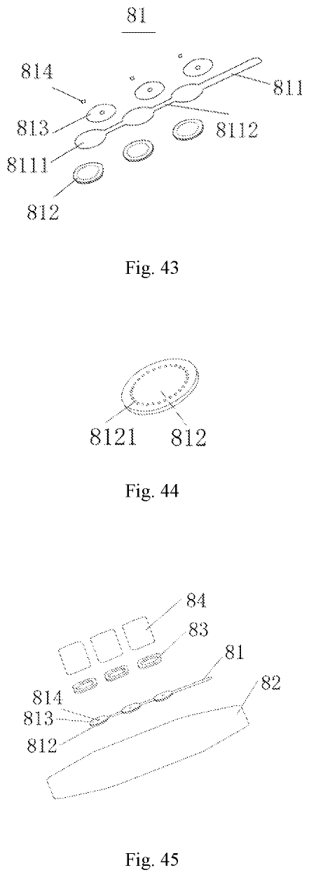

Referring to FIG. 42 to FIG. 45, the insulated electrode 800 includes an electrical functional component 81, a flexible adhesive-backed layer 82, several supporting members 83, several adhesive members 84, and release papers 86 disposed on the adhesive members 84 and fits the adhesive-backed layer 82. The electrical functional component 81 includes a flexible circuit board 811, a plurality of heat dissipation reinforcing members 812 and a plurality of dielectric elements 813 respectively provided on two opposite sides of the flexible circuit board 811, and a plurality of temperature sensors 814. The flexible circuit board 811 has several main bodies 8111 and several connection portions 8112 connected to the main bodies 8111. A specific structure of the electrical functional component 81 is basically the same as that of the electrical functional component in the previous embodiment. Only one difference is that: the insulating plate in the previous embodiment is replaced with the heat dissipation reinforcing member 812 to improve heat dissipation performance. The following description only focuses on the heat dissipation reinforcing member 812, and other structures in the electrical functional component 81 are not described again.

Each heat dissipation reinforcing member 812 is arranged substantially in a shape of a circular sheet, one side of the heat dissipation reinforcing member 812 closer to the body surface of the patient is adhered to the main body 8111 of the flexible circuit board 811 via an adhesive (not shown in the figure), and the other side of the heat dissipation reinforcing member 812 away from the body surface of the patient is adhered to the adhesive-backed layer 82 through a biocompatible adhesive arranged on the adhesive-backed layer 82. Each heat dissipation reinforcing member 812 is arranged on one side of the main body 8111 of the flexible circuit board 811 away from the body surface of the patient, and is used to support the corresponding main body 8111 of the flexible circuit board 811, to weld the temperature sensor 814 and the dielectric element 813 to the main body 8111 of the flexible circuit board 811. Each heat dissipation reinforcing member 812 is sandwiched between the adhesive-backed layer 82 and the main body 8111 of the flexible circuit board 811 after the electrical functional component 81 are arranged on the adhesive-backed layer 82. The diameter of the heat dissipation reinforcing member 812 is approximately the same as the diameter of the main body 8111 of the flexible circuit board 811. The number of heat dissipation reinforcing members 812 is consistent with the number of main bodies 8111 of the flexible circuit board 811. The number of heat dissipation reinforcing members 812 is consistent with the number of dielectric elements 813. The heat dissipation reinforcing member 812 is insulated from the conductive pad (not shown in the figure) on the main body 8111 of the flexible circuit board 811. The heat dissipation reinforcing member 812 is electrically insulated from the flexible circuit board 811.

The heat dissipation reinforcing member 812 is made of a material with a thermal conductivity greater than 200 W/mK, which can quickly dissipate heat generated by a conductive pad (not shown in the figure) and the dielectric element 813 exposed on a side, closer to a body surface of a patient, of the main body 8111 of the flexible circuit board 811 for applying an alternating electric field, and dissipate heat accumulated on the body surface of the patient because the insulated electrode 800 applies an alternating electric signal to the body surface corresponding to the tumor site of the patient for a long time, thereby enhancing the heat dissipation performance of the insulated electrode 800 during tumor-treating fields therapy, and ensuring a good therapeutic effect of the tumor alternating electric field treatment by prolonging treatment time while keeping magnitude of the alternating electric field applied to the insulated electrode 800 unchanged.

The heat dissipation reinforcing member 812 may be a metal plate, a metal alloy plate, or a graphene composite plate. The heat dissipation reinforcing member 812 is further provided with one or more heat dissipation holes 8121, which can further quickly dissipate heat through the adhesive-backed layer 82. The heat dissipation holes 8121 are evenly distributed on the heat dissipation reinforcing member 812. The heat dissipation hole 8121 is set to be circular. Preferably, the heat dissipation reinforcing member 812 is a metal plate or metal alloy plate with a thickness of 0.1 mm to 0.7 mm. Specifically, the heat dissipation reinforcing member 812 is an aluminium plate or aluminium alloy plate with a thickness of 0.3 mm to 0.6 mm. Specifically, the heat dissipation reinforcing member 812 is an aluminium plate with a thickness of 0.6 mm. The number of heat dissipation holes 8121 evenly distributed on the aluminum plate is 30, and the diameter of the heat dissipation hole 8121 is 0.5 mm. Alternatively, the heat dissipation reinforcing member 812 is 6063 aluminum alloy plate with a thickness of 0.3 mm. A thermal conductivity of 6063 aluminum alloy is 201 W/mK. The number of heat dissipation holes 8121 evenly distributed on the 6063 aluminum alloy plate is 50, and the diameter of the heat dissipation hole 8121 is 0.4 mm. Alternatively, the heat dissipation reinforcing member 812 is made of a graphene composite material with a thickness of 0.1 mm. The heat dissipation reinforcing member 812 has a thermal conductivity greater than 300 W/mK.

The adhesive-backed layer 82 is arranged in a shape of a sheet and is mainly made of a material with flexibility, breathability, and insulation. The adhesive-backed layer 82 has several ventilation holes (not shown in the figure) penetrating therethrough, so that hair follicles and sweat glands in the skin on the body surface of the patient that is covered by the adhesive-backed layer 82 can breathe freely when the adhesive-backed layer 82 is applied to the body surface of the patient, to avoid skin inflammation caused because a superficial layer of the skin of the patient is damaged when the sweat glands and hair follicles on the body surface of the patient covered by the adhesive-backed layer 82 are clogged. The adhesive-backed layer 82 is a mesh fabric. Specifically, the adhesive-backed layer 82 is a mesh-shaped nonwoven fabric. A surface of the adhesive-backed layer 82 facing toward the body surface of the patient is also coated with a biocompatible adhesive for tightly fitting the adhesive-backed layer 82 to a target region on the body surface of the patient.

Each supporting member 83 is approximately set to be opened in the middle and ring-shaped and has a through-hole 830 to allow the dielectric element 813 to pass through. The thickness of the supporting member 83 is approximately the same as that of the dielectric element 813. A plane where a top of the supporting member 83 is located is at the same vertical height as the surface on the side of the dielectric element 813 facing toward the body surface of the patient, that is, the surface on the side of the supporting member 83 closer to the body surface of the patient is coplanar with the surface on the side of the dielectric element 813 closer to the body surface of the patient. The through-hole 830 is round and has a diameter approximately the same as that of the dielectric element 813. The through-hole 830 is configured to accommodate the corresponding dielectric element 813 after the insulated electrode 800 is assembled.

The supporting members 83 and the dielectric elements 813 are on the same side of the flexible circuit board 811. The supporting members 83 and the heat dissipation reinforcing members 812 are respectively arranged on two opposite sides of the flexible circuit board 811. Each supporting member 83 is arranged in a shape of a sheet and can be made of an insulated material that is soft, chemically stable, light in weight, less easily deformed, and non-toxic and that is formed by integrating polyethylene (PE) material or PET material or thermally conductive silicone sheet or polyurethane, polyethylene, dispersant, a flame retardant, carbon fiber, and the like. Each supporting member 83 is arranged around the corresponding dielectric element 813 to position and support the corresponding adhesive member 84, which can also improve comfort of wearing the insulated electrode 800. The flexible circuit board 811 is sandwiched between the supporting members 83 and the heat dissipation reinforcing members 812. In this embodiment, the supporting members 83 can be flexible foam. The side of the supporting member 83 closer to the body surface of the patient fits the adhesive member 84, and a side of the supporting member 83 away from the body surface of the patient fits the adhesive-backed layer 82 through a biocompatible adhesive arranged on the adhesive-backed layer 82.

The adhesive member 84 is arranged in a shape of a sheet, and has one side attached to side surfaces of the corresponding supporting member 83 and the corresponding dielectric element 813 closer to the body surface of the patient. The other side of the adhesive member 84 fits the corresponding part of the release paper 86 when the insulated electrode 800 is not used and fits the body surface of the patient when the insulated electrode 800 is used, so that the insulated electrode 800 tightly fits the corresponding body surface of the tumor of the patient. The number of adhesive members 84 is the same as the number of the supporting members 83.

The release paper 86 is adhered to another part of the adhesive-backed layer 82 except the electrical functional component 81 through a biocompatible adhesive applied to the adhesive-backed layer 82, and is used for covering the electrical functional component 81, and the supporting members 83 and the adhesive members 84 arranged on the electrical functional component 81, to protect the biocompatible adhesive on the adhesive members 84 and the adhesive-backed layer 82 and avoid pollution of the biocompatible adhesive on the adhesive members 84 and the adhesive-backed layer 82. The release paper 86 is made of an insulated material.

Temperature rise rates on the skin surface of the patient are measured by using a metal plate or metal alloy plate (aluminum metal plate with a thermal conductivity of 237 W/mK) with a thermal conductivity greater than 200 W/mK as the heat dissipation reinforcing member 812 of the insulated electrode 800 and by using an insulated electrode having an epoxy glass cloth laminate that has the same area and thickness as the heat dissipation reinforcing member 812 and that has thermal conductivity of 0.2 W/mK under conditions of the same applied electric field, the same electrode application position, and the same treatment time. The results show that a temperature rise rate on the skin surface of the patient when the insulated electrode having the epoxy glass cloth laminate is used is approximately 0.0223° C./s (temperature test range: 36.5° C. to 39° C.), while a temperature rise rate on the skin surface of the patient when the insulated electrode 800 in the present disclosure is used is approximately 0.0178° C./s (temperature test range: 36.5° C. to 39° C.), which indicates that the temperature rise rate of the insulated electrode using the aluminum metal plate as the heat dissipation reinforcing member 812 is about 20.2% lower than that of the insulated electrode using epoxy glass cloth laminate during actual use.

After the aforementioned test verification, the insulated electrode 800 has the high thermal conductivity due to the use of the heat dissipation reinforcing member 812 that has the thermal conductivity greater than 200 W/mK and uniformly distributed heat dissipation holes 8121, so that the heat accumulated at the epidermis of the patient can quickly dissipated when temperature of the body surface corresponding to the tumor site of the patient rises to a specific threshold after the alternating electric field of a high level is applied for a long time, to achieve a better therapeutic effect of the tumor-treating fields therapy, without a need to reduce the electric field intensity by rapidly reducing the alternating voltage applied to the insulated electrode to reduce the temperature of the body surface corresponding to the tumor site of the patient, which otherwise reduces the therapeutic effect of the tumor-treating fields therapy.

### A Variant Embodiment of the Insulated Electrode Described in the Eighth Embodiment

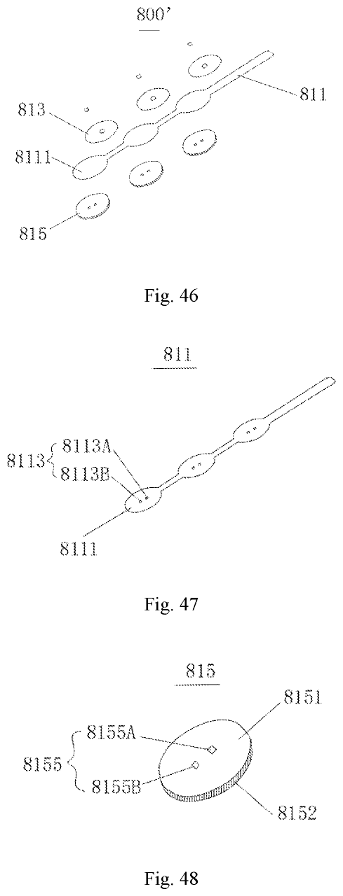

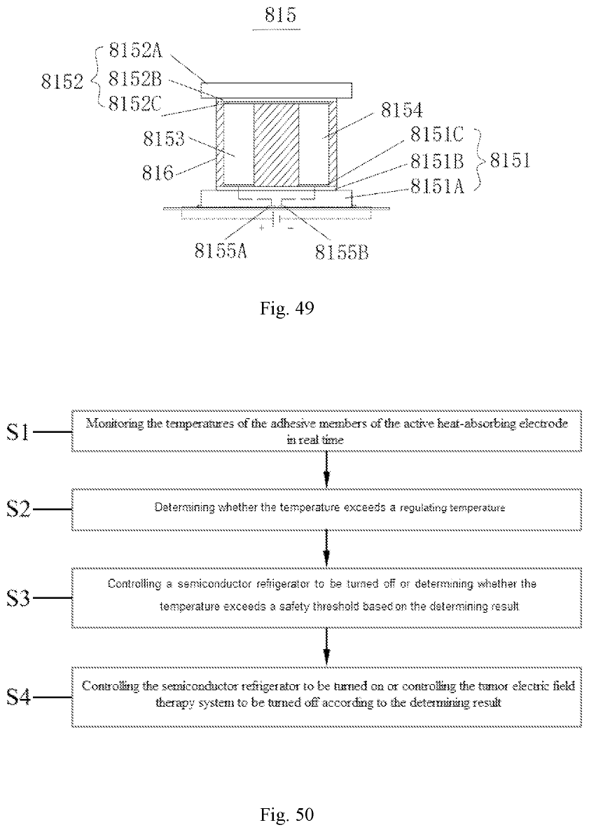

Referring to FIG. 46 to FIG. 49, a specific structure of the insulated electrode 800′ of an active heat-absorbing type is basically the same as that of the insulated electrode 800 in the eighth embodiment, except that the heat dissipation reinforcing member 812 is replaced with a semiconductor refrigerator 815, which can further improve the heat dissipation performance. Only the semiconductor refrigerator 815 is described below. The label numbers of the insulated electrode 800 are still used for other structures of the insulated electrode 800′. For details, refer to images of the insulated electrode 800. Details are not described herein again.

There is a welding portion 8113 exposed from a surface of the main body 8111 of the flexible circuit board 811 that is away from the body surface of the patient, and the welding portion 8113 includes a first welding portion 8113A and a second welding portion 8113B arranged at intervals. The semiconductor refrigerator 815 is welded to the welding portion 8113 to achieve the electrical connection between the flexible circuit board 811 and the semiconductor refrigerator 815. The semiconductor refrigerator 815 can quickly dissipate heat accumulated at the epidermis of the patient, to avoid a low-temperature scald of the body surface corresponding to the tumor site of the patient that is caused because heat is accumulated at the epidermis corresponding to the tumor site of the patient after the alternating voltage is continuously applied to the flexible circuit board 811 and the dielectric elements 813 that are electrically connected for a long time.

The semiconductor refrigerator 815 is arranged in a shape of a circular sheet, and is welded to the welding portion 8113 of the main body 8111 of the flexible circuit board 811, to achieve the electrical connection with the flexible circuit board 811. The semiconductor refrigerator 815 is sandwiched between the main body 8111 of the flexible circuit board 811 and the adhesive-backed layer 82, which can quickly dissipate the heat at the epidermis of the patient where the insulated electrode 800′ is attached. The semiconductor refrigerator 815 has one side arranged on the main body 8111 of the flexible circuit board 811 via soldering and the other side adhered to the adhesive-backed layer 82 through a biocompatible adhesive arranged on the adhesive-backed layer 82. The semiconductor refrigerator 815 has a refrigeration end 8151 closer to the main body 8111 of the flexible circuit board 811, a heat dissipation end 8152 away from the main body 8111 of the flexible circuit board 811, and an N-type semiconductor 8153 and a P-type semiconductor 8154 sandwiched between the refrigeration end 8151 and the heat dissipation end 8152. The semiconductor refrigerator 815 is adhered to the adhesive-backed layer 82 via the heat dissipation end 8152. The N-type semiconductor 8153 and the P-type semiconductor 8154 are mainly prepared via addition of impure bismuth telluride and special treatment. The semiconductor refrigerator 815 is electrically connected to the refrigeration end 8151 and the heat dissipation end 8152 via the N-type semiconductor 8153 and the P-type semiconductor 8154.

The refrigeration end 8151 has welding pads 8155 corresponding to the welding portions 8113 located on the main body 8111 of the flexible circuit board 811. The welding pads 8155 include a positive welding pad 8155A welded to the first welding portion 8113A of the main body 8111 of the flexible circuit board 811 and a negative welding pad 8155B welded to the second welding portion 8113B of the main body 8111 of the flexible circuit board 811. The refrigeration end 8151 is arranged on the flexible circuit board 811 via the welding pad 8155, and electrically connected to the flexible circuit board 811 via the welding pad 8155. The refrigeration end 8151 includes a cold-end ceramic plate 8151A welded to the main body 8111 of the flexible circuit board 811, a cold-end thermal conduction member 8151B arranged on the side of the cold-end ceramic plate 8151A away from the flexible circuit board 811, and two cold-end metal conductors 8151C arranged on the cold-end thermal conduction member 8151B. The two cold-end metal conductors 8151C are arranged in parallel and at intervals and are respectively connected to the N-type semiconductor 8153 and the P-type semiconductor 8154.

The welding pads 8155 are provided on a side of the cold-end ceramic plate 8151A facing toward the flexible circuit board 811. The cold-end ceramic plate 8151A is arranged substantially in a shape of a circular sheet, and has a slightly smaller size than the main body 8111 of the flexible circuit board 811. After the cold-end ceramic plate 8151A and the main body 8111 of the flexible circuit board 811 are welded via the welding portions 8113 and the welding pads 8155, a gap (not shown in the figure) exists therebetween. The gap (not shown in the figure) can be filled in with the sealant 816, which can avoid a short-circuit between the cold-end ceramic plate 8151A and the flexible circuit board 811 caused because water vapor on the body surface of the patient enters the gap (not shown in the figure) of the cold-end ceramic plate 8151A and the main body 8111 of the flexible circuit board 811 and erodes a welded part. The cold-end ceramic plate 8151A is sandwiched between the main body 8111 of the flexible circuit board 811 and the cold-end thermal conduction member 8151B.

The cold-end thermal conduction member 8151B has an integrated circular sheet-shaped structure and is used to fix the cold-end metal conductor 8151C onto the cold-end ceramic plate 8151A. The cold-end thermal conduction member 8151B has a slightly smaller size than the cold-end ceramic plate 8151A. The cold-end thermal conduction member 8151B and the main body 8111 of the flexible circuit board 811 are respectively arranged on two opposite sides of the cold-end ceramic plate 8151A. The cold-end thermal conduction member 8151B is made of a material conducting heat other than electricity. The cold-end thermal conduction member 8151B may be thermal conductive silica. Two recessed spaces (not numbered) respectively accommodating the cold-end metal conductors 8151C are formed by downward recessing top ends of a side of the cold-end thermal conduction member 8151B away from the cold-end ceramic plate 8151A. The two recessed spaces (not numbered) are arranged at intervals and are approximately round.

The two cold-end metal conductors 8151C are respectively arranged in corresponding recessed spaces (not numbered), and protrude from the top end of the cold-end thermal conduction member 8151B. Parts of the two cold-end metal conductors 8151C that protrude from the cold-end thermal conduction member 8151B have the same height. That is, sides of the two cold-end metal conductors 8151C that protrude from the cold-end thermal conduction member 8151B are located at the same plane. The two spaced cold-end metal conductors 8151C have completely the same size. The cold-end metal conductor 8151C is arranged substantially in a shape of a circular sheet, and is made of the same material as the N-type semiconductor 8153 and the P-type semiconductor 8154. The cold-end metal conductor 8151C is preferably made of copper. A diameter of the cold-end metal conductor 8151C is approximately the same as a diameter of a recessed space (not numbered) of the cold-end thermal conduction member 8151B. The two spaced cold-end metal conductors 8151C are arranged on a side of the cold-end thermal conduction member 8151B away from the flexible circuit board 811. The cold-end thermal conduction member 8151B is sandwiched between the cold-end metal conductor 8151C and the cold-end ceramic plate 8151A. Two corresponding conductive traces (not shown in the figure) are provided in each of the cold-end ceramic plate 8151A and the cold-end thermal conduction member 8151B. Ends of two conductive traces on the cold-end ceramic plate 8151A are respectively connected to the welding pads 8155A, 8155B. Two spaced cold-end metal conductors 8151C are arranged on the cold-end ceramic plate 8151A as a group via the cold-end thermal conduction member 8151B.

The N-type semiconductor 8153 and the P-type semiconductor 8154 are both approximately column-shaped. The N-type semiconductor 8153 and the P-type semiconductor 8154 are respectively arranged on the corresponding cold-end metal conductors 8151C as a group. A diameter of the N-type semiconductor 8153 is the same as a diameter of the corresponding cold-end metal conductor 8151C. A diameter of the P-type semiconductor 8154 is the same as a diameter of the corresponding cold-end metal conductor 8151C. The N-type semiconductor 8153 and the P-type semiconductor 8154 are both made of copper. The N-type semiconductor 8153 and the P-type semiconductor 8154 have the same thickness.

The heat dissipation end 8152 includes a hot-end ceramic plate 8152A arranged on the adhesive-backed layer via the biocompatible adhesive arranged on the adhesive-backed layer 82, a hot-end thermal conduction member 8152B arranged on a side of the hot-end ceramic plate 8152A away from the adhesive-backed layer 82 and a hot-end metal conductor 8152C arranged on another side of the hot-end thermal conduction member 8152B. The hot-end metal conductor 8152C is arranged on the N-type semiconductor 8153 and the P-type semiconductor 8154. The hot-end metal conductor 8152C is supported by the N-type semiconductor 8153 and the P-type semiconductor 8154. The N-type semiconductor 8153 is sandwiched between the hot-end metal conductor 8152C and the cold-end metal conductor 8151C. The P-type semiconductor 8154 is sandwiched between the hot-end metal conductor 8152C and another cold-end metal conductor 8151C. That is, the N-type semiconductor 8153 has one end abutting against the corresponding cold-end metal conductor 8151C, and has the other end abutting against a corresponding part of the hot-end metal conductor 8152C. The P-type semiconductor 8154 has one end abutting against another cold-end metal conductor 8151C, and has the other end abutting against a corresponding part of the hot-end metal conductor 8152C. The N-type semiconductor 8153 and the P-type semiconductor 8154 are connected via the hot-end metal conductor 8152C.

The hot-end ceramic plate 8152A is arranged in a shape of a circular sheet, and has approximately the same size as the cold-end ceramic plate 8151A. The hot-end ceramic plate 8152A is sandwiched between the adhesive-backed layer 82 and the hot-end thermal conduction member 8152B. The hot-end thermal conduction member 8152B is sandwiched between the hot-end ceramic plate 8152A and the hot-end metal conductor 8152C. The hot-end thermal conduction member 8152B is arranged in a shape of a circular sheet, and has a slightly smaller size than the hot-end ceramic plate 8152A. A size of the hot-end thermal conduction member 8152B is approximately the same as the size of the cold-end thermal conduction member 8151B. The hot-end thermal conduction member 8152B mounts the hot-end metal conductors 8152C onto the hot-end ceramic plate 8152A. An accommodation space (not numbered) is formed by recessing upward from a side of the hot-end thermal conduction member 8152B that is away from the hot-end ceramic plate 8152A, to accommodate the hot-end metal conductor 8152C. The hot-end thermal conduction member 8152B is made of a material conducting heat other than electricity. The hot-end thermal conduction member 8152B may be thermal conductive silica. The hot-end metal conductor 8152C protrudes from a part of the hot-end thermal conduction member 8152B and is connected to one ends of the N-type semiconductor 8153 and the P-type semiconductor 8154. The hot-end metal conductor 8152C and the cold-end metal conductor 8151C are made of the same material. The hot-end metal conductor 8152C and the cold-end metal conductor 8151C are made of copper.

For the semiconductor refrigerator 815, the cold-end metal conductors 8151C, N-type semiconductor 8153, P-type semiconductor 8154 and the hot-end metal conductor 8152C sandwiched between the hot-end thermal conduction member 8152B of the heat dissipation end 8152 and the cold-end thermal conduction member 8151B of the refrigeration end 8151 are also sealed via a sealant 816, to avoid a short-circuit inside the semiconductor refrigerator 815 and between the welding pads 8155 of the semiconductor refrigerator 815 and the welding portions (not shown in the figure) of the flexible circuit board 811 that is caused because water vapor generated when the refrigeration end 8151 and the heat dissipation end 8152 exchange heat enters the semiconductor refrigerator 815 or the position between the semiconductor refrigerator 815 and the flexible circuit board 811. The semiconductor refrigerator 815 achieves the electrical connection between the positive welding pad 8155A and the negative welding pad 8155B of the cold-end ceramic plate 8151A through the conductive traces (not numbered) which are respectively connected to the positive and negative welding pads 8155A, 8155B of the cold-end ceramic plate 8151A respectively connecting corresponding conductive traces (not numbered) of the cold-end thermal conduction member 8151B, corresponding conductive traces (not numbered) of the cold-end thermal conduction member 8151B respectively being in contact with one ends of the two cold-end metal conductors 8151C arranged on the cold-end thermal conduction member 8151B, through the other ends of the two cold-end metal conductors 8151C respectively being in contact with one end of the N-type semiconductor 8153 and one end of the P-type semiconductor 8154, and the other end of the N-type semiconductor 8153 and the other end of the P-type semiconductor 8154 both in contact with the hot-end metal conductor 8152C arranged on the hot-end thermal conduction member 8152B. The semiconductor refrigerator 815 is welded to the welding portion 8113A of the main body 8111 of the flexible circuit board 811 through the positive welding pad 8155A of the cold-end ceramic plate 8151A, and is welded to the welding portion 8113B of the main body 8111 of the flexible circuit board 811 through the negative welding pad 8155B of the cold-end ceramic plate 8151A, so as to be electrically connected to the flexible circuit board 811, thereby the control signal can be received from the flexible circuit board 811.

The semiconductor refrigerator 815 is made by using the Peltier effect of the semiconductor. When a large amount of heat is accumulated on the body surface corresponding to the tumor site of the patient where the insulated electrode 800′ is attached, the tumor electric field therapy system (not shown in the figure) including the insulated electrode 800′ supplies direct current to the semiconductor refrigerator 815 through the flexible circuit board 811. A current on an internal loop of the semiconductor refrigerator 815 flows from the positive welding pad 8155A of the cold-end ceramic plate 8151A though the cold-end thermal conduction member 8151B sequentially to the cold-end metal conductor 8151C electrically connected to the positive welding pad 8155A of the cold-end ceramic plate 8151A, the N-type semiconductor 8153, the hot-end metal conductor 8152C, the P-type semiconductor 8154, the cold-end metal conductor 8151C electrically connected to the P-type semiconductor 8154, a conductive trace of the cold-end thermal conduction member 8151B electrically connected to the negative welding pad 8155B of the cold-end ceramic plate 8151A to the negative welding pad 8155B of the cold-end ceramic plate 8151A.

Electrons of the P-type semiconductor 8154 of the semiconductor refrigerator 815 sequentially pass through the cold-end metal conductor 8151C in contact with the P-type semiconductor 8154, the conductive traces of the cold-end thermal conduction member 8151B and the cold-end ceramic plate 8151A, the cold-end metal conductor 8151C in contact with the N-type semiconductor 8153, the N-type semiconductor 8153, and the hot-end metal conductor 8152C to reach the P-type semiconductor 8154. At the refrigeration end 8151 of the semiconductor refrigerator 815, when the electrons flow from the P-type semiconductor 8154 to the N-type semiconductor 8153 through the cold-end ceramic plate 8151A, if charge moves from a low-energy position to a high-energy position, heat is absorbed from the external environment. At the heat dissipation end 8152 of the semiconductor refrigerator 815, when the electrons flow from the N-type semiconductor 8153 to the P-type semiconductor 8154 through the hot-end metal conductor 8152C, if charge moves from the high-energy position to the low-energy position, heat is dissipated outward. That is, when direct current is supplied to the semiconductor refrigerator 815 through the flexible circuit board 811, temperature of the refrigeration end 8151 of the semiconductor refrigerator 815 decreases to actively absorb heat from the flexible circuit board 811 through the cold-end ceramic plate 8151A, and temperature of the heat dissipation end 8152 rises to dissipate heat by exchanging heat with external air through the hot-end ceramic plate 8152A. The heat absorbed by the cold-end ceramic plate 8151A is transmitted to the outer side of the insulated electrode 800′ through the cold-end thermal conduction member 8151B, the cold-end metal conductor 8151C, the N-type semiconductor 8153 and P-type semiconductor 8154, the hot-end metal conductor 8152C, the hot-end thermal conduction member 8152B, and the hot-end ceramic plate 8152A, to avoid a low-temperature scald of the epidermis of the patient caused because the heat is accumulated on the body surface of the patient where the insulated electrode 800′ is applied after long-duration and continuous tumor-treating fields therapy, without a need to avoid the low-temperature scald of the epidermis of the patient by discontinuing the treatment, so that the patient has relatively long tumor treatment time, thereby achieving a better therapeutic effect.

Temperature rise rates on the skin surface of the patient are measured by using a semiconductor refrigerator 815 as an active heat absorption insulated electrode 800 and by using an insulated electrode with an epoxy glass cloth laminate that has the same size as the semiconductor refrigerator 815 under conditions of the same applied electric field, the same application position, and the same treatment time. The results show that a temperature rise rate on the skin surface of the patient when the insulated electrode with the epoxy glass cloth laminate is used is approximately 0.0223° C./s (temperature test range: 36.5° C. to 39° C.), while a temperature rise rate on the skin surface of the patient when the insulated electrode 800 in the present disclosure is used is approximately 0.0108° C./s (temperature test range: 36.5° C. to 39° C.), which indicates that the temperature rise rate of the electrode using the semiconductor refrigerator 815 is about 51.5% lower than that of the electrode using epoxy glass cloth laminate during actual use.

After the aforementioned test verification, based on the insulated electrode 800′, the semiconductor refrigerator 815 is arranged on a side of the main body 8111 of the flexible circuit board 811 that is away from the body surface of the patient, and after the controller of the tumor treatment system 1000 supplies direct current to the semiconductor refrigerator 815 through the flexible circuit board 811, the refrigeration end 8151 of the semiconductor refrigerator 815 can actively refrigerate, absorb the heat that is generated on the body surface of the patient due to the long-duration and continuous tumor-treating fields therapy and that is transmitted to the flexible circuit board 811 through the adhesive member 84 and the dielectric element 813, and dissipate the heat to the outer side of the insulated electrode 800′ through the heat dissipation end 8152, to quickly dissipate the heat on the body surface of the tumor of the patient to achieve the purpose of refrigeration, so that the patient can have relatively long treatment time and a better therapeutic effect is ensured, without a need to avoid a low-temperature scald of the epidermis of the patient at the position where the insulated electrode is applied by reducing the alternating voltage of the alternating electrical signal applied to the flexible circuit board 811 or reducing the alternating voltage of the alternating electrical signal applied to the tumor site of the patient through the dielectric element 813.

The adhesive-backed layer 82 can also be provided with an opening (not shown in the figure) at the position corresponding to the hot-end ceramic plate 8152A of the semiconductor refrigerator 815, so that the hot-end ceramic plate 8152A of the semiconductor refrigerator 815 is exposed to air, thereby further improving the heat dissipation effect. According to temperature test data of sample sizes of different human body parts corresponding to different indications, the semiconductor refrigerator 815 can be selectively welded to only the electrode element 810 in a region with high temperature, thereby facilitating a reduction in overall weight of the insulated electrode 800′.

The present disclosure also provides a tumor electric field therapy system 1000, including an insulated electrode 800′ and a controller (not shown in the figure) electrically connected to the insulated electrode 800′. The controller (not shown in the figure) detects the temperatures of the adhesive members 84 in contact with the body surface corresponding to the tumor site of the patient through temperature sensors 814 arranged at the flexible circuit board 811 of the insulated electrode 800′, and then determines whether direct current is to be supplied to the semiconductor refrigerator 815 through the flexible circuit board 811.

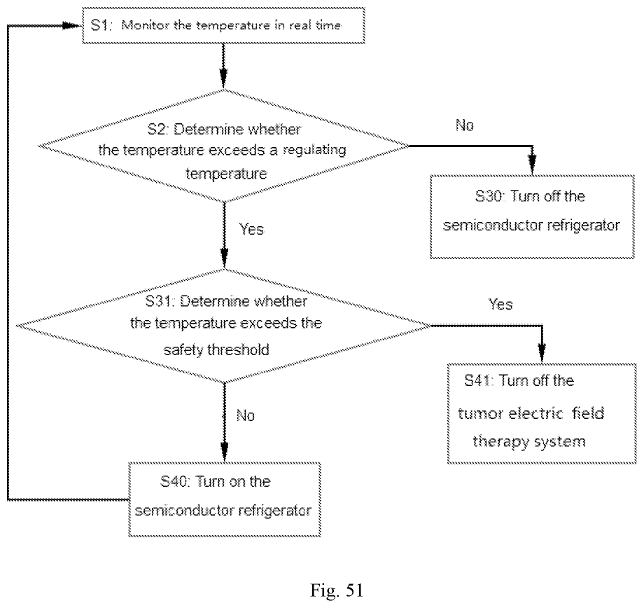

As shown in FIG. 50 and FIG. 51, the present disclosure also provides a temperature control method for a tumor electric field therapy system using the aforementioned insulated electrode 800′, and the tumor electric field therapy system includes the aforementioned insulated electrode 800′ and a controller (not shown in the figure) electrically connected to the insulated electrode 800′. The temperature control method specifically includes the following steps: Step S1: monitoring the temperatures of the adhesive members 84 of the insulated electrode 800′ in real time; Step S2: determining whether the monitored temperature exceeds a regulating temperature; Step S3: controlling the semiconductor refrigerator 815 to be turned off or determining whether the monitored temperature exceeds a safety threshold according to a determining result; Step S4: controlling the semiconductor refrigerator 815 to be turned on or controlling the tumor electric field therapy system to be turned off according to the determining result of whether the temperature exceeds the safety threshold in step S3.

In step S1, each adhesive member 84 of the insulated electrode 800′ has one side adhered to the epidermis of the tumor site of the patient and the other side adhered to the supporting member 83 and the dielectric element 813. In step S1, monitoring the temperature of the adhesive member 84 in real time is specifically implemented through the temperature sensor 814 arranged on the flexible circuit board 811. The temperature of the adhesive member 84 fitting the tumor site of the patient is detected by using the temperature sensor 814, to obtain the skin temperature of the body surface corresponding to the tumor site of the patient fitting the adhesive member 84 by using the temperature sensor 814.

In step S2, determining whether the monitored temperature exceeds the regulating temperature is implemented by comparing the monitored temperature with the regulating temperature.

In step S3, the determining result includes that: the monitored temperature is lower than the regulating temperature or the monitored temperature is higher than the regulating temperature. In step S3, controlling the semiconductor refrigerator 815 to be turned off or determining whether the monitored temperature exceeds a safety threshold according to a determining result specifically includes the following steps: Step S30: when the monitored temperature is lower than the regulating temperature, controlling the semiconductor refrigerator 815 to be turned off; Step S31: when the monitored temperature is higher than or equal to the regulating temperature, determining whether the monitored temperature exceeds the safety threshold. In step S31, determining whether the monitored temperature exceeds the safety threshold is implemented by comparing the monitored temperature with the safety threshold. The difference between the safety threshold and the regulating temperature does not exceed 4° C. to avoid discomfort caused by excessively low temperature. Preferably, the regulating temperature is 390 and the safety threshold is 41°.

In step S4, the determining result of whether the temperature exceeds the safety threshold in step S3 includes that the monitored temperature is higher than or equal to the safety threshold, or the monitored temperature is lower than the safety threshold. In step S4, controlling the semiconductor refrigerator 815 to be turned on or controlling the tumor treatment system to be turned off according to a determining result of whether the temperature exceeds the safety threshold in step S3 specifically includes the following steps: Step S40: when the monitored temperature is lower than the safety threshold, controlling the semiconductor refrigerator 815 to be turned on and performing step S1 to step S3 repeatedly and periodically; Step S41: when the monitored temperature is higher than or equal to the safety threshold, controlling the tumor electric field therapy system to be turned off.

In step S4, controlling the semiconductor refrigerator 815 to be turned on is specifically implemented by controlling the flexible circuit board 811 to supply direct current to the semiconductor refrigerator 815. In step S40, controlling the semiconductor refrigerator 815 to be turned on means that the flexible circuit board 811 is used to supply direct current to the semiconductor refrigerator 815, so that the semiconductor refrigerator 815 enters the turn-on state. In step S41, controlling the tumor electric field therapy system to be turned off means controlling the tumor electric field therapy system to enter a turn-off state, and is specifically implemented by cutting off the power supply of the tumor electric field therapy system.

The step of controlling the semiconductor refrigerator 815 to enter a turn-on state is implemented by supplying direct current to the semiconductor refrigerator 815. The step of controlling the semiconductor refrigerator 815 to enter a turn-off state is specifically implemented by cutting off the direct current supplied to the semiconductor refrigerator 815.

When the semiconductor refrigerator 815 is in ON state, a refrigeration end 8151 of the semiconductor refrigerator 815 can actively absorb the heat that is generated on the body surface corresponding to the tumor site of the patient and that is transmitted to the flexible circuit board 811 through the adhesive member 84 and the dielectric element 813, absorb heat generated by the conductive pad (not shown in the figure) and the dielectric element 813 of the flexible circuit board 811 during operating of the insulated electrode 800′, and quickly dissipate the heat through the heat dissipation end 8152 of the semiconductor refrigerator 815, to quickly reduce temperature of the adhesive member 84 of the insulated electrode 800′, to further reduce temperature of the body surface corresponding to the tumor site of the patient, which can achieve a purpose of reducing the temperature of the body surface corresponding to the tumor site of the patient without needing to reduce the alternating current applied to the insulated electrode 800′ or reduce the alternating electric field applied to the dielectric element 813 through the conductive pad (not shown in the figure) of the flexible circuit board 811, thereby implementing long-duration and continuous tumor-treating fields therapy and improving the therapeutic effect.

In a normal case, after the semiconductor refrigerator 815 is turned on, the temperature of the skin surface slowly decreases, but due to some internal or external factors, there may be a slight probability that the electric field is out of control. To avoid excessively rapid temperature rise caused by the excessively large electric field, when the detected temperature exceeds an upper limit of safe temperature (for example, 41° C.), the power supply of the tumor electric field therapy system is turned off. The tumor electric field therapy system needs to be manually turned on to restart operating.

With such setting, a temperature ladder (39° C., 41° C.) can be controlled to reduce temperature of the skin surface while keeping the tumor electric field therapy system operating to the maximum extent. Compared with the conventional cooling method of directly turning off the tumor electric field therapy system, the temperature control method can provide more time to implement alternating electric field treatment, which improves the therapeutic effect.

It should be noted that relational terms such as “first” and “second” are only used to distinguish one entity or operation from another entity or operation, and do not necessarily claim or imply that there is any such actual relationship or order between these entities or operations. Terms “include”, “include”, or any other variations thereof are intended to cover non-exclusive inclusions, so that processes, methods, articles or devices containing a series of elements not only contain these elements, but also contain other elements which are not clearly listed, or further contain elements which are inherent to these processes, methods, articles or devices. Without being subject to further limitations, an element defined by a phrase “including a . . . ” does not exclude presence of other identical elements in the process, method, article, or device that includes the very element.

The aforementioned describes in detail the method and apparatus provided in the embodiments of the present disclosure. The principle and implementations of the present disclosure are described in this specification through specific examples. The descriptions about the embodiments are merely provided to help understand the methods and core ideas of the present disclosure. In addition, a person of ordinary skill in the art can make variations to the specific implementations and application scopes according to the ideas of the present disclosure. In summary, the content of this specification shall not be construed as any limitation to the present disclosure.

The content disclosed in this patent document contains copyrighted materials. This copyright belongs to the copyright owner. The copyright owner has no objection to any person copying this patent document or patent disclosure when filed in the official records and archives of the Patent and Trademark Office.

1-210. (canceled)

211. A tumor electric field therapy system comprising:
at least two pairs of the insulated electrodes; and
an electric field treatment device electrically connected with the insulated electrodes and including:
an AC signal controller configured to generate control signals with respective time periods and having at least two cyclically switched output states; and
an AC signal generator electrically connected to the AC signal controller and configured to generate an alternating electric signal with an AC voltage and a frequency for tumor-treating fields therapy and to periodically and alternately apply the generated alternating electric signal to the insulated electrodes arranged in pairs to generate alternating electric fields with periodically switching directions between the insulated electrodes arranged in pairs when the AC signal controller is periodically switched between the at least two output states;
wherein the AC signal generator is further configured so that when the alternating electric signal is alternately applied to different pairs of the insulated electrodes, the alternating electric signals applied across the insulated electrodes arranged in pairs each has respective continuous on-time periods for the different pairs of the insulated electrodes, and each of the continuous on-time periods comprises an initial switching-on time period t3, a plurality of intermediate on-time periods and a final switching-off time period t4; and
wherein an AC voltage of the alternating electric signal applied across the insulated electrodes arranged in pair during the intermediate on-time period is set at a specific voltage; and
wherein during the switching-on time period t3, the AC voltage of the alternating electric signal applied across the insulated electrodes arranged in pair rises from 0 to the specific voltage at a constant speed, and wherein the switching-on time period t3 is a set value and a voltage change per millisecond of the AC voltage of the alternating electric signal applied across the insulated electrodes arranged in pair is within 5% of the specific voltage.

212. The tumor electric field therapy system according to claim 211, wherein during the switching-off time period t4, the AC voltage of the alternating electric signal applied across the insulated electrodes arranged in pair decreases from the specific voltage to 0 at a constant speed, and wherein the switching-off time period t4 is a set value and a voltage change per millisecond of the AC voltage of the alternating electric signal applied across the insulated electrodes arranged in pair is within 5% of the specific voltage.

213. The tumor electric field therapy system according to claim 211, wherein the electric field treatment device has preset system parameters which comprise an electric field frequency, an output AC voltage amplitude and a direction switching period of the alternating electrical signal, and wherein the specific voltage is not greater than a peak value of the output AC voltage amplitude of the alternating electrical signal output by the electric field treatment device.

214. The tumor electric field therapy system according to claim 211, wherein during the switching-on time period t3, the voltage change ΔV per millisecond of the AC voltage of the alternating electric signal applied across the insulated electrodes arranged in pairs is obtained as ΔV=V/(t3/t), wherein V is the specific voltage, t is 1 millisecond, and t3 is the switching-on time period of the alternating electric signal and is greater than or equal to 20 milliseconds.

215. The tumor electric field therapy system according to claim 212, wherein during the switching-off time period t4, the voltage change ΔV per millisecond of the AC voltage of the alternating electric signal applied across the insulated electrodes arranged in pairs is obtained as ΔV=V/(t4/t), wherein V is the specific voltage, t is 1 millisecond, and t4 is the switching-off time period of the alternating electric signal and is greater than or equal to 20 milliseconds.

216. The tumor electric field therapy system according to claim 212, wherein the switching-on time period t3 or the switching-off time period t4 of the alternating electric signal is 50 milliseconds.

217. The tumor electric field therapy system according to claim 213, wherein the electric field treatment device comprises an MCU control unit with a reference voltage, a DC power supply control unit in communication with the MCU control unit, an inverter boost control unit in communication with the MCU control unit, a filter control unit connected to the inverter boost control unit, an AC voltage control unit in communication with the filter control unit, a direction control unit in communication with the MCU control unit, a first direction switch electrically connected to the direction control unit and controlling connection and disconnection between the AC voltage control unit and one pair of the insulated electrodes, and a second direction switch electrically connected to the direction control unit and controlling connection and disconnection between the AC voltage control unit and another pair of the insulated electrodes.

218. The tumor electric field therapy system according to claim 217, wherein the MCU control unit comprises a storage module that stores the system parameters of the electric field treatment device, an execution module in communication with the storage module, a digital-to-analog conversion module in communication with the execution module, and a control module that controls the storage module, the execution module, and the digital-to-analog conversion module to perform corresponding operations.

219. The tumor electric field therapy system according to claim 218, wherein the digital-to-analog conversion module has a DAC data register and the DC power supply control unit is in communication with the digital-to-analog conversion module, and wherein the digital-to-analog conversion module is configured to output a corresponding DC electric signal to the DC power supply control unit according to a value stored in DAC data register to start the DC power supply control unit, and wherein the DC power supply control unit outputs a DC electric signal to the inverter boost control unit after being started.

220. The tumor electric field therapy system according to claim 219, wherein the AC voltage of the alternating electric signal applied across the insulated electrodes arranged in pairs rising from 0 to the specific voltage at a constant speed during the switching-on time period t3 is achieved by the MCU control unit controlling the DC power supply unit to output a DC electric signal with a voltage increased at a constant speed to the inverter boost control unit based on the direction switching period of the alternating electric signal obtained by the execution module.

221. The tumor electric field therapy system according to claim 219, wherein the AC voltage of the alternating electric signal applied across the insulated electrodes arranged in pairs decreasing from the specific voltage to 0 at a constant speed during the switching-off time period t4 is achieved by the MCU control unit controlling the DC power supply unit to output a DC electric signal with a voltage decreased at constant speed to the inverter boost control unit based on the direction switching period of the alternating electric signal obtained by the execution module.

222. The tumor electric field therapy system according to claim 219, wherein the MCU control unit is further configured to calculate a voltage output increment or decrement of the digital-to-analog conversion module per millisecond based on the reference voltage of the MCU control unit, the AC voltage change of the AC voltage control unit per millisecond, the specific voltage and the DAC data register value corresponding to the specific voltage; and wherein the voltage output increment or decrement of the digital-to-analog conversion module per millisecond is determined as ΔV_{DAC}=(3.3*1000*ΔV*DAC)/(4096*V), wherein ΔV_{DAC} is the voltage output increment or decrement of the digital-to-analog conversion module per millisecond and is measured in millivolts; 3.3 is the reference voltage of the MCU and is measured in volts; the value corresponding to the reference voltage of the MCU and stored in the DAC data register is 4096 and equal to 2¹²; ΔV is the AC voltage change of the AC voltage control unit per millisecond and is measured in volts; V is the specific voltage and is measured in volts; and DAC is a DAC data register value corresponding to the specific voltage and strode in the DAC data register.

223. The tumor electric field therapy system according to claim 219, wherein the MCU control unit calculates a numerical output increment of the DAC data register of the digital-to-analog conversion module per millisecond based on a DC voltage change of the DC power supply control unit per millisecond.

224. The tumor electric field therapy system according to claim 219, wherein the MCU control unit calculates a numerical output decrement of the DAC data register of the digital-to-analog conversion module per millisecond based on a DC voltage change of the DC power supply control unit per millisecond.

225. The tumor electric field therapy system according to claim 211, wherein the AC signal controller has various output states which have respective and non-overlapped time periods corresponding thereto, and the AC signal controller only has one output state in each time period.

226. The tumor electric field therapy system according to claim 211, wherein the at least two pairs of the insulated electrodes comprise a first pair of the insulated electrodes and a second pair of the insulated electrodes all arranged on a torso surface of a patient; and wherein the AC signal controller is configured to generate a periodic control signal having a first output state with a duration between 500 ms and 980 ms and a second output state with a duration between 500 ms and 980 ms; and wherein the AC signal generator generates a first AC signal applied to the first pair of the insulated electrodes when the control signal is in the first output state, and generates a second AC signal applied to the second pair of the insulated electrodes when the control signal is in the second output state, and wherein a switch between applying the first AC signal to the first pair of the insulated electrode and applying the second AC signal to the second pair of the insulated electrodes is achieved by switching between the first output state and the second output state of the control signal.

227. The tumor electric field therapy system according to claim 226, wherein the first output state has a duration with a first time period T1 and the second output state has a duration with a second time period T2, and wherein the first time period T1 is same as the second time period T2.

228. The tumor electric field therapy system according to claim 227, wherein the first time period T1 and the second time period T2 are both 50% of an operating period.

229. The tumor electric field therapy system according to claim 227, wherein the first AC signal has an increasing amplitude during the switching-on time period t3 and a decreasing amplitude during the switching-off time period t4 within each of the first time periods T1; and
where the second AC signal has an increasing amplitude during the switching-on time period t3 and a decreasing amplitude during the switching-off time period t4 within each of the second time periods T2.

230. The tumor electric field therapy system according to claim 229, wherein the switching-on time period t3 and the switching-off time period t4 each has a duration both shorter than 10% of a duration of the first or second time period T1, T2.
